(19) United States
(12) Patent Application Publication
(10) Pub. No.: US 2019/0388522 A1
(43) Pub. Date: Dec. 26, 2019

(54) METHODS FOR TREATING DISORDERS ASSOCIATED WITH ANGIOGENESIS AND NEOVASCULARIZATION

(71) Applicant: Iconic Therapeutics, Inc., South San Francisco, CA (US)

(72) Inventors: Gabriela BURIAN, South San Francisco, CA (US); William GREENE, South San Francisco, CA (US); Kirk DORNBUSH, South San Francisco, CA (US)

(21) Appl. No.: 16/480,804

(22) PCT Filed: Jan. 25, 2018
(86) PCT No.: PCT/US18/15257

§ 371 Date: Jul. 25, 2019

Related U.S. Application Data
(60) Provisional application No. 62/450,452, filed on Jan. 25, 2017.

(51) Int. Cl. A61K 38/48; A61K 39/395; A61K 9/00; A61P 27/02; C07K 16/22; A61K 38/17

(52) U.S. Cl. CPC A61K 38/4846; A61K 39/3955; A61K 9/0019; A61K 9/0048; A61P 27/02; A61K 2039/505; A61K 38/179; C07K 2317/24; C07K 2317/76; C07K 2317/55; C07K 16/22

(57) ABSTRACT

Provided herein are methods and compositions for the treatment of diseases associated with angiogenesis and neovascularization. In one aspect, the invention relates to a method for treating a condition in an eye of a patient in need thereof comprising administering to the patient in multiple dosing sessions, an effective amount of an immunoconjugate dimer, wherein the monomer subunits of the dimer each comprises a mutated human factor VIIa (fVIIa) protein conjugated to the human immunoglobulin G1 (IgG1) Fc domain and a VEGF inhibitor, wherein the administration results in an improved outcome compared to a patient having been administered the VEGF inhibitor alone.

### CROSS-REFERENCE TO RELATED APPLICATIONS

This application claims the benefit of U.S. Provisional Application Ser. No. 62/450,452, filed on Jan. 25, 2017, which is herein incorporated by reference in its entirety.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is ICTH_006_00US_ST25. The text file is 24 KB, was created on Jan. 25, 2017, and is being submitted electronically via EFS-Web.

### BACKGROUND OF THE INVENTION

Age-related macular degeneration (AMD) refers to the chronic, progressive degenerative pathology of the macula that results in loss of central vision. According to the Macula Vision Research Foundation and the National Eye Institute, as many as fifteen million people in the United States suffer from some form of AMD, with similar numbers in Europe and other continents. Neovascular AMD (also revered to as exudative or “wet” AMD) is the leading cause of severe vision loss and blindness in elderly patients over the age of fifty in the industrialized world. In the United States alone, more than 1.5 million people suffer from wet AMD. It is expected that AMD incidence and prevalence will further increase with the ageing population, thus leading to a significant increase in the number of patients with wet AMD in the United States and worldwide.

Tissue factor (TF) is a cytokine receptor present on vascular endothelial cells. It is an integral membrane glycoprotein with an intracellular terminal domain, a transmembrane domain, and an extracellular binding domain for Factor VII (FVII) and Factor VIIa (FVIIa). TF has been implicated in the process of angiogenesis and the inflammatory cascade of cytokine release, both processes in the pathogenesis of neovascular AMD and certain cancers.

Choroidal neovascularization (CNV) is the process in which new blood vessels grow in the choroid layer of the eye, and is associated with wet AMD. Therapies targeting vascular endothelial growth factor (VEGF) are currently the standard of clinical care for wet AMD. However, due to the multifaceted aspects of choroidal neovascularization and AMD pathogenesis, targeting VEGF alone is most likely insufficient to halt the progression of the disease towards the advanced CNV-associated degenerative processes.

There is an unmet medical need for new therapeutic strategies for choroidal neovascularization and age-related macular degeneration. The present invention addresses this and other needs.

### SUMMARY OF THE INVENTION

In one aspect, the present invention provides a method for preventing, inhibiting, or reversing wet age-related macular degeneration (AMD) in an eye of a patient in need thereof, comprising, administering to the patient in multiple dosing sessions: (a) an effective amount of an immunoconjugate dimer, wherein the monomer subunits of the dimer each comprises a mutated human factor VIIa (fVIIa) protein conjugated to the human immunoglobulin G1 (IgG1) Fc domain; and (b) a VEGF inhibitor: wherein the administration results in an improved best-corrected visual acuity (BCVA) outcome, improved reduction in mean choroidal neovascularization (CNV) lesion area, improved reduction in CNV exudation, or improved durability of treatment compared to patients having been administered the VEGF inhibitor alone.

In one aspect, the present invention provides a method for preventing, inhibiting, or reversing ocular neovascularization in an eye of a patient in need thereof, comprising, administering to the patient in multiple dosing sessions, a composition comprising: (a) an effective amount of an immunoconjugate dimer, wherein the monomer subunits of the dimer each comprises a mutated human factor VIIa (fVIIa) protein conjugated to the human immunoglobulin G1 (IgG1) Fc domain; and (b) a VEGF inhibitor; wherein the administration results in an improved best-corrected visual acuity (BCVA) outcome, improved reduction in mean choroidal neovascularization (CNV) lesion area, improved reduction in CNV exudation, or improved durability of treatment compared to patients having been administered the VEGF inhibitor alone.

In one aspect, the present invention provides a method for reversing tumor neovascularization in an eye of a patient in need thereof, comprising administering to the patient in multiple dosing sessions a composition comprising: (a) an effective amount of an immunoconjugate dimer, wherein the monomer subunits of the dimer each comprises a mutated human factor VIIa (fVIIa) protein conjugated to the human immunoglobulin G1 (IgG1) Fc domain; and (b) a VEGF inhibitor, wherein the administration results in an improved best-corrected visual acuity (BCVA) outcome, improved reduction in mean choroidal neovascularization (CNV) lesion area, improved reduction in CNV exudation, or improved durability of treatment compared to patients having been administered the VEGF inhibitor alone.

In some embodiments, the VEGF inhibitor comprises an anti-VEGF antibody. In some embodiments, the immunoconjugate dimer is a homodimer. In some embodiments, the immunoconjugate dimer is a heterodimer.

In one aspect, the present invention provides a method for preventing, inhibiting, or reversing wet age-related macular degeneration (AMD) in an eye of a patient in need thereof, comprising, administering to the patient in multiple dosing sessions: (a) an effective amount of an immunoconjugate dimer, wherein the monomer subunits of the dimer each comprises a mutated human factor VIIa (fVIIa) protein conjugated to the human immunoglobulin G1 (IgG1) Fc domain; and (b) a VEGF inhibitor, wherein the administration results in an improved best-corrected visual acuity (BCVA) outcome, improved reduction in mean choroidal neovascularization (CNV) lesion area, improved reduction in CNV exudation, or improved durability of treatment compared to patients having been administered the VEGF inhibitor alone.

In one aspect, the present invention provides a method for preventing, inhibiting, or reversing ocular neovascularization in an eye of a patient in need thereof, comprising, administering to the patient in multiple dosing sessions: (a) an effective amount of an immunoconjugate dimer, wherein the monomer subunits of the dimer each comprises a mutated human factor VIIa (fVIIa) protein conjugated to the human immunoglobulin G1 (IgG1) Fc domain; and (b) a VEGF inhibitor; wherein the administration results in an improved best-corrected visual acuity (BCVA) outcome, improved reduction in mean choroidal neovascularization (CNV) lesion area, improved reduction in CNV exudation, or improved durability of treatment compared to patients having been administered the VEGF inhibitor alone.

In one aspect, the present invention provides a method for preventing, inhibiting, or reversing tumor neovascularization in an eye of a patient in need thereof, comprising, administering to the patient in multiple dosing sessions: (a) an effective amount of an immunoconjugate dimer, wherein the monomer subunits of the dimer each comprises a mutated human factor VIIa (fVIIa) protein conjugated to the human immunoglobulin G1 (IgG1) Fc domain; and (b) a VEGF inhibitor, wherein the administration results in an improved best-corrected visual acuity (BCVA) outcome, improved reduction in mean choroidal neovascularization (CNV) lesion area, improved reduction in CNV exudation, or improved durability of treatment compared to patients having been administered the VEGF inhibitor alone.

In some embodiments, the mutated factor VII protein exhibits a decreased coagulation response in a mammalian host, as compared to a wild-type factor VII protein.

In some embodiments, at least one of the monomer subunits of the immunoconjugate comprises a mutated human fVIIa domain comprising a single point mutation at Lys341 or Ser 344. In a further embodiment, the single point mutation is to an alanine. In a further embodiment, the single point mutation is Lys341 to Ala341. In a further embodiment, the single point mutation is Ser344 to Ala344.

In some embodiments, the ocular neovascularization is secondary to proliferative diabetic retinopathy, wet age-related macular degeneration (AMD), retinopathy of prematurity (ROP), or neovascular glaucoma. In some embodiments, the ocular neovascularization is choroidal neovascularization. In some embodiments, the choroidal neovascularization is secondary to wet AMD.

In some embodiments, the patient has been previously diagnosed with wet AMD in the eye. In a further embodiment, the eye of the patient has not been previously treated for choroidal neovascularization or wet AMD. In some embodiments, the patient has previously been treated for choroidal vascularization with anti-vascular endothelial growth factor (VEGF) therapy, laser therapy, or surgery.

In some embodiments, the administering comprises intravitreal injection at each dosing session. In some embodiments, the administering comprises suprachoroidal injection at each dosing session.

In some embodiments, multiple dosing sessions comprise two or more, three or more, four or more, five or more, six or more, seven or more, eight or more, nine or more, ten or more, eleven or more, twelve or more, thirteen or more, fourteen or more, fifteen or more, sixteen or more, seventeen or more, eighteen or more, nineteen or more, or twenty or more dosing sessions. In some embodiments, wherein the multiple dosing sessions comprise 12 to 24 dosing sessions. In some embodiments, the administering comprises intravitreal injection into the eye of the patient once every 28 days, once every 30 days, or once every 35 days.

In some embodiments, the immunoconjugate comprises intravitreal the amino acid sequence of SEQ ID NO: 2 or 3. In a further embodiment, the immunoconjugate comprises the amino acid sequence of SEQ ID NO:2. In a further embodiment, the immunoconjugate comprises the amino acid sequence of SEQ ID NO:3. In some embodiments, the immunoconjugate is encoded by a polynucleotide sequence comprising SEQ ID NO:4. In some embodiments, the immunoconjugate is encoded by a polynucleotide sequence comprising SEQ ID NO:5. In some embodiments, the administering comprises intravenous or intratumoral administration.

In some embodiments, the improved reduction in CNV lesion area or CNV exudation is reduced by at least about 5%, at least about 10%, at least about 20%, at least about 30%, at least about 40%, or at least about 50%. In some embodiments, the improved durability of treatment is increased by at least about 5%, at least about 10%, at least about 20%, at least about 30%, at least about 40%, or at least about 50%.

In some embodiments, subsequent to the multiple dosing sessions, the retinal thickness of the eye of the patient is reduced in the eye of the patient, as compared to the retinal thickness of the eye of patients having been administered the VEGF inhibitor alone. In some embodiments, the retinal thickness is reduced by at least about 50 μm, at least about 100 μm, at least about 150 μm, at least about 175 μm, at least about 200 μm, at least about 225 μm, or at least about 250 μm. In some embodiments, the retinal thickness is reduced by at least about 5%, at least about 10%, at least about 20%, at least about 30%, at least about 40%, or at least about 50%. In a further embodiment, the decreased retinal thickness is decreased central retinal subfield thickness (CST), decreased center point thickness (CPT), or decreased central foveal thickness (CFT).

In some embodiments, the intraocular pressure (IOP) in the eye of the patient is measured prior to each dosing session. In some embodiments, the IOP in the eye of the patient about 20 minutes, about 30 minutes, about 40 minutes, about 50 minutes, or about 1 hour after each dosing session. In some embodiments, the IOP in the eye of the patient about 20 minutes, about 30 minutes, about 40 minutes, about 50 minutes, or about 1 hour prior to each dosing session. In further embodiments, the IOP is measured via tonometry.

In some embodiments, the VEGF inhibitor is present in the same composition as the immunoconjugate. In some embodiments, the VEGF inhibitor is present in a different composition than the immunoconjugate. In some embodiments, the anti-VEGF antibody is ranibizumab. In some embodiments, the dosage of ranibizumab is from about 0.2 mg to about 1 mg. In a further embodiment, the dosage of ranibizumab is 0.3 mg, 0.4 mg, 0.5 mg, 0.6 mg, or 0.7 mg. In some embodiments, the ranibizumab is administered to the eye of the patient via an intravitreal injection.

In some embodiments, the VEGF inhibitor is administered at a dosage from about 0.2 mg to about 0.7 mg. In some embodiments, the VEGF inhibitor is administered at a dosage of about 0.6 mg. In some embodiments, wherein the VEGF inhibitor is administered at a dosage of 0.6 mg. In some embodiments, the VEGF inhibitor is administered at a dosage of about 0.3 mg. In some embodiments, the VEGF inhibitor is administered at a dosage of 0.3 mg.

In some embodiments, the multiple dosing sessions comprise administration once per month. In a further embodiment, the multiple dosing sessions comprise administration once per month for the first three months, followed by monthly treatments in months 4-12 only when CNV activity is observed.

In some embodiments, wherein the composition comprising the effective amount of the VEGF inhibitor is administered to the eye of the patient via an intravitreal injection. In some embodiments, the composition comprising the effective amount of the VEGF inhibitor is administered at each of the multiple dosing sessions. In some embodiments, each dosing session comprises the administration of between about 200 μg and about 600 μg of the immunoconjugate dimer. In some embodiments, the administration is about 300 μg of the immunoconjugate dimer. In some embodiments, the administration is about 600 μg of the immunoconjugate dimer.

In some embodiments, the outcome is measured at least 6 months after beginning treatment. In some embodiments, the patient is a human.

In another aspect, there is provided a composition for use in a method of treatment, wherein the composition comprises an immunoconjugate dimer, wherein at least one of the monomer subunits of the dimer comprises a mutated human factor VIIa (fVIIa) protein conjugated to the human immunoglobulin G1 (IgG1) Fc domain; and wherein the method of treatment further comprises administration of a VEGF inhibitor; and wherein the method of treatment comprises administration of multiple doses of the immunoconjugate and VEGF inhibitor. In an embodiment, the composition may be for use in treating neovascularization and/or angiogenesis. In an embodiment, the composition may be for use in treating ocular disorders. In an embodiment, the composition may be for use in treating ocular disorders associated with neovascularization and/or angiogenesis. In an embodiment, the composition may be for use in treating wet AMD, ocular neovascularization, or tumor neovascularization. In an embodiment, the monomer subunits of the immunoconjugate dimer each comprises a mutated human factor VIIa (fVIIa) protein conjugated to the human immunoglobulin G1 (IgG1) Fc domain. In another embodiment, the immunoconjugate comprises two dimerized immunoglobulin (Ig) Fc monomers and a mutated factor VII protein, wherein the mutated factor VII protein is fused to only one of the Fc monomers. The compositions may be for use in treating an eye of a patient in need thereof, and an effective amount of the composition may be administered to the patient in multiple dosing sessions to result in an improved best-corrected visual acuity (BCVA) outcome, improved reduction in mean choroidal neovascularization (CNV) lesion area, improved reduction in CNV exudation, or improved durability of treatment compared to patients having been administered the VEGF inhibitor alone.

In another aspect, there is provided a composition for use in treating, preventing, inhibiting, or reversing wet age-related macular degeneration (AMD), wherein the composition comprises (a) an immunoconjugate dimer, wherein at least one of the monomer subunits of the dimer comprises a mutated human factor VIIa (fVIIa) protein conjugated to the human immunoglobulin G1 (IgG1) Fc domain; and optionally (b) a VEGF inhibitor. For compositions comprising only (a), a separate composition comprising a VEGF inhibitor may also be administered. In an embodiment, the monomer subunits of the immunoconjugate dimer each comprises a mutated human factor VIIa (fVIIa) protein conjugated to the human immunoglobulin G1 (IgG1) Fc domain. In another embodiment, the immunoconjugate comprises two dimerized immunoglobulin (Ig) Fc monomers and a mutated factor VII protein, wherein the mutated factor VII protein is fused to only one of the Fc monomers. The compositions may be for use in treating an eye of a patient in need thereof, and an effective amount of the composition may be administered to the patient in multiple dosing sessions to result in an improved best-corrected visual acuity (BCVA) outcome, improved reduction in mean choroidal neovascularization (CNV) lesion area, improved reduction in CNV exudation, or improved durability of treatment compared to patients having been administered the VEGF inhibitor alone.

In another aspect, there is provided a composition for use in treating, preventing, inhibiting, or reversing ocular neovascularization, wherein the composition comprises (a) an immunoconjugate dimer, wherein at least one of the monomer subunits of the dimer comprises a mutated human factor VIIa (fVIIa) protein conjugated to the human immunoglobulin G1 (IgG1) Fc domain; and optionally (b) a VEGF inhibitor. For compositions comprising only (a), a separate composition comprising a VEGF inhibitor may also be administered. In an embodiment, the monomer subunits of the immunoconjugate dimer each comprises a mutated human factor VIIa (fVIIa) protein conjugated to the human immunoglobulin G1 (IgG1) Fc domain. In another embodiment, the immunoconjugate comprises two dimerized immunoglobulin (Ig) Fc monomers and a mutated factor VII protein, wherein the mutated factor VII protein is fused to only one of the Fc monomers. The compositions may be for use in treating an eye of a patient in need thereof, and an effective amount of the composition may be administered to the patient in multiple dosing sessions to result in an improved best-corrected visual acuity (BCVA) outcome, improved reduction in mean choroidal neovascularization (CNV) lesion area, improved reduction in CNV exudation, or improved durability of treatment compared to patients having been administered the VEGF inhibitor alone.

In another aspect, there is provided a composition for use in treating or reversing tumor neovascularization, wherein the composition comprises (a) an immunoconjugate dimer, wherein at least one of the monomer subunits of the dimer comprises a mutated human factor VIIa (fVIIa) protein conjugated to the human immunoglobulin G1 (IgG1) Fc domain; and optionally (b) a VEGF inhibitor. For compositions comprising only (a), a separate composition comprising a VEGF inhibitor may also be administered. In an embodiment, the monomer subunits of the immunoconjugate dimer each comprises a mutated human factor VIIa (fVIIa) protein conjugated to the human immunoglobulin G1 (IgG1) Fc domain. In another embodiment, the immunoconjugate comprises two dimerized immunoglobulin (Ig) Fc monomers and a mutated factor VII protein, wherein the mutated factor VII protein is fused to only one of the Fc monomers. The compositions may be for use in treating an eye of a patient in need thereof, and an effective amount of the composition may be administered to the patient in multiple dosing sessions to result in an improved best-corrected visual acuity (BCVA) outcome, improved reduction in mean choroidal neovascularization (CNV) lesion area, improved reduction in CNV exudation, or improved durability of treatment compared to patients having been administered the VEGF inhibitor alone.

In another aspect, there is provided a composition for use in a method of treatment, wherein the composition comprises an 0.3 mg, 0.6 mg, or 0.9 mg of an immunoconjugate dimer, wherein monomer subunits of the dimer each comprises a mutated human factor VIIa (fVIIa) protein conjugated or fused to the human immunoglobulin G1 (IgG1) Fc domain; and wherein the method of treatment comprises administration of 0.5 mg of ranibizumab or 2.0 mg of aflibercept; and wherein multiple doses of the ranibizumab or aflibercept, an multiple does of the immunoconjugate dimer are administered monthly for at least three months; and wherein the composition is for use in treating ocular disorders associated with neovascularization and/or angiogenesis; and wherein the composition is for use in treating an eye of a patient in need thereof to result in an improved best-corrected visual acuity (BCVA) outcome, improved reduction in mean choroidal neovascularization (CNV) lesion area, improved reduction in CNV exudation, improved durability of treatment, or no exudative presence of subretinal fluid, intraretinal fluid, and/or rubretinal pigment epithelium fluid compared to patients having been administered the VEGF inhibitor alone.

### BRIEF DESCRIPTION OF THE FIGURES

FIG. 1 is a non-limiting diagram of two ICON-1 immunoconjugate embodiments and one ICON-1.5 immunoconjugate embodiment of the present invention.

FIG. 2 is a graph of the rate of the intrinsic factor Xase complex (Fxase) hydrolysis (increase in absorbance at 405 nm-mOD/min) as a function of time.

FIG. 3 is a graph of thrombin generation by the known inhibitor of coagulation, active site inhibited FVIIa (FVIIai), as a function of time in normal pooled plasma.

FIG. 4 is a graph of thrombin generation by hI-con1, as a function of time in normal pooled plasma.

FIG. 5 is a graph of thrombin generation by human Factor VIIa and hI-con1 as a function of time in FVII-depleted plasma.

FIG. 6 is a graph of thrombin generation by hI-con1, as a function of time in rabbit plasma.

FIG. 7 is a graph of thrombin generation by hI-con1 or FVIIai as a function of time in centrifuged rabbit plasma.

FIG. 8 is a graph showing the percent CNV in the pig as a function of intravitreal dose of hI-con1. Intravitreal injections (100 μL/eye) of solutions of hI-con1 (0.25, 0.5, 1.0 and 2.0 mg/mL) were injected into both eyes of mini-pigs on Day 10, control animals received 100 μL of formulation buffer. On Day 14 the animals were sacrificed and the % CNV was determined.

FIG. 9 is a graph showing the percent CNV in the pig as a function of intravitreal dose of a 100 kDa fragment of hI-con1. Intravitreal injections (100 μL/eye) of solutions of hI-con1 (0.25, 0.5, 1.0 and 2.0 mg/mL) were injected into both eyes of mini-pigs on Day 10; control animals received 100 μL of formulation buffer. On Day 14 the animals were sacrificed and the % CNV was determined.

FIG. 10 is an image representing the distribution of patients within treatment arms with indications for dosing schedules that are further delineated by treatment induction and treatment extension.

FIG. 11 is a tabulation of the baseline CNV lesion area (mm²) for the patients within each of the treatment arms compared with the mean CNV lesion area change from baseline.

FIG. 12 is a tabulation of the change in lesion size for the patients within each of the treatment arms, the total number of patients exhibiting the change (or no change), and the corresponding change in BCVA.

FIG. 13 is a graph depicting the proportional change/no change in lesion size for the patients within each of the treatment arms.

FIG. 14 is a graph displaying the mean change in CST over time in the study eyes in all treatment arms.

FIG. 15 is a graph displaying the mean change in BCVA score over time in the study eyes in all treatment arms.

FIG. 16 is a tabulation of the gain of BCVA from baseline at month 6 for patients in all treatment arms. The figure further identifies the proportion of patients with BCVA with ≥71 at month 6, proportion of patients with BCVA with ≤33 letters at month 6, and the mean BCVA change in letter score at month 6.

FIG. 17 is a tabulation of the fluid type (IRF, SRF, and Sub-RPE) at baseline for patients within each of the treatment arms. The figure further identifies the number of patients with no IRF, no SRF, no sub-RPE, and dry retina at 6 months for each of the treatment arms.

FIG. 18 is a graph identifying the percent of patients exhibiting dry retina within each of the treatment arms.

FIG. 19 is a tabulation of the patients within each of the treatment arms that received study drug retreatment, and a further indication of the number of retreatments.

FIG. 20 is a graph identifying the time from treatment end to first retreatment for patients in each of the treatment arms.

FIG. 21 is a graph identifying the durability of treatment for patients within each of the treatment arms. Durability of treatment is represented as a proportion of the patients in each of the treatment arms that did not require retreatment

FIG. 22 is a graph depicting a dose-dependent reduction in lesion fluorescence in pigs that underwent bilateral laser induction of laser spots in the eyes; measured at day 14 post-induction. The pigs were administered a vehicle control, ICON-1 at 300 μg, ICON-1 at 600 μg, ICON-1 at 900 μg, or Eylea at 2 mg.

FIG. 23 is a graph depicting a reduction in lesion fluorescence in pigs that underwent bilateral laser induction laser spots in the eyes, measured at day 14 post-induction. The pigs were administered a vehicle control, ICON-1 at 600 μg, Eylea at 2 mg, or ICON-1 at 600 μg and Eylea at 2 mg.

### DETAILED DESCRIPTION OF THE INVENTION

The term “a” or “an” may refer to one or more of that entity, i.e. can refer to plural referents. As such, the terms “a” or “an”, “one or more” and “at least one” are used interchangeably herein. In addition, reference to “an element” by the indefinite article “a” or “an” does not exclude the possibility that more than one of the elements is present, unless the context clearly requires that there is one and only one of the elements.

Reference throughout this specification to “one embodiment”, “an embodiment”, “one aspect”, or “an aspect” means that a particular feature, structure or characteristic described in connection with the embodiment is included in at least one embodiment of the present disclosure. Thus, the appearances of the phrases “in one embodiment” or “in an embodiment” in various places throughout this specification are not necessarily all referring to the same embodiment. Furthermore, the particular features, structures, or characteristics can be combined in any suitable manner in one or more embodiments.

As used herein, in particular embodiments, the terms “about” or “approximately” when preceding a numerical value indicates the value plus or minus a range of 10%.

As used herein, a “patient” refers to a warm-blooded animal, e.g., rat, mouse, sheep, cow, pig, guinea pig, non-human primate, human primate, wherein males and/or females may be a patient.

As used herein, “classic CNV” means a well-defined CNV area that results in vision that is between 20/25 and 20/400, but may be worse than 20/800.

As used herein, “occult CNV” means a poorly delineated CNV area that exhibits less leakage than classic CNV, and results in vision that is between 20/25 and 20/400, but may be worse than 20/800.

As used herein, “improved” means at least a 5% improvement of the outcome for which “improved” modifies, as compared to a corresponding control, e.g., an improved reduction in mean CNV lesion area is at least a 5% improvement of the mean CNV lesion area as compared to the mean CNV lesion area under control conditions.

As used herein, “antibody” includes any monoclonal antibody, polyclonal antibody, multispecific antibody, bispecific antibody, single-chain antibody, a single-chain variable fragment (scFv) of an antibody, FAB fragment of an antibody, and fragments thereof.

As used herein, “CNV activity” includes new or increased fluid and/or leakage, hemorrhage and/or lesion, persistent fluid, and decreased chorioretinal blood flow at the site of the perceived CNV lesion.

### Angiogenesis

Pathologic angiogenesis, the induction of the growth of existing blood vessels from the vessels in surrounding tissue, is observed in a variety of diseases, typically triggered by the release of specific growth factors for vascular endothelial cells. Pathologic angiogenesis can result in neovascularization, i.e., the creation of new blood vessels, enabling solid tumor growth and metastasis, causing visual malfunction in ocular disorders, promoting leukocyte extravasation in inflammatory disorders, and/or influencing the outcome of cardiovascular diseases such as atherosclerosis.

In one aspect of the present invention, methods for treating a patient having a disease associated with neovascularization and/or angiogenesis, such as cancer, rheumatoid arthritis, the exudative (“wet”) form of macular degeneration, and/or atherosclerosis are provided. As described herein, administration may be local or systemic, depending upon the type of pathological condition involved in the therapy. As used herein, the term “patient” includes both humans and other species, including other mammal species. The invention thus has both medical and veterinary applications. In veterinary compositions and treatments, immunoconjugates are constructed using targeting and effector domains derived from the corresponding species.

In the aspects provided herein, methods for treating a patient for a disease associated with angiogenesis and/or neovascularization are provided. In one embodiment, the disease associated with neovascularization and/or angiogenesis is wet AMD. In other embodiments, the disease associated with neovascularization and/or angiogenesis is a cancer (ocular melanoma), atherosclerosis, rheumatoid arthritis, oculr melanoma, diabetic macular edema (DME), macular edema following retinal vein occlusion (RVO), proliferative diabetic retinopathy, wet age-related macular degeneration (AMD), reinopathy of prematurity (ROP), or neovascular glaucoma.

### Immunoconjugates

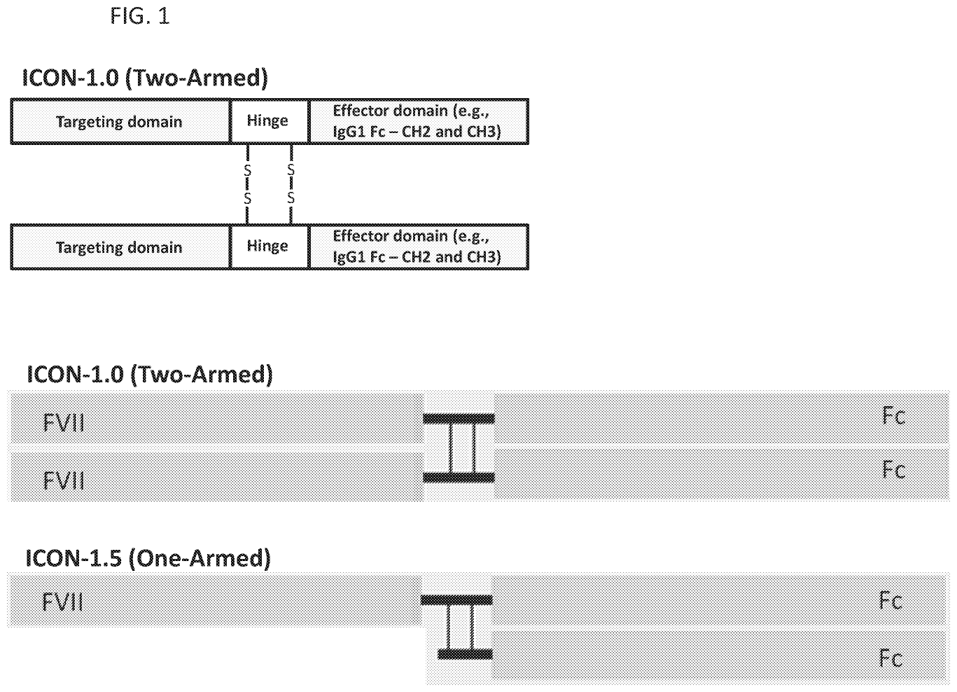

As used herein, “immunoconjugate” or “immunoconjugates” refer to two chemically conjugated or fusion proteins: (1) ICON-1 (referred to interchangeably as hI-con1), a two-armed FVII-Fc fusion protein comprising two dimerized immunoglobulin (Ig) Fc monomers fused to two mutated FVH proteins; and (2) ICON-1.5, a one-armed FVH-Fc fusion protein comprising two dimerized immunoglobulin (Ig) Fc monomers, and a mutated FVH protein, wherein the mutated FVII protein is fused to only one of the Fc monomers. (See FIG. 1 for exemplary embodiments).

As used herein, “conjugated proteins” and “fusion proteins” are used interchangeably, and one of ordinary skill in the art would be aware of the metes and bounds of the distinctions between conjugated proteins and fusion proteins.

In some embodiments, an immunoconjugate has as an effector domain, an immunoglobulin Fc domain, and said effector domain is conjugated to a targeting domain comprising a mutant form of human factor VII. In some embodiments, an immunoconjugate comprises an Fc domain of a human IgG1 immunoglobulin conjugated to a targeting domain exhibiting decreased coagulation comprising a mutant form of factor VII comprising one or two mutations selected from S344A and/or K341A, wherein the immunoconjugate protein binds to tissue factor. In some embodiments, immunoconjugates of the present disclosure include immunoconjugates described in published international patent application WO/2017/181145; and U.S. Pat. Nos. 7,858,092; 8,388,974, 8,071,104; 7,887,809; and 6,924,359.

ICON-1.5 and ICON-1 share similar degrees of binding and ADCC activity, and FXa conversion.

In one aspect provided herein, a protein comprising a mutated FVII protein (targeting domain) conjugated to a human IgG1 Fc region (effector domain) is provided. FIG. 1 provides the generalized structure of one embodiment of an immunoconjugate that can be administered by the methods provided herein. The mutated Factor VIIa domain (also referred to as the TF targeting domain), in the aspects provided herein, binds tissue factor with high affinity and specificity, but does not initiate coagulation, or minimizes coagulation normally associated with tissue factor binding. The IgG1 Fc domain (also referred to as the effector domain) triggers a cytolytic response against cells which bind the immunoconjugate, by the natural killer (NK) cell and complement pathways. In one embodiment, the IgG1 Fc effector domain comprises both the CH2 and CH3 regions of the IgG1 Fc region.

TABLE 1
Description of Sequences

| Sequence Identifier | Description |
|---|---|
| SEQ ID NO: 1 | Homo sapiens factor VII active site mutant |
| | immunoconjugate mRNA, complete CDS; |
| | NCBI Accession AF272774 |
| SEQ ID NO: 2 | Homo sapiens factor VII active site mutant |
| | immunoconjugate amino acid sequence |
| SEQ ID NO: 3 | Homo sapiens factor VII active site mutant |
| | immunoconjugate amino acid sequence, |
| | S344A and A341K (relative to SEQ ID NO: 2) |
| SEQ ID NO: 4 | Homo sapiens factor VII active site mutant |
| | immunoconjugate coding sequence |
| SEQ ID NO: 5 | Homo sapiens factor VII active site mutant |
| | immunoconjugate coding sequence |

The reaction between FVIIa and TF is species-specific (Janson et al., 1984; Schreiber et al., 2005; Peterson et al., 2005): murine FVII appears to be active in many heterologous species including rabbit, pig and human, whereas human FVIIa is only appreciably active in human, dog, rabbit and pig. Conversely, the human IgG Fc domain is active in both humans and mice. Accordingly, depending on the patient, the immunoconjugate is constructed using targeting and effector domains derived from the corresponding species, or from a species that is known to be active in the patient. For example, in the human treatment methods provided herein, the mutated tissue factor targeting domain is derived from human Factor VIIa conjugated to an effector domain comprising the Fc region of a human IgG1 immunoglobulin. For example, in one embodiment, the immunoconjugate is a protein of SEQ ID NO: 2. In a further embodiment, the immunoconjugate is a protein of SEQ ID NO: 3. In one embodiment, the immunoconjugate is encoded by the mRNA sequence of SEQ ID NO: 1, 4, or 5.

In one embodiment, the immunoconjugate described herein comprises two protein chains, each comprising a targeting domain joined to an effector domain via a linker or hinge region. In a further embodiment, the linker or hinge region is naturally occurring, and in one embodiment, is of human origin. The hinge region of an IgG1 immunoglobulin, for example the hinge region of the human IgG1 immunoglobulin, in one embodiment, is used to link the targeting domain to the effector domain. In one embodiment, the hinge region of IgG includes cysteine amino acids which form one or more disulfide bonds between the two monomer chains (e.g., as depicted in FIG. 1).

In one embodiment, the immunoconjugate is a homodimer. However, in another embodiment, the immunoconjugate is a heterodimer, for example, an immunoconjugate comprising two monomers each having a targeting domain of a different amino acid sequence, but the same effector domains. The amino acid sequences of the two targeting domains, in one embodiment, differ by one amino acid, two or more amino acids, three or more amino acids or five or more amino acids. In one embodiment, each monomer subunit comprises an IgG1 hinge region that links the targeting region and effector region of the immunoconjugate, and the monomer subunits of the immunoconjugate heterodimer or the immunoconjugate homodimer are linked together via a disulfide bond between IgG1 hinge regions.

In one embodiment, the molecular weight of the ICON-1 immunoconjugate provided herein is from about 150 kDa to about 200 kDa. In another embodiment, the molecular weight of the immunoconjugate is about 157 kDa or 157 kDa. For example, the immunoconjugate in one embodiment is the immunoconjugate having the amino acid sequence set forth in SEQ ID NO: 2, also referred to herein as “hI-con1” or “ICON-1” (used interchangeably herein). In another embodiment, the immunoconjugate has the amino acid sequence set forth in SEQ ID NO: 3.

As provided throughout, in embodiments described herein, an immunoconjugate comprising a tissue factor targeting domain comprising a mutated Factor VIIa domain is provided. The targeting domain comprises a mutated Factor VIIa that has been mutated to inhibit initiation of the coagulation pathway without reducing binding affinity to tissue factor. In one embodiment, the mutation in Factor VIIa is a single point mutation at residue 341. In a further embodiment, the mutation is from Lys341 to Ala341. However, other mutations that inhibit the coagulation pathway are encompassed by the immunoconjugates provided herein. The effector domain of the immunoconjugates provided herein, in one embodiment, mediates both complement and natural killer (NK) cell cytotoxicity pathways.

Also provided herein are pharmaceutical compositions comprising the immunoconjugates of the invention.

### Immunoconjugate Production

In some embodiments, methods of producing the immunoconjugate include expression in mammalian cells such as BHK cells. In further embodiments, cell lines may include HEK 293, CHO, and SP2/0. Immunoconjugates may be generated by mammalian expression of the expression constructs. In some embodiments, the immunoconjugates are produced as fusion proteins (FVII-Fc) or produced as chemical conjugates.

In some embodiments, the immunoconjugate is post-translationally modified. Post-translational modification includes: myristoylation, glypiation, palmitoylation, prenylation, lipoylation, acylation, alkylation, butrylation, gamma-carboxylation, glycosylation (N-glycosylation, O-glycosylation, fucosylation, and mannosylation), propionylation, succinylation, and sulfation.

### VEGF Inhibitors

As provided herein, the immunoconjugate dimer is administered in a co-therapeutic regimen with a VEGF inhibitor to treat a patient for one of the aforementioned diseases or disorders, for example, to treat wet AMD or another ocular disease associated with angiogenesis or neovascularization.

In one embodiment, the VEGF inhibitor is administered in the same composition as the immunoconjugate dimer.

However, in another embodiment, the immunoconjugate dimer and VEGF inhibitor are administered in separate compositions. In some embodiments, the VEGF inhibitor is administered prior to the immunoconjugate dimer. In some embodiments, the VEGF inhibitor is administered subsequent to the immunoconjugate dimer. In some embodiments, the VEGF inhibitor is administered simultaneously with the immunoconjugate dimer.

In one embodiment, the VEGF inhibitor is an anti-VEGF antibody. In one embodiment, the VEGF inhibitor is ranibizumab or bevacizumab. In a another embodiment, the VEGF in inhibitor is ranibizumab. In another embodiment, ranibizumab is administered at a dosage of 0.5 mg or 0.3 mg per dosing session, and is administered as indicated in the prescribing information for LUCENTIS.

In some embodiments, VEGF inhibitors may be selected from ranibizumab, bevacizumab, pazopanib, sunitinib, sorafenib, axitinib, regorafenib, ponatinib, cabozantinib, vandetanib, ramucirumab, lenvatinib, aflibercept, and ziv-aflibercept.

### Administering the Immunoconjugate and VEGF Inhibitor

Administration methods encompassed by the methods provided herein include intravitreal injection, suprachoroidal injection, topical administration (e.g., eye drops), intravenous and intratumoral administration. In another embodiment, administration is via intravenous, intramuscular, intratumoral, subcutaneous, intrasynovial, intraocular, intraplaque, or intradermal injection of the immunoconjugate or of a replication-deficient adenoviral vector, or other viral vectors carrying a cDNA encoding a secreted form of the immunoconjugate. In one embodiment, the patient in need of treatment is administered one or more immunoconjugate dimers via intravitreal, intravenous or intratumoral injection, or injection at other sites, of one or more immunoconjugate proteins. Alternatively, in one embodiment, a patient in need of treatment is administered one or more immunoconjugate dimers via intravenous or intratumoral injection, or injection at other sites, of one or more expression vectors carrying a cDNA encoding a secreted form of one or more of the immunoconjugate dimers provided herein. In some embodiments, the patient is treated by intravenous or intratumoral injection of an effective amount of one or more replication-deficient adenoviral vectors, or one or more adeno-associated vectors carrying cDNA encoding a secreted form of one or more types of immunoconjugate proteins. In one embodiment, the patient in need of treatment is co-administered one or more immunoconjugate dimers and VEGF inhibitors via intravitreal, intravenous or intratumoral injection, or injection at other sites, of one or more immunoconjugate proteins and VEGF inhibitors. Alternatively, in one embodiment, a patient in need of treatment is co-administered one or more immunoconjugate dimers and VEGF inhibitors via intravenous or intratumoral injection, or injection at other sites, of one or more expression vectors carrying a cDNA encoding a secreted form of one or more of the immunoconjugate dimers provided herein.

As used herein, “effective amount” or “therapeutically effective amount” means a level or amount of a therapeutic agent needed to treat a condition or disease of the present disclosure, or the level or amount of a therapeutic agent that produces a therapeutic response or desired effect in the subject to which the therapeutic agent was administered; wherein a therapeutic agent is an immunoconjugate of the present disclosure. The therapeutic agent may further include an immunoconjugate of the present disclosure and a VEGF inhibitor of the present disclosure. Thus, a therapeutically effective amount of a therapeutic agent, such as an immunoconjugate of the present disclosure and a VEGF inhibitor of the present disclosure, is an amount that is effective in reducing one or more symptoms of angiogenesis and/or neovascularization, as well as various forms of AMD.

As used herein, “pharmaceutical composition” means a composition comprising a therapeutic agent.

As used herein, “treatment”, “treating”, and the like, mean the following actions: (i) preventing a particular disease or disorder from occurring in a subject who may be predisposed to the disease or disorder but has not yet been diagnosed as having it; (ii) curing, treating, or inhibiting the disease, i.e., arresting its development; or (iii) ameliorating or reversing the disease by reducing or eliminating symptoms, conditions, and/or by causing regression of the disease.

In one embodiment, a method of intravitreal injection is employed. In a further embodiment, aseptic technique is employed when preparing the immunoconjugate dimer and/or VEGF inhibitor for injection, for example, via the use of sterile gloves, a sterile drape and a sterile eyelid speculum (or equivalent). In one embodiment, the patient is subjected to anesthesia and a broad-spectrum microbicide prior to the injection.

In one embodiment, intravitreal injection of one or more of the VEGF inhibitors and/or immunoconjugate dimers, provided herein, for example the immunoconjugate dimer of SEQ ID NO: 2 is prepared by withdrawing the vial contents of the immunoconjugate dimer composition solution and/or the VEGF inhibitor composition solution through a 5-micron, 19-gauge filter needle attached to a 1-cc tuberculin syringe. The filter needle in a further embodiment, is then discarded and replaced with a sterile 30-gauge×½-inch needle for the intravitreal injection. The contents of the vial are expelled until the plunger tip is aligned with the line on the syringe that marks the appropriate dose for delivery.

In one method of ocular injection, e.g., intravitreal or suprachoroidal injection, prior to and/or after the injection, the patient is monitored for elevation in intraocular pressure (IOP). For example, in one embodiment, prior to and/or after the ocular injection, the patient is monitored for elevation in IOP using tonometry. In another embodiment, the patient is monitored for increases in IOP via a check for perfusion of the optic nerve head immediately after the injection. In one embodiment, prior to ocular injection of one of the immunoconjugate dimers and/or VEGF inhibitors provided herein, for example about 20 minutes, about 30 minutes, about 40 minutes, about 50 minutes or about 1 hour prior to the ocular injection, the patient is monitored for elevation in IOP. In another embodiment, after ocular injection of one of the immunoconjugate dimers and/or VEGF inhibitors provided herein, for example, about 10 minutes, about 20 minutes, about 30 minutes, about 40 minutes, about 50 minutes or about 1 hour after the intraocular injection, the patient is monitored for elevation in IOP. In one embodiment, the patient's IOP is substantially the same prior to intraocular injection of an immunoconjugate dimer and/or the VEGF inhibitor, as compared to after intraocular injection of the immunoconjugate dimer and/or VEGF inhibitor. In one embodiment, the patient's IOP varies by no more than 10%, no more than 20% or no more than 30% after intraocular injection, as compared to prior to intraocular injection (e.g., intravitreal injection).

The treatment methods provided herein in one embodiment, comprise a single administration of one of the immunoconjugate dimers and/or VEGF inhibitors provided herein (e.g., an immunoconjugate of SEQ ID NO: 2 or 3). However, in another embodiment, the treatment methods provided herein comprise multiple dosing sessions. In a further embodiment, the multiple dosing sessions are multiple intraocular injections of one of the immunoconjugate dimers and/or VEGF inhibitors described herein. The multiple dosing sessions, in one embodiment comprise two or more, three or more, four or more or five or more dosing sessions. In a further embodiment, each dosing session comprises intraocular injection of one of the immunoconjugates and/or VEGF inhibitors described herein, or intratumoral injection of one of the immunoconjugates and/or VEGF inhibitors described herein (i.e., either as the expressed protein or via a vector encoding the soluble immunoconjugate).

In one embodiment, from about 2 to about 24 dosing sessions are employed, for example, from about 2 to about 24 intraocular dosing sessions (e.g., intravitreal or suprachoroidal injection). In a further embodiment, from about 3 to about 30, or from about 5 to about 30, or from about 7 to about 30, or from about 9 to about 30, or from about 10 to about 30, or from about 12 to about 30 or from about 12 to about 24 dosing sessions are employed.

In one embodiment, where multiple dosing sessions are employed, the dosing sessions are spaced apart by from about 10 days to about 60 days, or from about 10 days to about 50 days, or from about 10 days to about 40 days, or from about 10 days to about 30 days, or from about 10 days to about 20 days. In another embodiment, where multiple dosing sessions are employed, the dosing sessions are spaced apart by from about 20 days to about 60 days, or from about 20 days to about 50 days, or from about 20 days to about 40 days, or from about 20 days to about 30 days. In even another embodiment, the multiple dosing sessions are bi-weekly (e.g., about every 14 days), monthly (e.g., about every 30 days), or bi-monthly (e.g., about every 60 days). In yet another embodiment, the dosing sessions are spaced apart by about 28 days.

In one embodiment, the multiple dosing sessions comprise 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 dosing sessions, wherein the dosing sessions are spaced apart by 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 35 days, 40 days, 45 days, 50 days, 55 days, or 60 days.

The immunoconjugates and/or VEGF inhibitors provided herein are amenable for use in any disease or disorder in which angiogenesis and/or neovascularization is implicated. For example, in one aspect, an immunoconjugate dimer and/or VEGF inhibitor provided herein is administered to the eye of a patient in need of treatment of wet age-related macular degeneration (AMD). In one embodiment, the treatment comprises multiple dosing sessions of the immunoconjugate dimer and/or VEGF inhibitors. As provided throughout, the immunoconjugate dimer comprises monomer subunits that each include a mutated human factor VIIa (fVIIa) protein conjugated to the human immunoglobulin G1 (IgG1) Fc domain. In a further embodiment, the immunoconjugate dimer has the amino acid sequence of SEQ ID NO: 2 or 3. In a further embodiment, the immunoconjugate dimer has the amino acid sequence of SEQ ID NO: 3.

In one embodiment, the method of treating wet AMD comprises preventing, inhibiting or reversing choroidal neovascularization in the eye of the patient in need of treatment. In a further embodiment, choroidal neovascularization is reversed by at least about 10%, at least about 20%, at least about 30% or at least about 40% after treatment, as compared to the choroidal neovascularization that was present in the afflicted eye of the patient prior to treatment.

Other ocular disorders associated with ocular neovascularization are treatable with the immunoconjugates and VEGF inhibitors and methods provided herein. The ocular neovascularization, in one embodiment, is choroidal neovascularization. In another embodiment the ocular neovascularization is retinal neovascularization. In yet another embodiment, the ocular neovascularization is corneal neovascularization. Accordingly, an ocular disorder associated with choroidal, retinal or corneal neovascularization, in one embodiment, is treatable by one or more of the methods provided herein. In a further embodiment, the method comprises administering to the eye of a patient in need thereof, the immunoconjugate dimers and/or VEGF inhibitors described herein. In a further embodiment, the treatment comprises multiple dosing sessions of the immunoconjugate dimer and/or VEGF inhibitors. In a further embodiment, the immunoconjugate dimer has the amino acid sequence of SEQ ID NO: 2 or 3. In yet a further embodiment, the immunoconjugate dimer has the amino acid sequence of SEQ ID NO: 2. In a further embodiment, the immunoconjugate dimer has the amino acid sequence of SEQ ID NO: 3.

For example, in one embodiment, a patient in need of treatment of proliferative diabetic retinopathy, wet age-related macular degeneration (AMD), retinopathy of prematurity (ROP), or neovascular glaucoma is treated with the immunoconjugates and/or VEGF inhibitors provided herein, for example, via intravitreal injection, suprachoroidal injection or topical administration (e.g., via eye drops) of the immunoconjugate and VEGF inhibitors into the affected eye. Treatment in one embodiment occurs over multiple dosing sessions. With respect to the aforementioned disorders, ocular neovascularization is said to be “associated with” or “secondary to” the respective disorder.

In one embodiment, a patient in need of treatment of macular edema following retinal vein occlusion (RVO) is treated by one of the immunoconjugate dimers and VEGF inhibitors provided herein. In one embodiment, the method comprises administering to the patient a composition comprising an effective amount of an immunoconjugate dimer and/or VEGF inhibitors, wherein the monomer subunits of the dimer each comprise a mutated factor VIIa (fVIIa) protein conjugated to the human immunoglobulin G1 (IgG1) Fc domain. In a further embodiment, the mutated fVIIa protein is a human mutated fVIIa protein and is linked to the IgG1 Fc domain via the hinge region of IgG1. In a further embodiment, the immunoconjugate dimer has the amino acid sequence of SEQ ID NO: 2 or 3. In yet a further embodiment, the immunoconjugate dimer has the amino acid sequence of SEQ ID NO: 2. In a further embodiment, the immunoconjugate dimer has the amino acid sequence of SEQ ID NO: 3. In one embodiment, the immunoconjugate dimer is administered to the patient over multiple dosing sessions, for example, via intravitreal administration at each dosing session.

In another embodiment, a patient in need of treatment of diabetic macular edema (DME) is treated by one of the immunoconjugate dimers and VEGF inhibitors provided herein. In one embodiment, the method comprises administering to the patient a composition comprising an effective amount of an immunoconjugate dimer and/or VEGF inhibitors, wherein the monomer subunits of the dimer each comprise a mutated factor VIIa (fVIIa) protein conjugated to the human immunoglobulin G1 (IgG1) Fc domain. In a further embodiment, the mutated fVIIa protein is a human mutated fVIIa protein and is linked to the IgG1 Fc domain via the hinge region of IgG1. In a further embodiment, the immunoconjugate dimer has the amino acid sequence of SEQ ID NO: 2 or 3. In yet a further embodiment, the immunoconjugate dimer has the amino acid sequence of SEQ ID NO: 2. In a further embodiment, the immunoconjugate dimer has the amino acid sequence of SEQ ID NO: 3. In one embodiment, the immunoconjugate dimer is administered to the patient over multiple dosing sessions. In even a further embodiment, the immunoconjugate dimer and/or VEGF inhibitor is administered intravitreally at each dosing session.

In yet another embodiment, diabetic retinopathy is treated via one of the immunoconjugates and VEGF inhibitors provided herein, in a patient in need thereof, for example, a patient with DME. In one embodiment, the method comprises administering to the patient, for example a DME patient, a composition comprising an effective amount of an immunoconjugate dimer and/or VEGF inhibitors, wherein the monomer subunits of the dimer each comprise a mutated factor VIIa (fVIIa) protein conjugated to the human immunoglobulin G1 (IgG1) Fc domain. In a further embodiment, the mutated fVIIa protein is a human mutated fVIIa protein and is linked to the IgG1 Fc domain via the hinge region of IgG1. In a further embodiment, the immunoconjugate dimer has the amino acid sequence of SEQ ID NO: 2 or 3. In yet a further embodiment, the immunoconjugate dimer has the amino acid sequence of SEQ ID NO: 2. In a further embodiment, the immunoconjugate dimer has the amino acid sequence of SEQ ID NO: 3. In one embodiment, the immunoconjugate dimer is administered to the patient over multiple dosing sessions. In even a further embodiment, the immunoconjugate dimer and/or VEGF inhibitor is administered to the patient over multiple dosing sessions, for example, via intravitreal administration at each dosing session.

In one embodiment of the invention, one or more of the immunoconjugates and VEGF inhibitors provided herein is used in a method to treat a disease or disorder associated with tumor neovascularization in a patient in need thereof, for example, a cancer patient. In one embodiment, the method comprises administering to the patient, for example via intratumoral or intravenous injection, a composition comprising an effective amount of an immunoconjugate dimer and/or VEGF inhibitor, wherein the monomer subunits of the dimer each comprise a mutated factor VIIa (fVIIa) protein conjugated to the human immunoglobulin G1 (IgG1) Fc domain. In a further embodiment, the mutated fVIIa protein is a human mutated fVIIa protein and is linked to the IgG1 Fc domain via the hinge region of IgG1. In a further embodiment, the immunoconjugate dimer has the amino acid sequence of SEQ ID NO: 2 or 3. In yet a further embodiment, the immunoconjugate dimer has the amino acid sequence of SEQ ID NO: 2. In a further embodiment, the immunoconjugate dimer has the amino acid sequence of SEQ ID NO: 3. In one embodiment, the immunoconjugate dimer and/or VEGF inhibitor is administered to the patient over multiple dosing sessions.

In cancer treatments, the immunoconjugate dimer and VEGF inhibitors are used for treating a variety of cancers, particularly primary or metastatic solid tumors, including melanoma, renal, prostate, breast, ovarian, brain, neuroblastoma, head and neck, pancreatic, bladder, endometrial and lung cancer. In one embodiment, the cancer is a gynecological cancer. In a further embodiment, the gynecological cancer is serous, clear cell, endometriod or undifferentiated ovarian cancer. The immunoconjugate dimer and/or VEGF inhibitor in one embodiment is employed to target the tumor vasculature, particularly vascular endothelial cells, and/or tumor cells. Without wishing to be bound by theory, targeting the tumor vasculature offers several advantages for cancer immunotherapy with one or more of the immunoconjugate dimers and/or VEGF inhibitors described herein, as follows. (i) some of the vascular targets including tissue factor should be the same for all tumors; (ii) immunoconjugates targeted to the vasculature do not have to infiltrate a tumor mass in order to reach their targets; (iii) targeting the tumor vasculature should generate an amplified therapeutic response, because each blood vessel nourishes numerous tumor cells whose viability is dependent on the functional integrity of the vessel; and (iv) the vasculature is unlikely to develop resistance to an immunoconjugate, because that would require modification of the entire endothelium layer lining a vessel. Unlike previously described antiangiogenic methods that inhibit new vascular growth, immunoconjugate dimers provided herein elicit a cytolytic response to the neovasculature.

In another embodiment, one or more of the immunoconjugates and VEGF inhibitors described herein is used in a method for treating atherosclerosis or rheumatoid arthritis. In one embodiment, the method comprises administering to the patient in need of treatment a composition comprising an effective amount of an immunoconjugate dimer and/or VEGF inhibitor, wherein the monomer subunits of the dimer each comprise a mutated factor VIIa (fVIIa) protein conjugated to the human immunoglobulin G1 (IgG1) Fc domain. In a further embodiment, the mutated fVIIa protein is a human mutated fVIIa protein and is linked to the IgG1 Fc domain via the hinge region of IgG. In a further embodiment, the immunoconjugate dimer has the amino acid sequence of SEQ ID NO: 2 or 3. In yet a further embodiment, the immunoconjugate dimer has the amino acid sequence of SEQ ID NO: 2. In a further embodiment, the immunoconjugate dimer has the amino acid sequence of SEQ ID NO: 3. In one embodiment, the immunoconjugate dimer and/or VEGF inhibitor is administered to the patient over multiple dosing sessions.

In one embodiment of a method for treating an ocular disorder with an immunoconjugate dimer and a VEGF inhibitor, for example, a method for treating wet AMD, diabetic retinopathy, diabetic macular edema, or choroidal neovascularization secondary to an ocular disorder such as wet AMD, the patient subjected to the treatment method substantially maintains his or her vision subsequent to the treatment (e.g., the single dosing session or multiple dosing sessions), as measured by losing fewer than 15 letters in a best-corrected visual acuity (BCVA) measurement, compared to the patient's BCVA measurement prior to undergoing treatment. In a further embodiment, the patient loses fewer than 10 letters, fewer than 8 letters, fewer than 6 letters or fewer than 5 letters in a BCVA measurement, compared to the patient's BCVA measurement prior to undergoing treatment.

In some embodiments, a patient having been administered an immunoconjugate and/or VEGF inhibitor of the present invention loses fewer than 10, 9, 8, 7, 6, or 5 letters in a BCVA measurement, compared to a patient's BCVA measurement prior to undergoing treatment. In some embodiments, the patient loses fewer than about 10, about 9, about 8, about 7, about 6, or about 5 letters in a BCVA measurement, compared to a patient's BCVA measurement prior to undergoing treatment.

In some embodiments, a patient having been administered an immunoconjugate and a VEGF inhibitor of the present invention loses fewer than between 15 and 5, 15 and 6, 15 and 7, 15 and 8, 15 and 9, 15 and 10, 10 and 5, 10 and 6, 10 and 7, 10 and 8, 10 and 9, 9 and 5, 9 and 6, 9 and 7, 9 and 8, 8 and 5, 8 and 6, 8 and 7, 7 and 5, 7 and 6, or 6 and 5 letters in a BCVA measurement.

In some embodiments, a patient having been administered an immunoconjugate and a VEGF inhibitor of the present invention loses fewer than between about 15 and about 5, about 15 and about 6, about 15 and about 7, about 15 and about 8, about 15 and about 9, about 15 and about 10, about 10 and about 5, about 10 and about 6, about 10 and about 7, about 10 and about 8, about 10 and about 9, about 9 and about 5, about 9 and about 6, about 9 and about 7, about 9 and about 8, about 8 and about 5, about 8 and about 6, about 8 and about 7, about 7 and about 5, about 7 and about 6, or about 6 and about 5 letters in a BCVA measurement.

In another embodiment of a method for treating an ocular disorder with an immunoconjugate dimer, for example, a method for treating wet AMD, diabetic retinopathy, diabetic macular edema, or choroidal neovascularization secondary to an ocular disorder such as wet AMD, the patient subjected to the treatment method substantially maintains his or her vision subsequent to the treatment (e.g., the single dosing session or multiple dosing sessions), as measured by BCVA measurement.

In some embodiments, a patient having been administered an immunoconjugate and a VEGF inhibitor of the present invention regains his or her vision subsequent to the treatment, as measured by gaining 5, 6, 7, 8, 9, 10, 15, 20, or 25 or more letters in a best-corrected visual acuity (BCVA) measurement, compared to the patient's BCVA prior to the treatment. In some embodiments, a patient having been administered an immunoconjugate and/or VEGF inhibitor of the present invention regains his or her vision subsequent to the treatment, as measured by gaining about 5, about 6, about 7, about 8, about 9, about 10, about 15, about 20, or about 25 or more letters in a best-corrected visual acuity (BCVA) measurement, compared to the patient's BCVA prior to the treatment.

In some embodiments, a patient having been administered an immunoconjugate and a VEGF inhibitor of the present invention regains his or her vision subsequent to the treatment, as measured by gaining greater than between 5 and 25, 5 and 20, 5 and 15, 5 and 10, 5 and 9, 5 and 8, 5 and 7, 5 and 6, 6 and 25, 6 and 20, 6 and 15, 6 and 10, 6 and 9, 6 and 8, 6 and 7, 7 and 25, 7 and 20, 7 and 15, 7 and 10, 7 and 9, 7 and 8, 8 and 25, 8 and 20, 8 and 15, 8 and 10, 8 and 9, 9 and 25, 9 and 20, 9 and 15, 9 and 10, 10 and 25, 10 and 20, 10 and 15, 15 and 25, 15 and 20, or 20 and 25 or more letters in a BCVA measurement, compared to the patient's BCVA prior to the treatment.

In some embodiments, a patient having been administered an immunoconjugate and a VEGF inhibitor of the present invention regains his or her vision subsequent to the treatment, as measured by gaining greater than between about 5 and about 25, about 5 and about 20, about 5 and about 15, about 5 and about 10, about 5 and about 9, about 5 and about 8, about 5 and about 7, about 5 and about 6, about 6 and about 25, about 6 and about 20, about 6 and about 15, about 6 and about 10, about 6 and about 9, about 6 and about 8, about 6 and about 7, about 7 and about 25, about 7 and about 20, about 7 and about 15, about 7 and about 10, about 7 and about 9, about 7 and about 8, about 8 and about 25, about 8 and about 20, about 8 and about 15, about 8 and about 10, about 8 and about 9, about 9 and about 25, about 9 and about 20, about 9 and about 15, about 9 and about 10, about 10 and about 25, about 10 and about 20, about 10 and about 15, about 15 and about 25, about 15 and about 20, or about 20 and about 25 or more letters in a BCVA measurement, compared to the patient's BCVA prior to the treatment.

In one embodiment of a method for treating an ocular disorder with an immunoconjugate dimer and a VEGF inhibitor, for example, a method for treating wet AMD, diabetic retinopathy, diabetic macular edema, or choroidal neovascularization secondary to an ocular disorder such as wet AMD, the ocular neovascularization area, e.g., the choroidal neovascularization area is reduced in the eye of the patient, as compared to the ocular neovascularization area (e.g., CNV area) prior to treatment. As provided herein, treatment can include one dosing session or multiple dosing sessions, and reduction in ocular neovascularization area (e.g., CNV area), in one embodiment, is assessed after individual dosing sessions, or multiple dosing sessions. In a further embodiment, the ocular neovascularization area (e.g., CNV area) is reduced by at least about 5%, or at least about 10%, or at least about 15%, or at least about 20%, or at least about 25%, or at least about 30%, or at least about 35%, or at least about 40%, or at least about 45%, or at least about 50%, as measured by fluorescein angiography.

In one embodiment of a method for treating an ocular disorder with an immunoconjugate dimer and a VEGF inhibitor, for example, a method for treating wet AMD, diabetic retinopathy, diabetic macular edema, or choroidal neovascularization secondary to an ocular disorder such as wet AMD, the retinal thickness of the treated eye is reduced in the eye of the patient, as compared to the retinal thickness prior to treatment, as measured by optical coherence tomography (OCT). As provided herein, treatment can include one dosing session or multiple dosing sessions, and reduction in retinal thickness, in one embodiment, is assessed after individual dosing sessions, or multiple dosing sessions. In a further embodiment, the retinal thickness is reduced by at least about 5%, or at least about 10%, or at least about 15%, or at least about 20%, or at least about 25%, or at least about 30%, or at least about 35%, or at least about 40%, or at least about 45%, or at least about 50%, as measured by OCT. In a further embodiment, the decreased retinal thickness is decreased central retinal subfield thickness (CST), decreased center point thickness (CPT), or decreased central foveal thickness (CFT).

In one embodiment, the immunoconjugate dimer and/or VEGF inhibitor is administered as a solution or a suspension. The immunoconjugate composition and/or VEGF inhibitor composition, in one embodiment, comprises arginine or protein A. In a further embodiment, the immunoconjugate composition and/or VEGF inhibitor composition comprises arginine. In even a further embodiment, the arginine is present in the composition at from about 20 mM to about 40 mM, e.g., at 25 mM. Other components of the composition, in one embodiment, include HEPES, sodium chloride, polysorbate-80, calcium chloride, or a combination thereof.

In one embodiment, the immunoconjugate dimer and/or VEGF inhibitor is administered in a dose of between 10 μg and 600 μg, 10 μg and 500 μg, 10 μg and 400 μg, 10 μg and 300 μg, 10 μg and 200 μg, 10 μg and 100 μg, 10 μg and 50 μg, 50 μg and 600 μg, 50 μg and 500 μg, 50 μg and 400 μg, 50 μg and 300 μg, 50 μg and 200 μg, 50 μg and 100 μg, 100 μg and 600 μg, 100 μg and 500 μg, 100 μg and 400 μg, 100 μg and 300 μg, 100 μg and 200 μg, 200 μg and 600 μg, 200 μg and 500 μg, 200 μg and 400 μg, 200 μg and 300 μg, 300 μg and 600 μg, 300 μg and 500 μg, 300 μg and 400 μg, 400 μg and 600 μg, 400 μg and 500 μg.

In one embodiment, the immunoconjugate dimer and/or VEGF inhibitor is administered in a dose of between about 10 μg and about 500 μg, about 10 μg and about 400 μg, about 10 μg and about 300 μg, about 10 μg and about 200 μg, about 10 μg and about 100 μg, about 10 μg and about 50 μg, about 50 μg and about 500 μg, about 50 μg and about 400 μg, about 50 μg and about 300 μg, about 50 μg and about 200 μg, about 50 μg and about 100 μg, about 100 μg and about 500 μg, about 100 μg and about 400 μg, about 100 μg and about 300 μg, about 100 μg and about 200 μg, about 200 μg and about 500 μg, about 200 μg and about 400 μg, about 200 μg and about 300 μg, about 300 μg and about 500 μg, about 300 μg and about 400 μg, or about 400 μg and about 500 μg.

In one embodiment, the immunoconjugate dimer and/or VEGF inhibitor is administered in a dose of about 0.1 mg, about 0.15 mg, about 0.2 mg, about 0.25 mg, about 0.3 mg, about 0.35 mg, about 0.40 mg, about 0.45 mg, about 0.50 mg, about 0.55 mg, about 0.60 mg, about 0.65 mg, about 0.7 mg, about 0.75 mg, about 0.8 mg, about 0.9 mg, about 0.95 mg, about 1 mg, about 1.05 mg, about 1.1 mg, about 1.15 mg, about 1.2 mg, about 1.25 mg, about 1.30 mg, about 1.35 mg, about 1.4 mg, about 1.45 mg, or about 1.50 mg.

In one embodiment, the immunoconjugate dimer and/or VEGF inhibitor is administered in a dose of 0.1 mg, 0.15 mg, 0.2 mg, 0.25 mg, 0.3 mg, 0.35 mg, 0.40 mg, 0.45 mg, 0.50 mg, 0.55 mg, 0.60 mg, 0.65 mg, 0.7 mg, 0.75 mg, 0.8 mg, 0.9 mg, 0.95 mg, 1 mg, 1.05 mg, 1.1 mg, 1.15 mg, 1.2 mg, 1.25 mg, 1.30 mg, 1.35 mg, 1.4 mg, 1.45 mg, or 1.50 mg.

In one embodiment, the immunoconjugate dimer and/or VEGF inhibitor is administered in a dose consisting of about 10 μg, about 20 μg, about 30 μg, about 40 μg, about 50 μg, about 60 μg, about 70 μg, about 80 μg, about 90 μg, about 100 μg, about 125 μg, about 150 μg, about 175 μg, about 200 μg, about 225 μg, about 250 μg, about 275 μg, about 300 μg, about 325 μg, about 350 μg, about 375 μg, about 400 μg, about 425 μg, about 450 μg, about 475 μg, about 500 μg, about 525 μg, about 550 μg, about 575 μg, about 600 μg, about 625 μg, about 650 μg, about 675 μg, or about 700 μg,

In one embodiment, the immunoconjugate dimer and/or VEGF inhibitor is administered in a solute volume of between 10 μL and 200 μL, 10 μL and 180 μL, 10 μL and 160 μL, 10 μL and 140 μL, 10 μL and 120 μL, 10 μL and 100 μL, 10 μL and 80 μL, 10 μL and 60 μL, 10 μL and 40 μL, 10 μL and 20 μL, 10 μL and 15 μL, 20 μL and 200 μL, 20 μL and 180 μL, 20 μL and 160 μL, 20 μL and 140 μL, 20 μL and 120 μL, 20 μL and 100 μL, 20 μL and 80 μL, 20 μL and 60 μL, 20 μL and 40 μL, 40 μL and 200 μL, 40 μL and 180 μL, 40 μL and 160 μL, 40 μL and 140 μL, 40 μL and 120 μL, 40 μL and 100 μL, 40 μL and 80 μL, 40 μL and 60 μL, 60 μL and 200 μL, 60 μL and 180 μL, 60 μL and 160 μL, 60 μL and 140 μL, 60 μL and 120 μL, 60 μL and 100 μL, 60 μL and 80 μL, 80 μL and 200 μL, 80 μL and 180 μL, 80 μL and 160 μL, 80 μL and 140 μL, 80 μL and 120 μL, 80 μL and 100 μL, 100 μL and 200 μL, 100 μL and 180 μL, 100 μL and 160 μL, 100 μL and 140 μL, 100 μL and 120 μL, 120 μL and 200 μL, 120 μL and 180 μL, 120 μL and 160 μL, 120 μL and 140 μL, 140 μL and 200 μL, 140 μL and 180 μL, 140 μL and 160 μL, 160 μL and 200 μL, 160 μL and 180 μL, or 180 μL and 200 μL.

In one embodiment, the immunoconjugate dimer and/or VEGF inhibitor is administered in a solute volume consisting of about 10 μL, about 15 μL, about 20 μL, about 25 μL, about 30 μL, about 35 μL, about 40 μL, about 45 μL, about 50 μL, about 55 μL, about 60 μL, about 65 μL, about 70 μL, about 75 μL, about 80 μL, about 85 μL, about 90 μL, about 95 μL, or about 100 μL.

One exemplary composition of the present invention is provided in Table 2 below.

TABLE 2
Exemplary immunoconjugate dimer
composition of the invention

| Component | Concentration |
|---|---|
| Immunoconjugate dimer | 3 mg/mL in 15 mM HEPES |
| NaCl | 150 mM |
| Arginine | 25 mM, pH 7.4 |
| Polysorbate-80 | 0.01% |
| CaCl₂ | 5 mM |

### Treatment Outcomes

As provided herein, the administration of an immunoconjugate dimer of the invention and VEGF inhibitor results in an improved outcome compared to administration of the VEGF inhibitor alone. In some embodiments, the improved outcome is greater than an additive effect of the dimer and VEGF inhibitor. In some embodiments, the improved outcome is synergistic as compared to treatment with the dimer or VEGF alone. Outcome can be quantified by BVCA letter score; central subfield retinal thickness; thickness in the tissues/regions of the eye; CNV area, lesion area: CNV-associated exudation; CNV leakage area; volume of sub-retinal fluid; thickness of the central subfield sub-retinal hyper-reflective material, volume of total sub-retinal hyper-reflective material; presence or absence of intraretinal fluid, sub-retinal fluid, and/or sub-retinal pigment epithelium fluid; presence or absence of subfoveal and/or non-subfoveal cysts; atrophy and/or fibrosis; area of autofluorescence; area of discontinuous autofluorescence; volume of the central subfield pigment epithelium detachment; and integrity of the eye's outer nuclear layer, external limiting membrane, ellipsoid zone, and/or subfoveal retinal pigment epithelium.

In some embodiments, a patient is administered, in multiple dosing sessions, an effective amount of (1) an immunoconjugate dimer, wherein the monomer subunits of the dimer each comprises a mutated human factor VIIa (fVIIa) protein conjugated to the human immunoglobulin G1 (IgG1) Fc domain and (2) a VEGF inhibitor. In a further embodiment, the administration, in multiple dosing sessions, of the immunoconjugate dimer and the VEGF inhibitor prevents, inhibits, or reverses wet age-related macular degeneration (AMD) in an eye of a patient in need thereof. In a further embodiment, the administration, in multiple dosing sessions, of the immunoconjugate dimer and the VEGF inhibitor prevents, inhibits, or reverses ocular neovascularization in an eye of a patient in need thereof. In a further embodiment, the administration, in multiple dosing sessions, of the immunoconjugate dimer and the VEGF reverses tumor neovascularization in an eye of a patient in need thereof.

The administration of both an immunoconjugate dimer and a VEGF inhibitor results in superior clinical outcomes as compared to VEGF inhibitor monotherapy, as detailed herein.

In one embodiment, a BCVA letter score is determined in a patient or a population of patients wherein patients are grouped into one of the following three groups: (1) immunoconjugate dimer monotherapy, (2) VEGF inhibitor monotherapy (e.g. anti-VEGF antibody, e.g. Ranibizumab), and (3) immunoconjugate dimer and VEGF inhibitor therapy treatment groups. In some embodiments the BCVA letter score baseline is determined and is then repeated following the commencement of treatment at 1 week, 2 weeks, 3 weeks, 1 month, 2 months, 3 months, 4 months, 5 months, 6 months, 7 months, 8 months, 9 months, 10 months, 11 months, or 12 months. In some embodiments the CNV is classical CNV. In other embodiments, the CNV is occult. In other embodiments, the CNV is minimally classic. In one embodiment, the assessment BVCA letter score determinations occur as a last observation carried forward (LOCF) method.

In some embodiments, a patient gains greater than 5, 10, 15, 20, 25, 30, 35, or 40 letters in the BCVA letter score at 1 month, 2 months, 3 months, 4 months, 5 months, 6 months, 7 months, 8 months, 9 months, 10 months, 11 months, or 12 months after beginning treatment. In some embodiments, a patient gains greater than about 5, about 10, about 15, about 20, about 25, about 30, about 35, or about 40 letters in the BCVA letter score at 1 month, 2 months, 3 months, 4 months, 5 months, 6 months, 7 months, 8 months, 9 months, 10 months, 11 months, or 12 months after beginning treatment.

In some embodiments, a patient treated with an immunoconjugate dimer and a VEGF inhibitor exhibits about a 5%, 10%, 15%, 20%, 25%, 30%, 35%, 40%, 45%, 50%, 55%, 60%, 65%, 70%, 75%, 80%, 85%, 90%, 95%, or even a 100% greater improvement in the BCVA letter score, compared to a patient who received the VEGF inhibitor monotherapy.

In one embodiment, the central subfield retinal thickness in the eye is determined in a patient or a population of patients wherein patients are grouped into one of the following three groups: (1) ICON-1 immunoconjugate dimer monotherapy, (2) VEGF inhibitor monotherapy (e.g. anti-VEGF antibody, e.g. ranibizumab), and (3) immunoconjugate dimer and VEGF inhibitor therapy treatment groups. In some embodiments the central subfield retinal thickness baseline is determined, and is then repeated following the commencement of treatment at 1 week, 2 weeks, 3 weeks, 1 month, 2 months, 3 months, 4 months, 5 months, 6 months, 7 months, 8 months, 9 months, 10 months, 11 months, or 12 months. In some embodiments the CNV is classical CNV. In other embodiments, the CNV is occult. In one embodiment, the central subfield retinal thickness determinations occur as a last observation carried forward (LOCF) method. In one embodiment, the central subfield retinal thickness determination is made utilizing sdOCT.

In some embodiments, a patient exhibits an increase or decrease in the central subfield retinal thickness by at least 5%, 10, 15%, 20%, 25%, 30%, 35%, 40%, 45%, 50%, 55%, 60%, 65%, 70%, 75%, 80%, 85%, 90%, 95%, or 100% at 1 month, 2 months, 3 months, 4 months, 5 months, 6 months, 7 months, 8 months, 9 months, 10 months, 11 months, or 12 months after beginning treatment.

In some embodiments, a patient exhibits an increase or decrease in the central subfield retinal thickness by at least 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% at 1 month, 2 months, 3 months, 4 months, 5 months, 6 months, 7 months, 8 months, 9 months, 10 months, 11 months, or 12 months after beginning treatment.

In one embodiment, the patient exhibits an increase or decrease in the central subfield retinal thickness of the tissues and/or regions of the eye presented herein is an increase or decrease of at least about 10 μm, about 20 μm, about 30 μm, about 40 μm, about 50 μm, about 60 μm, about 70 μm, about 80 μm, about 90 μm, about 100 μm, about 125 μm, about 150 μm, about 175 μm, about 200 μm, about 225 μm, about 250 μm, about 275 μm, about 300 μm, about 325 μm, about 350 μm, about 375 μm, about 400 μm, about 425 μm, about 450 μm, about 475 μm, about 500 μm, about 525 μm, about 550 μm, about 575 μm, about 600 μm, about 625 μm, about 650 μm, about 675 μm, or about 700 μm.

In one embodiment, the measure of thickness of the tissues and/or regions of the eye presented herein is an increase or decrease of at least 10 μm, 20 μm, 30 μm, 40 μm, 50 μm, 60 μm, 70 μm, 80 μm, 90 μm, 100 μm, 125 μm, 150 μm, 175 μm, 200 μm, 225 μm, 250 μm, 275 μm, 300 μm, 325 μm, 350 μm, 375 μm, 400 μm, 425 μm, 450 μm, 475 μm, 500 μm, 525 μm, 550 μm, 575 μm, 600 μm, 625 μm, 650 μm, 675 μm, or 700 μm.

In some embodiments, a patient treated with an immunoconjugate dimer and a VEGF inhibitor exhibits about a 5%, 10%, 15%, 20%, 25%, 30%, 35%, 40%, 45%, 50%, 55%, 60%, 65%, 70%, 75%, 80%, 85%, 90%, 95%, or even a 100% greater increase or decrease in the central subfield retinal thickness, compared to a patient who received the VEGF inhibitor monotherapy.

In one embodiment, a measure of the CNV area is taken in a patient or a population of patients wherein patients are grouped into one of the following three groups: (1) ICON-1 immunoconjugate dimer monotherapy, (2) VEGF inhibitor monotherapy (e.g. anti-VEGF antibody, e.g. ranibizumab), and (3) immunoconjugate dimer and VEGF inhibitor therapy treatment groups. In some embodiments the CNV area baseline is determined and is then repeated following the commencement of treatment at 1 week, 2 weeks, 3 weeks, 1 month, 2 months, 3 months, 4 months, 5 months, 6 months, 7 months, 8 months, 9 months, 10 months, 11 months, or 12 months. In some embodiments the CNV is classical CNV. In other embodiments, the CNV is occult CNV. In one embodiment, the determinations of the CNV areas occur as a last observation carried forward (LOCF) method.

In some embodiments, a patient exhibits a decrease in the CNV area by at least 5%, 10%, 15%, 20%, 25%, 30%, 35%, 40%, 45%, 50%, 55%, 60%, 65%, 70%, 75%, 80%, 85%, 90%, 95%, or 100% at 1 month, 2 months, 3 months, 4 months, 5 months, 6 months, 7 months, 8 months, 9 months, 10 months, 11 months, or 12 months after beginning treatment.

In some embodiments, a patient exhibits a decrease in the CNV area by at least about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% at 1 month, 2 months, 3 months, 4 months, 5 months, 6 months, 7 months, 8 months, 9 months, 10 months, 11 months, or 12 months after beginning treatment.

In some embodiments, a patient treated with an immunoconjugate dimer and a VEGF inhibitor exhibits about a 5%, 10%, 15%, 20%, 25%, 30%, 35%, 40%, 45%, 50%, 55%, 60%, 65%, 70%, 75%, 80%, 85%, 90%, 95%, or even a 100% greater decrease in the CNV area, compared to a patient who received the VEGF inhibitor monotherapy.

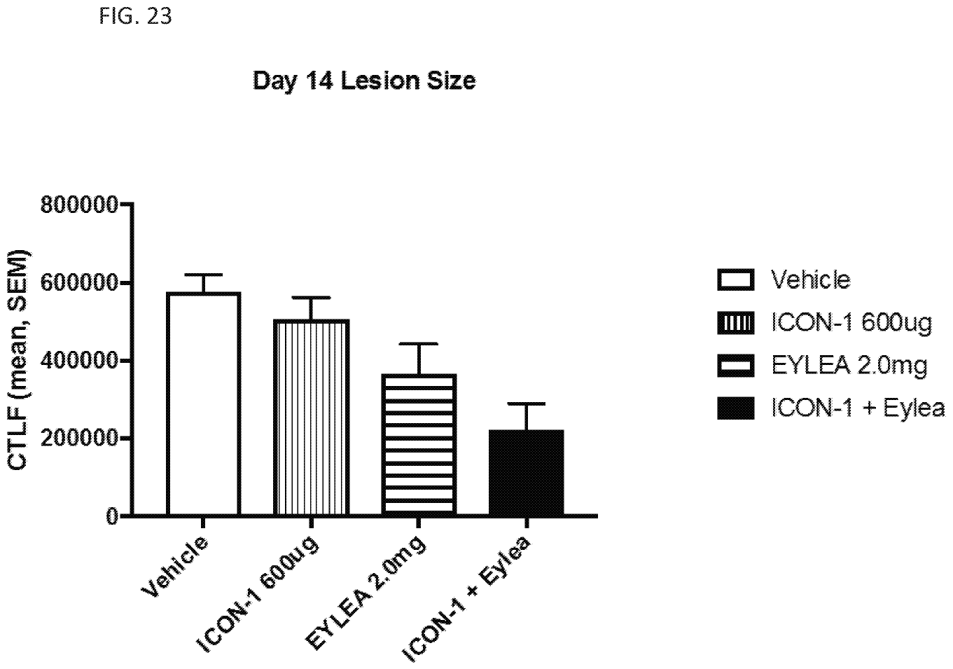

In one embodiment, a measure of the area of the lesion is taken in a patient or a population of patients wherein patients are grouped into one of the following three groups: (1) ICON-1 immunoconjugate dimer monotherapy, (2) VEGF inhibitor monotherapy (e.g. anti-VEGF antibody, e.g. ranibizumab), and (3) immunoconjugate dimer and VEGF inhibitor therapy treatment groups. In some embodiments the lesion area baseline is determined and is then repeated following the commencement of treatment at 1 week, 2 weeks, 3 weeks, 1 month, 2 months, 3 months, 4 months, 5 months, 6 months, 7 months, 8 months, 9 months, 10 months, 11 months, or 12 months. In some embodiments the CNV is classical CNV. In other embodiments, the CNV is occult CNV. In one embodiment, the determinations of the area of lesions occur as a last observation carried forward (LOCF) method. See FIG. 23 for synergistic decrease of lesion size, as measured by CTLF for combination administration of ICON-1+VEGF inhibitor.

In some embodiments, a patient exhibits a decrease in the area of lesions by at least 5%, 10%, 15%, 20%, 25%, 30%, 35%, 40%, 45%, 50%, 55%, 600%, 65%, 70%, 75%, 80%, 85%, 90%, 95%, or 100% at 1 month, 2 months, 3 months, 4 months, 5 months, 6 months, 7 months, 8 months, 9 months, 10 months, 11 months, or 12 months after beginning treatment.

In some embodiments, a patient exhibits a decrease in the area of lesions by at least about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% at 1 month, 2 months, 3 months, 4 months, 5 months, 6 months, 7 months, 8 months, 9 months, 10 months, 11 months, or 12 months after beginning treatment.

In some embodiments, a patient treated with an immunoconjugate dimer and a VEGF inhibitor exhibits about a 5%, 10%, 15%, 20%, 25%, 30%, 35%, 40%, 45%, 50%, 55%, 60%, 65%, 70%, 75%, 80%, 85%, 90%, 95%, or even a 100% greater in the area of lesions, compared to a patient who received the VEGF inhibitor monotherapy.

In one embodiment, a measure of the CNV-associated exudation is taken in a patient or a population of patients wherein patients are grouped into one of the following three groups: (1) ICON-1 immunoconjugate dimer monotherapy, (2) VEGF inhibitor monotherapy (e.g. anti-VEGF antibody, e.g. ranibizumab), and (3) immunoconjugate dimer and VEGF inhibitor therapy treatment groups. In some embodiments the CNV-associated exudation baseline is determined and is then repeated following the commencement of treatment at 1 week, 2 weeks, 3 weeks, 1 month, 2 months, 3 months, 4 months, 5 months, 6 months, 7 months, 8 months, 9 months, 10 months, 11 months, or 12 months. In some embodiments the CNV-associated exudation is classical CNV. In other embodiments, the CNV is occult CNV. In one embodiment, the determinations of the CNV areas occur as a last observation carried forward (LOCF) method.

In some embodiments, a patient exhibits a decrease in the CNV-associated exudation by at least 5%, 10%, 15%, 20%, 25%, 30%, 35%, 40%, 45%, 50%, 55%, 60%, 65%, 70%, 75%, 80%, 85%, 90%, 95%, or 100% at 1 month, 2 months, 3 months, 4 months, 5 months, 6 months, 7 months, 8 months, 9 months, 10 months, 11 months, or 12 months after beginning treatment.

In some embodiments, a patient exhibits a decrease in the CNV-associated exudation by at least at least about 5%, about 100%, about 15%, about 20%, about 25%, about 30%, about 35%, about 40%, about 45%, about 50%, about 55%, about 60%, about 65%, about 70%, about 75%, about 80, about 85%, about 90%, about 95%, or about 100% at 1 month, 2 months, 3 months, 4 months, 5 months, 6 months, 7 months, 8 months, 9 months, 10 months, 11 months, or 12 months after beginning treatment.

In some embodiments, a patient treated with an immunoconjugate dimer and a VEGF inhibitor exhibits about a 5%, 100%, 15%, 20%, 25%, 30%, 35%, 40%, 45%, 50%, 55%, 60%, 65%, 70%, 75%, 80%, 85%, 90%, 95%, or even a 100% greater improvement in the BCVA letter score, compared to a patient who received the VEGF inhibitor monotherapy.

In one embodiment, a measure of the area of leaking CNV (leakage area) is taken in a patient or a population of patients wherein patients are grouped into one of the following three groups: (1) ICON-1 immunoconjugate dimer monotherapy, (2) VEGF inhibitor monotherapy (e.g. anti-VEGF antibody, e.g. ranibizumab), and (3) immunoconjugate dimer and VEGF inhibitor therapy treatment groups. In some embodiments the leakage area baseline is determined and is then repeated following the commencement of treatment at 1 week, 2 weeks, 3 weeks, 1 month, 2 months, 3 months, 4 months, 5 months, 6 months, 7 months, 8 months, 9 months, 10 months, 11 months, or 12 months after beginning treatment. In some embodiments the CNV is classical CNV. In other embodiments, the CNV is occult CNV. In one embodiment, the measure of the leakage area occurs as a last observation carried forward (LOCF) method.

In some embodiments, a patient exhibits a decrease in the area of leakage area by at least 5%, 10%, 15%, 20%, 25%, 30%, 35%, 40%, 45%, 50%, 55%, 60%, 65%, 70%, 75%, 80%, 85%, 90%, 95%, or 100% at 1 month, 2 months, 3 months, 4 months, 5 months, 6 months, 7 months, 8 months, 9 months, 10 months, 11 months, or 12 months after beginning treatment. In some embodiments the CNV is classical CNV. In other embodiments, the CNV is occult.

In some embodiments, a patient exhibits a decrease in the area of leakage area by at least about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% at 1 month, 2 months, 3 months, 4 months, 5 months, 6 months, 7 months, 8 months, 9 months, 10 months, 11 months, or 12 months after beginning treatment.

In some embodiments, a patient treated with an immunoconjugate dimer and a VEGF inhibitor exhibits about a 5%, 10%, 15%, 20%, 25%, 30%, 35%, 40%, 45%, 50%, 55%, 60%, 65%, 70%, 75%, 80%, 85%, 90%, 95%, or even a 100% greater decrease in the area of leakage, compared to a patient who received the VEGF inhibitor monotherapy.

In one embodiment, a measure of the volume of the sub-retinal fluid is taken in a patient or a population of patients wherein patients are grouped into one of the following three groups: (1) ICON-1 immunoconjugate dimer monotherapy, (2) VEGF inhibitor monotherapy (e.g. anti-VEGF antibody, e.g. ranibizumab), and (3) immunoconjugate dimer and VEGF inhibitor therapy treatment groups. In some embodiments the measure of the volume of the sub-retinal fluid baseline is determined and is then repeated following the commencement of treatment at 1 week, 2 weeks, 3 weeks, 1 month, 2 months, 3 months, 4 months, 5 months, 6 months, 7 months, 8 months, 9 months, 10 months, 11 months, or 12 months after beginning treatment. In some embodiments the CNV is classical CNV. In other embodiments, the CNV is occult CNV. In one embodiment, the measure of the measure of the volume of the sub-retinal fluid occurs as a last observation carried forward (LOCF) method.

In some embodiments, a patient exhibits a decrease or increase in the volume of the sub-retinal fluid by at least 5%, 10%, 15%, 20%, 25%, 30%, 35%, 40%, 45%, 50%, 55%, 60%, 65%, 70%, 75%, 80%, 85%, 90%, 95%, or 100% at 1 month, 2 months, 3 months, 4 months, 5 months, 6 months, 7 months, 8 months, 9 months, 10 months, 11 months, or 12 months after beginning treatment. In some embodiments the CNV is classical CNV. In other embodiments, the CNV is occult CNV.

In some embodiments, a patient exhibits a decrease or increase in the volume of the sub-retinal fluid by at least about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% at 1 month, 2 months, 3 months, 4 months, 5 months, 6 months, 7 months, 8 months, 9 months, 10 months, 11 months, or 12 months after beginning treatment.

In some embodiments, a patient treated with an immunoconjugate dimer and a VEGF inhibitor exhibits about a 5%, 10%, 15%, 20%, 25%, 30%, 35%, 40%, 45%, 50%, 55%, 60%, 65%, 70%, 75%, 80%, 85%, 90%, 95%, or even a 100% greater increase or decrease in the volume of the sub-retinal fluid, compared to a patient who received the VEGF inhibitor monotherapy.

In one embodiment, a measure of the thickness of the central subfield subretinal hyper-reflective material is taken in a patient or a population of patients wherein patients are grouped into one of the following three groups: (1) ICON-1 immunoconjugate dimer monotherapy, (2) VEGF inhibitor monotherapy (e.g. anti-VEGF antibody, e.g. ranibizumab), and (3) immunoconjugate dimer and VEGF inhibitor therapy treatment groups. In some embodiments the measure of the thickness of the central subfield subretinal hyper-reflective material baseline is determined and is then repeated following the commencement of treatment at 1 week, 2 weeks, 3 weeks, 1 month, 2 months, 3 months, 4 months, 5 months, 6 months, 7 months, 8 months, 9 months, 10 months, 11 months, or 12 months after beginning treatment. In some embodiments the CNV is classical CNV. In other embodiments, the CNV is occult CNV. In one embodiment, the measure of the thickness of the central subfield subretinal hyper-reflective material occurs as a last observation carried forward (LOCF) method.

In some embodiments, a patient exhibits a decrease or increase in the thickness of the central subfield subretinal hyper-reflective material by at least 5%, 10%, 15%, 20%, 25%, 30%, 35%, 40%, 45%, 50%, 55%, 60%, 65%, 70%, 75%, 80%, 85%, 90%, 95%, or 100% at 1 month, 2 months, 3 months, 4 months, 5 months, 6 months, 7 months, 8 months, 9 months, 10 months, 11 months, or 12 months after beginning treatment. In some embodiments the CNV is classical CNV. In other embodiments, the CNV is occult CNV.

In some embodiments, a patient exhibits a decrease or increase in the thickness of the central subfield subretinal hyper-reflective material by at least about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% at 1 month, 2 months, 3 months, 4 months, 5 months, 6 months, 7 months, 8 months, 9 months, 10 months, 11 months, or 12 months after beginning treatment.

In some embodiments, a patient treated with an immunoconjugate dimer and a VEGF inhibitor exhibits about a 5%, 10%, 15%, 20%, 25%, 30%, 35%, 40%, 45%, 50%, 55%, 60%, 65%, 70%, 75%, 80%, 85%, 90%, 95%, or even a 100% greater increase or decrease in the thickness of the central subfield subretinal hyper-reflective material, compared to a patient who received the VEGF inhibitor monotherapy.

In one embodiment, a measure of the total subretinal hyper-reflective material volume is taken in a patient or a population of patients wherein patients are grouped into one of the following three groups: (1) ICON-1 immunoconjugate dimer monotherapy, (2) VEGF inhibitor monotherapy (e.g. anti-VEGF antibody, e.g. ranibizumab), and (3) immunoconjugate dimer and VEGF inhibitor therapy treatment groups. In some embodiments the measure of the total volume of the subretinal hyper-reflective material baseline is determined and is then repeated following the commencement of treatment at 1 week, 2 weeks, 3 weeks, 1 month, 2 months, 3 months, 4 months, 5 months, 6 months, 7 months, 8 months, 9 months, 10 months, 11 months, or 12 months after beginning treatment. In some embodiments, distinctions are made between subfoveal versus non-subfoveal. In some embodiments the CNV is classical CNV. In other embodiments, the CNV is occult CNV. In one embodiment, the measure of the total volume of the subretinal hyper-reflective material occurs as a last observation carried forward (LOCF) method.

In some embodiments, a patient exhibits a decrease or increase in the total volume of the subretinal hyper-reflective material by at least 5%, 10%, 15%, 20%, 25%, 30%, 35%, 40%, 45%, 50%, 55%, 60%, 65%, 70%, 75%, 80%, 85%, 90%, 95%, or 100% at 1 month, 2 months, 3 months, 4 months, 5 months, 6 months, 7 months, 8 months, 9 months, 10 months, 11 months, or 12 months after beginning treatment. In some embodiments the CNV is classical CNV. In other embodiments, the CNV is occult CNV. In some embodiments, distinctions are made between subfoveal versus non-subfoveal.

In some embodiments, a patient exhibits a decrease or increase in the total volume of the subretinal hyper-reflective material by at least about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% at 1 month, 2 months, 3 months, 4 months, 5 months, 6 months, 7 months, 8 months, 9 months, 10 months, 11 months, or 12 months after beginning treatment. In some embodiments, distinctions are made between subfoveal versus non-subfoveal.

In some embodiments, a patient treated with an immunoconjugate dimer and a VEGF inhibitor exhibits about a 5%, 10%, 15%, 20%, 25%, 30%, 35%, 40%, 45%, 50%, 55%, 60%, 65%, 70%, 75%, 80%, 85%, 90%, 95%, or even a 100% greater increase or decrease in the total volume of the subretinal hyper-reflective material, compared to a patient who received the VEGF inhibitor monotherapy.

In one embodiment, the identification of the presence or absence is made for (1) intraretinal fluid, (2) subretinal fluid, (3) subretinal pigment epithelium fluid in a patient or a population of patients wherein patients are grouped into one of the following three groups: (1) ICON-1 immunoconjugate dimer monotherapy, (2) VEGF inhibitor monotherapy (e.g. anti-VEGF antibody, e.g. ranibizumab), and (3) immunoconjugate dimer and VEGF inhibitor therapy treatment groups. In some embodiments the baseline determination of the presence or absence of fluid in said ocular locations is determined and is then repeated following the commencement of treatment at 1 week, 2 weeks, 3 weeks, 1 month, 2 months, 3 months, 4 months, 5 months, 6 months, 7 months, 8 months, 9 months, 10 months, 11 months, or 12 months after beginning treatment. In some embodiments the CNV is classical CNV. In other embodiments, the CNV is occult CNV. In one embodiment, the determination of the presence or absence of fluid in said ocular locations occurs as a last observation carried forward (LOCF) method.

In some embodiments, a patient exhibits a presence or absence of (1) intraretinal fluid, (2) subretinal fluid, and/or (3) subretinal pigment epithelium fluid at 1 month, 2 months, 3 months, 4 months, 5 months, 6 months, 7 months, 8 months, 9 months, 10 months, 11 months, or 12 months after beginning treatment.

In some embodiments, a patient exhibits a presence or absence of (1) intraretinal fluid, (2) subretinal fluid, and/or (3) subretinal pigment epithelium fluid at 1 month, 2 months, 3 months, 4 months, 5 months, 6 months, 7 months, 8 months, 9 months, 10 months, 11 months, or 12 months after beginning treatment.

In some embodiments, a patient treated with an immunoconjugate dimer and a VEGF inhibitor exhibits about a 5%, 10%, 15%, 20%, 25%, 30%, 35%, 40%, 45%, 50%, 55%, 60%, 65%, 70%, 75%, 80%, 85%, 90%, 95%, or even a 100% greater increase or decrease of (1) intraretinal fluid, (2) subretinal fluid, and/or (3) subretinal pigment epithelium fluid, compared to a patient who received the VEGF inhibitor monotherapy.

In one embodiment, the identification of the presence or absence of subfoveal or non-subfoveal cysts in a patient or a population of patients wherein patients are grouped into one of the following three groups: (1) ICON-1 immunoconjugate dimer monotherapy, (2) VEGF inhibitor monotherapy (e.g. anti-VEGF antibody, e.g. ranibizumab), and (3) immunoconjugate dimer and VEGF inhibitor therapy treatment groups. In some embodiments the baseline determination of the presence or absence of subfoveal or non-subfoveal cysts is determined and is then repeated following the commencement of treatment at 1 week, 2 weeks, 3 weeks, 1 month, 2 months, 3 months, 4 months, 5 months, 6 months, 7 months, 8 months, 9 months, 10 months, 11 months, or 12 months after beginning treatment. In some embodiments the CNV is classical CNV. In other embodiments, the CNV is occult CNV. In one embodiment, the determination of the presence or absence of said cysts occurs as a last observation carried forward (LOCF) method.

In some embodiments, a patient exhibits a presence or absence of subfoveal or non-subfoveal cysts at 1 month, 2 months, 3 months, 4 months, 5 months, 6 months, 7 months, 8 months, 9 months, 10 months, 11 months, or 12 months after beginning treatment. In some embodiments, a patient exhibits a decrease in the presence of subfoveal or non-subfoveal cysts at 1 month, 2 months, 3 months, 4 months, 5 months, 6 months, 7 months, 8 months, 9 months, 10 months, 11 months, or 12 months after beginning treatment.

In some embodiments, a patient exhibits a presence or absence of subfoveal or non-subfoveal cysts at 1 month, 2 months, 3 months, 4 months, 5 months, 6 months, 7 months, 8 months, 9 months, 10 months, 11 months, or 12 months after beginning treatment. In some embodiments, a patient exhibits a decrease in the presence of subfoveal or non-subfoveal cysts at 1 month, 2 months, 3 months, 4 months, 5 months, 6 months, 7 months, 8 months, 9 months, 10 months, 11 months, or 12 months after beginning treatment.

In some embodiments, a patient treated with an immunoconjugate dimer and a VEGF inhibitor exhibits the presence or absence of or about a 5%, 10%, 15%, 20%, 25%, 30%, 35%, 40%, 45%, 50%, 55%, 60%, 65%, 70%, 75%, 80%, 85%, 90%, 95%, or even a 100% decrease in subfoveal or non-subfoveal cysts, compared to a patient who received the VEGF inhibitor monotherapy.

In one embodiment, the identification of atrophy and/or fibrosis is made for the eye in a patient or a population of patients wherein patients are grouped into one of the following three groups: (1) ICON-1 immunoconjugate dimer monotherapy, (2) VEGF inhibitor monotherapy (e.g. anti-VEGF antibody, e.g. ranibizumab), and (3) immunoconjugate dimer and VEGF inhibitor therapy treatment groups. In some embodiments the baseline identification of atrophy and/or fibrosis is determined and is then repeated following the commencement of treatment at 1 week, 2 weeks, 3 weeks, 1 month, 2 months, 3 months, 4 months, 5 months, 6 months, 7 months, 8 months, 9 months, 10 months, 11 months, or 12 months after beginning treatment. In some embodiments the CNV is classical CNV. In other embodiments, the CNV is occult CNV. In one embodiment, the determination of the presence or atrophy and/or fibrosis occurs as a last observation carried forward (LOCF) method.

In some embodiments, a patient exhibits a decrease in the atrophy and/or fibrosis of the eye by at least 5%, 10%, 15%, 20%, 25%, 30%, 35%, 40%, 45%, 50%, 55%, 60%, 65%, 70%, 75%, 80%, 85%, 90%, 95%, or 100% at 1 month, 2 months, 3 months, 4 months, 5 months, 6 months, 7 months, 8 months, 9 months, 10 months, 11 months, or 12 months after beginning treatment.

In some embodiments, a patient exhibits a decrease in the atrophy and/or fibrosis of the eye by at least about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% at 1 month, 2 months, 3 months, 4 months, 5 months, 6 months, 7 months, 8 months, 9 months, 10 months, 11 months, or 12 months after beginning treatment.

In some embodiments, a patient treated with an immunoconjugate dimer and a VEGF inhibitor exhibits about a 5%, 10%, 15%, 20%, 25%, 30%, 35%, 40%, 45%, 50%, 55%, 60%, 65%, 70%, 75%, 80%, 85%, 90%, 95%, or even a 100% greater decrease in the atrophy and/or fibrosis of the eye, compared to a patient who received the VEGF inhibitor monotherapy.

In one embodiment, the total area of decreased autofluorescence is determined for the in a patient or a population of patients wherein patients are grouped into one of the following three groups: (1) ICON-1 immunoconjugate dimer monotherapy, (2) VEGF inhibitor monotherapy (e.g. anti-VEGF antibody, e.g. ranibizumab), and (3) immunoconjugate dimer and VEGF inhibitor therapy treatment groups. In some embodiments, the baseline determination of the area of decreased autofluorescence is determined and is then repeated following the commencement of treatment at 1 week, 2 weeks, 3 weeks, 1 month, 2 months, 3 months, 4 months, 5 months, 6 months, 7 months, 8 months, 9 months, 10 months, 11 months, or 12 months after beginning treatment. In some embodiments the CNV is classical CNV. In other embodiments, the CNV is occult CNV. In one embodiment, the determination of the total area of decreased autofluorescence occurs as a last observation carried forward (LOCF) method.

In some embodiments, a patient exhibits a decrease in the total area of autofluorescence the eye by at least 5%, 10%, 15%, 20%, 25%, 30%, 35%, 40%, 45%, 50%, 55%, 60%, 65%, 70%, 75%, 80%, 85%, 90%, 95%, or 100% at 1 month, 2 months, 3 months, 4 months, 5 months, 6 months, 7 months, 8 months, 9 months, 10 months, 11 months, or 12 months after beginning treatment.

In some embodiments, a patient exhibits a decrease in the total area of autofluorescence the eye by at least about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% at 1 month, 2 months, 3 months, 4 months, 5 months, 6 months, 7 months, 8 months, 9 months, 10 months, 11 months, or 12 months after beginning treatment.

In some embodiments, a patient treated with an immunoconjugate dimer and a VEGF inhibitor exhibits about a 5%, 10%, 15%, 20%, 25%, 30%, 35%, 40%, 45%, 50%, 55%, 60%, 65%, 70%, 75%, 80%, 85%, 90%, 95%, or even a 100% greater decrease in the total area of autofluorescence, compared to a patient who received the VEGF inhibitor monotherapy.

In one embodiment, the total area of discontinuous autofluorescence in the eye is determined for a patient or a population of patients wherein patients are grouped into one of the following three groups: (1) ICON-1 immunoconjugate dimer monotherapy, (2) VEGF inhibitor monotherapy (e.g. anti-VEGF antibody, e.g. ranibizumab), and (3) immunoconjugate dimer and VEGF inhibitor therapy treatment groups. In some embodiments, the baseline determination of the total area of discontinuous autofluorescence is determined and is then repeated following the commencement of treatment at 1 week, 2 weeks, 3 weeks, 1 month, 2 months, 3 months, 4 months, 5 months, 6 months, 7 months, 8 months, 9 months, 10 months, 11 months, or 12 months after beginning treatment. In some embodiments the CNV is classical CNV. In other embodiments, the CNV is occult CNV. In one embodiment, the determination of the total area of discontinuous autofluorescence occurs as a last observation carried forward (LOCF) method.

In some embodiments, a patient exhibits a decrease in the total area of discontinuous autofluorescence the eye by at least 5%, 10%, 15%, 20%, 25%, 30%, 35%, 40%, 45%, 50%, 55%, 60%, 65%, 70%, 75%, 80%, 85%, 90%, 95%, or 100% at 1 month, 2 months, 3 months, 4 months, 5 months, 6 months, 7 months, 8 months, 9 months, 10 months, 11 months, or 12 months after beginning treatment.

In some embodiments, a patient exhibits a decrease in the total area of discontinuous autofluorescence the eye by at least about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% at 1 month, 2 months, 3 months, 4 months, 5 months, 6 months, 7 months, 8 months, 9 months, 10 months, 11 months, or 12 months after beginning treatment.

In some embodiments, a patient treated with an immunoconjugate dimer and a VEGF inhibitor exhibits about a 5%, 10%, 15%, 20%, 25%, 30%, 35%, 40%, 45%, 50%, 55%, 60%, 65%, 70%, 75%, 80%, 85%, 90%, 95%, or even a 100% greater decrease in the total area of discontinuous autofluorescence, compared to a patient who received the VEGF inhibitor monotherapy.

In one embodiment, a measurement of the volume of the central subfield pigment epithelium detachment is determined for in a patient or a population of patients wherein patients are grouped into one of the following three groups: (1) ICON-1 immunoconjugate dimer monotherapy, (2) VEGF inhibitor monotherapy (e.g. anti-VEGF antibody, e.g. ranibizumab), and (3) immunoconjugate dimer and VEGF inhibitor therapy treatment groups. In some embodiments, the baseline determination of the volume of the central subfield pigment epithelium detachment is determined and is then repeated following the commencement of treatment at 1 week, 2 weeks, 3 weeks, 1 month, 2 months, 3 months, 4 months, 5 months, 6 months, 7 months, 8 months, 9 months, 10 months, 11 months, or 12 months after beginning treatment. In some embodiments the CNV is classical CNV. In other embodiments, the CNV is occult CNV. In one embodiment, the volume of the central subfield pigment epithelium detachment occurs as a last observation carried forward (LOCF) method.

In some embodiments, a patient exhibits a decrease in the volume of the central subfield pigment epithelium detachment by at least 5%, 10%, 15%, 20%, 25%, 30%, 35%, 40%, 45%, 50%, 55%, 60%, 65%, 70%, 75%, 80%, 85%, 90%, 95%, or 100% at 1 month, 2 months, 3 months, 4 months, 5 months, 6 months, 7 months, 8 months, 9 months, 10 months, 11 months, or 12 months after beginning treatment.

In some embodiments, a patient exhibits a decrease in the volume of the central subfield pigment epithelium detachment by at least about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% at 1 month, 2 months, 3 months, 4 months, 5 months, 6 months, 7 months, 8 months, 9 months, 10 months, 11 months, or 12 months after beginning treatment.

In some embodiments, a patient treated with an immunoconjugate dimer and a VEGF inhibitor exhibits about a 5%, 10%, 15%, 20%, 25%, 30%, 35%, 40%, 45%, 50%, 55%, 60%, 65%, 70%, 75%, 80%, 85%, 90%, 95%, or even a 100% greater decrease in the volume of the central subfield pigment epithelium detachment, compared to a patient who received the VEGF inhibitor monotherapy.

In one embodiment, a determination of the integrity of the (1) outer nuclear layer, (2) external limiting membrane, (3) ellipsoid zone, and (4) subfoveal retinal pigment epithelium of an eye is made for a patient or a population of patients wherein patients are grouped into one of the following three groups: (1) ICON-1 immunoconjugate dimer monotherapy, (2) VEGF inhibitor monotherapy (e.g. anti-VEGF antibody, e.g. ranibizumab), and (3) immunoconjugate dimer and VEGF inhibitor therapy treatment groups. In some embodiments, the baseline determination of the integrity of (1)-(4) is determined and is then repeated following the commencement of treatment at 1 week, 2 weeks, 3 weeks, 1 month, 2 months, 3 months, 4 months, 5 months, 6 months, 7 months, 8 months, 9 months, 10 months, 11 months, or 12 months after beginning treatment. In some embodiments the CNV is classical CNV. In other embodiments, the CNV is occult CNV. In one embodiment, the determination of the integrity of (10-(4) occurs as a last observation carried forward (LOCF) method.

In some embodiments, a patient exhibits an increase in the integrity of the (1) outer nuclear layer, (2) external limiting membrane, (3) ellipsoid zone, and (4) subfoveal retinal pigment epithelium of the eye by at least 5%, 10%, 15%, 20%, 25%, 30%, 35%, 40%, 45%, 50%, 55%, 60%, 65%, 70%, 75%, 80%, 85%, 90%, 95%, or 100% at 1 month, 2 months, 3 months, 4 months, 5 months, 6 months, 7 months, 8 months, 9 months, 10 months, 11 months, or 12 months after beginning treatment.

In some embodiments, a patient exhibits an increase in the integrity of the (1) outer nuclear layer, (2) external limiting membrane, (3) ellipsoid zone, and (4) subfoveal retinal pigment epithelium of the eye by at least about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% at 1 month, 2 months, 3 months, 4 months, 5 months, 6 months, 7 months, 8 months, 9 months, 10 months, 11 months, or 12 months after beginning treatment.

In some embodiments, a patient treated with an immunoconjugate dimer and a VEGF inhibitor exhibits about a 5%, 10%, 15%, 20%, 25%, 30%, 35%, 40%, 45%, 50%, 55%, 60%, 65%, 70%, 75%, 80%, 85%, 90%, 95%, or even a 100% greater increase in the integrity of the (1) outer nuclear layer, (2) external limiting membrane, (3) ellipsoid zone, and (4) subfoveal retinal pigment epithelium of the eye, compared to a patient who received the VEGF inhibitor monotherapy.

In some embodiments, the immunoconjugate dimer utilized in treatments of the present disclosure is the ICON-1.5 immunoconjugate.

### EXAMPLES

The present invention is further illustrated by reference to the following Examples. However, it should be noted that these Examples, like the embodiments described above, are illustrative and are not to be construed as restricting the scope of the invention in any way.

### Example 1—Evaluation of hI-con1 in In Vitro Thrombin Generation Assays

The effect of hI-con1 (SEQ ID NO:2) in a thrombin generation assay in plasma was tested. Specifically, the effect of hI-con1 on thrombin generation in plasma in a tissue factor initiated reaction using a thrombogram (CAT-like) assay (Hemker et al. 2002. Pathophysiol. Haemost. Thromb. 32, pp. 249-253; Mann et al. 2007. J. Thromb Haemost. 5, pp. 2055-2061, each incorporated by reference herein in its entirety for all purposes) was evaluated. For the CAT-like assays, multidonor human citrate plasma from healthy individuals, human FVII-deficient plasma and normal rabbit citrate plasma were used. Thrombin (also referred to as Factor IIa, or activated blood coagulation factor II) generation was initiated either with human relipidated TF (in human plasma) or with rabbit relipidated TF (in rabbit plasma).

hI-con1 was maintained frozen at −70° C. until use. Each sample included 3.0 mg hI-con1/mL in formulation buffer (15 mM HEPES, 150 mM NaCl, 5 mM CaCl₂, 25 mM Arginine, 0.01% Tween 80, pH 7.4).

Human plasma FVIIa in 50% glycerol was purchased from Haematologic Technologies, Inc., 57 River Road, Essex Junction, Vt. 05452. It was stored at −20° C. until use. Before use, it was diluted to 10 nM in the formulation buffer (15 mM HEPES, 150 mM NaCl, 5 mM CaCl₂, 25 mM Arginine, 0.01% Tween 80, pH 7.4).

Spectrozyme FXa (#222), lipidated recombinant human TF reagent (Catalog #4500L) and lipidated recombinant rabbit TF were purchased from American Diagnostica, Inc. (Stamford, Conn.), pooled normal human plasma (Lot #IR 11-020711) and rabbit plasma (Lot #26731) were purchased from Innovative Research Novi, Mich. 48377), congenital FVII-deficient plasma (Catalog #0700) was purchased from George King Bio-Medical, Inc. (Overland Park, Kans.) and human factor X (hFX) (#HCX-0050) and Phe-Pro-Arg-chloromethylketone (FPRck; Catalog #FPRCK-01), corn trypsin inhibitor (CTI; Catalog #CTI-01) were purchased from Haematologic Technologies, Inc (Essex Junction, Vt., USA). Fluorogenic substrate Z-Gly-Gly-Arg-AMC.HCl was purchased from Bachem (Torrance, Calif.) and ethylenediaminetetraacetic acid disodium salt dihydrate (EDTA; #E5134), NaCl (#S7653) and HEPES (#H3375) were purchased from Sigma (St. Louis, Mo.). HBS buffer, pH 7.4 contained 150 mM NaCl, 2 mM CaCh and 20 mM HEPES.

Active site inhibited FVIIa (FVIIai) was produced in house. 1, 2-Dioleolyl-sn-Glycero-3-Phospho-L-Serine (PS; #840035) and 1, 2-DioleoyJ-sn-Glycero-3-Phosphocholine (PC; #850375) were purchased from Avanti Polar Lipids, Inc. (Alabaster, Ala., USA). Phospholipid vesicles (PCPS) composed of 25% PS and 75% PC were prepared as described in Higgins and Mann 1983, incorporated by reference herein in its entirety for all purposes.

### Extrinsic FXase

Lipidated recombinant human TF (0.1 nM) was incubated with either 5 nM plasma FVIIa or 5 nM hI-con1 or mixture of both (each at 5 nM) and 100 μM PCPS for 10 min at 37° C. FX (4 μM) was added and at selected time points (0-5 min.) 10 μL aliquots of the reaction mixture were quenched into 170 μL HBS-0.1% PEG-20 mM EDTA. Twenty μL of Spectrozyme FXa (0.2 mM) was added and the rate of substrate hydrolysis was measured as an increase in absorbance at 405 nm (mOD/min).

### Thrombin Generation (CAT-Like) Assay

Corn trypsin inhibitor (CTI) at a final 0.1 mg/mL concentration was added to citrate plasma and 80 μL of this plasma was transferred into Immulon® 96-well plate (Thermo Electron Co., Waltham Mass.). When desired, hI-con1, plasma FVIIa and FVIIai were added at selected concentrations. Twenty μL of 5 pM TF and 20 μM PCPS mixture (both concentrations final) were added to CTI-plasma and incubated for 3 min. Thrombin generation was initiated by the addition of 20 μL of 2.5 mM ZGly-Gly-ArgAMC.HCl in HBS containing 0.1 M CaCl₂. Final concentration of substrate was 416 μM and that of CaCl₂) was 15 mM Thrombin generation curves were generated using Thrombinoscope BY software.

### Results

Comparison of hI-con1 with Plasma FVIIa in the Extrinsic FXase

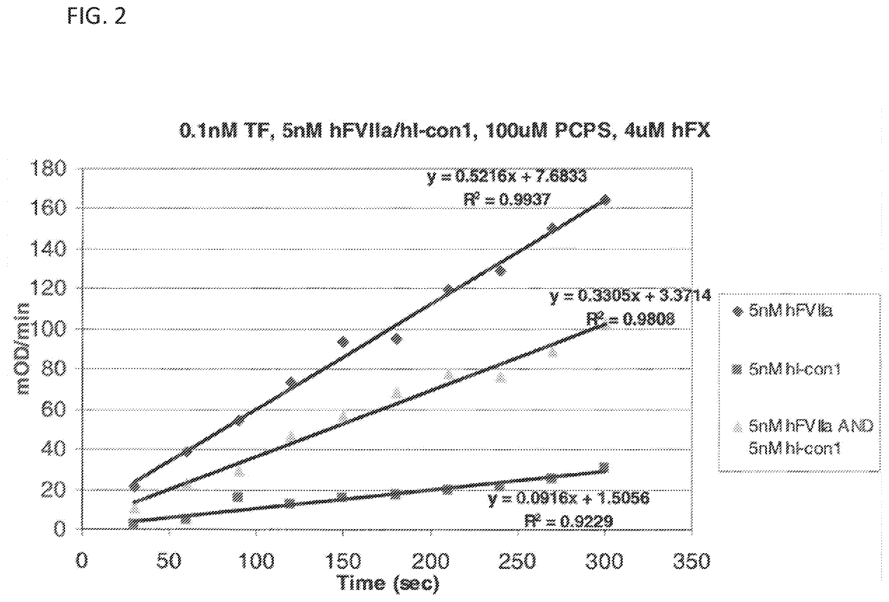

FXa-generating efficiency of two forms of FVIIa and of their mixture was determined in a chromogenic assay. hI-con1 was less active than plasma FVIIa. Activity of hI-con1 was 18% of that observed for plasma FVIIa. When both proteins were added at equimolar (5 nM) concentration, the rate of FXa generation in the middle between the rates observed for individual proteins, indicating that hI-con1 competes with plasma FVIIa for the limited amount of TF (FIG. 2). These data also suggest that hI-con1 has similar affinity for TF as plasma FVIIa.

### Thrombin Generation in Normal Human Plasma: The Effect of FVIIai

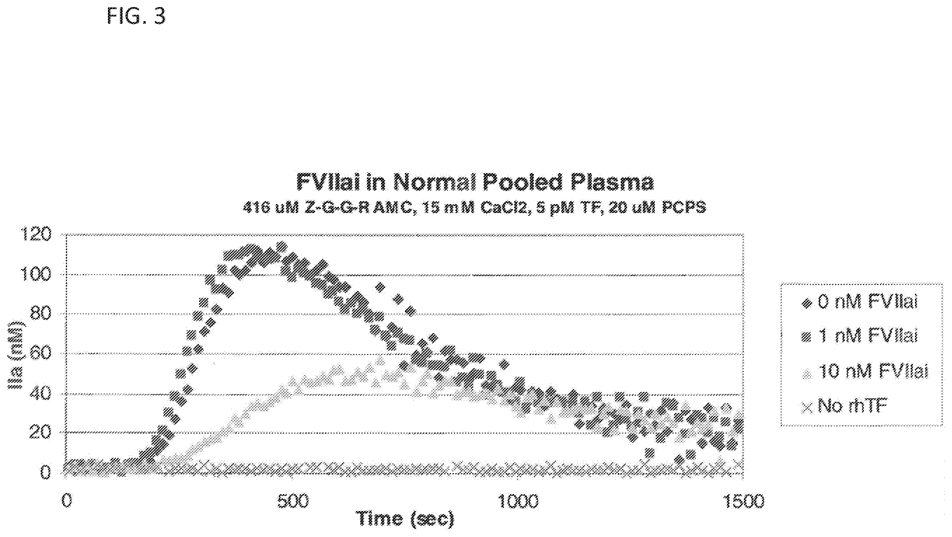

It was hypothesized that due to the low activity of the hI-con1 tissue factor (TF) complex in the extrinsic FXase, hI-con1 could act as an inhibitor by binding TF into an inefficient complex and preventing formation of an efficient complex between plasma FVIIa and TF. To test this hypothesis, the effect of a known inhibitor of coagulation, i.e., active site inhibited FVIIa (Kjalke et al. 1997), on thrombin generation in normal human plasma was evaluated. FVIIai at 1 nM concentration had no effect on thrombin generation initiated with lipidated human TF (FIG. 3). However, at 10 nM, FVIIai prolonged the lag phase of thrombin generation and significantly suppressed both the maximum rate of thrombin generation and the maximum levels of thrombin produced. No thrombin generation was observed in the absence of TF.

Thrombin Generation in Normal Human Plasma: The Effect of hI-con1

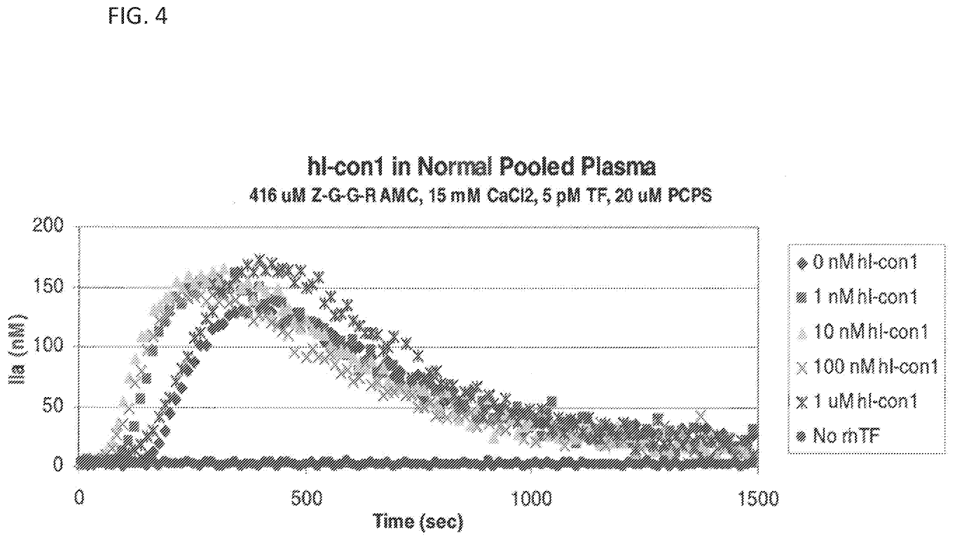

hI-con1 was titrated into normal human plasma initiated with TF to generate thrombin. Varying concentrations of hI-con1 was used, however even at extremely high hI-con1 concentrations (1 μM), no inhibition of thrombin generation was observed (FIG. 4).

### Thrombin Generation in Congenital FVII-Deficient Human Plasma

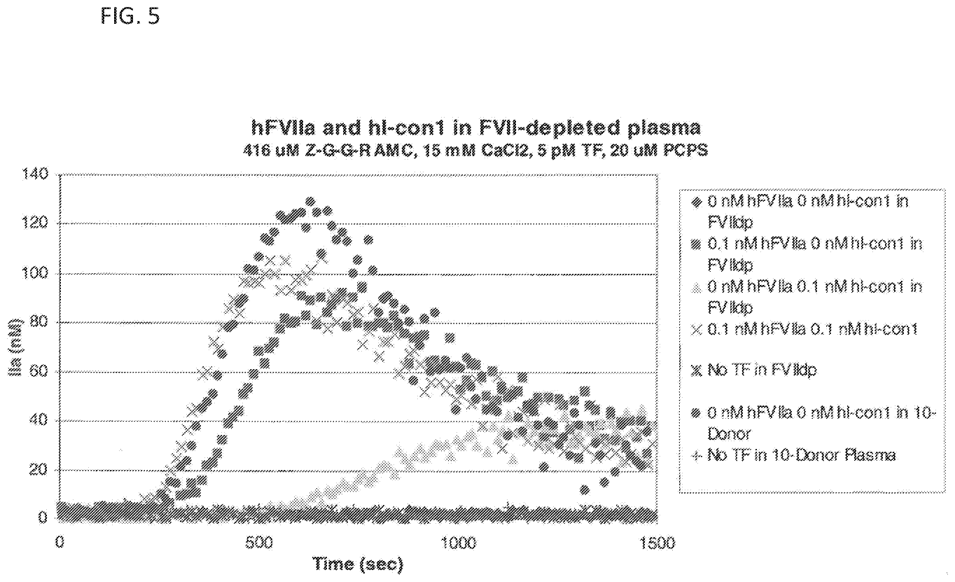

No thrombin generation was observed upon the addition of lipidated human TF to congenital FVII-deficient plasma, indicating that there no detectable functional FVIIa in that plasma (FIG. 5). An addition of 0.1 nM plasma FVIIa together with TF produced thrombin generation profile slightly lower than that observed in normal human plasma. An addition of 0.1 nM hI-con1 alone in the presence of TF led to the initiation of thrombin generation, however the process was significantly delayed and suppressed (FIG. 5). This result was consistent with the observation of low hI-con1 activity in the extrinsic FXase. The addition of both plasma FVIIa and hI-con1 at equimolar concentrations (0.1 nM) did not impair thrombin generation initiated with plasma FVIIa alone.

### Thrombin Generation in Normal Rabbit Plasma

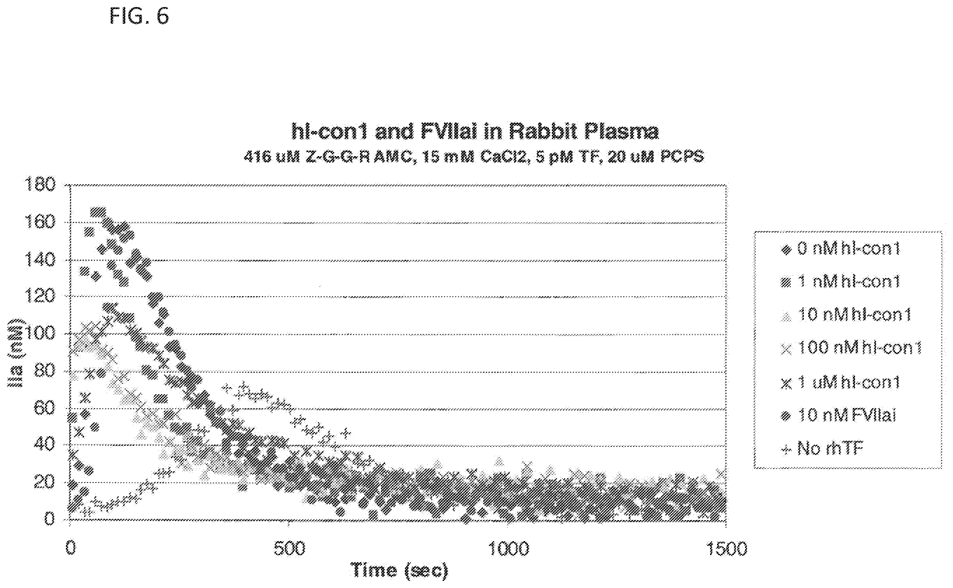

Thrombin generation in rabbit plasma was initiated with lipidated rabbit TF. The addition of 1 nM hI-con1 to this plasma had no pronounced effect on thrombin generation (FIG. 6). Similarly, no pronounced effect was observed when 10 nM FVIIai was added. At higher hI-con1 concentrations (10-1000 nM) some suppression in thrombin generation was observed. However, the control experiment with no TF added led to thrombin generation, suggesting an endogenous presence of TF.

### Thrombin Generation in Centrifuged Rabbit Plasma

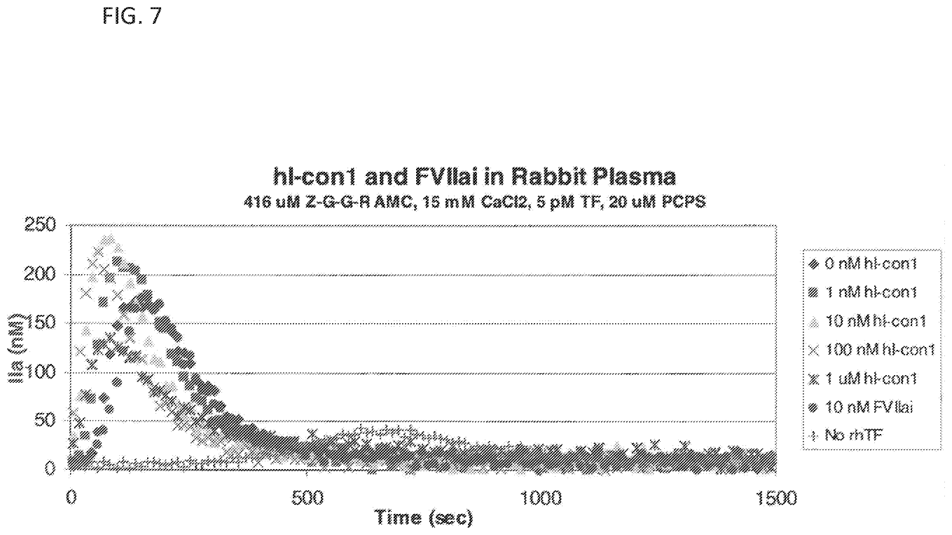

After centrifugation of rabbit plasma, an endogenous thrombin generating activity did not disappear completely, but was significantly decreased (FIG. 7). No suppression in TF-triggered thrombin generation was observed when 10 nM FVIIai was added. Similarly, no suppression was observed when 1-100 nM hI-con1 was added and only a limited decrease in thrombin generation was observed when high concentration (1 μM) hI-con1 was added (FIG. 7). These data indicate that at physiologically-relevant concentrations hI-con1 does not compete for rabbit TF with rabbit FVIIa.

### CONCLUSIONS

hI-con1 does not compete with plasma FVIIa for TF in the citrate plasma environment. hI-con1 has no pronounced (if any) effect on thrombin generation either in human plasma initiated with human TF or in rabbit plasma initiated with rabbit TF. It is not likely that hI-con1 would cause bleeding or thrombotic complications.

### Example 2—Effects of Treatment with hI-con1 on Choroidal Neovascularization in the Pig

In this study, hI-con1 activity in a porcine wet AMD model (Kiilgaard et al., 2005. Acta. Ophthalmol. Scand. 83, pp. 697-704, incorporated by reference herein in its entirety for all purposes) and the optimal dose for the activity was examined. Additionally, the safety of hI-con1 when administered by intravitreal injection was determined.

In this study intravitreal injection of hI-con1 was demonstrated to result in the destruction of established laser-induced CNV in this porcine model. The injections of hI-con1 were well tolerated and the effects were dose-related, with and ED₅₀ of 13.5 μg/dose. A major breakdown product of hI-con1 (100 kDa) was tested and was also well-tolerated and effective with an ED₅₀ of 16.2 μg/dose.

### Test Articles

hI-con1

hI-con1 was provided by Laureate Pharma Inc., 201 E. College Ave, Princeton, N.J., 08540. hI-con1 was maintained frozen at −70° C. until use: Lot PURIC1 080402 (SEC Fr 10-14), two vials each containing 200 μL at 2.0 mg/mL, 1.0 mg/mL, 0.5 mg/mL and 0.25 mg/mL in formulation buffer (15 mM HEPES, 150 mM NaCl, 5 mM CaCl₂), 25 mM Arginine, 0.01% Tween 80, pH 7.4).

100 kD Fragment of hI-con1

The following samples of the 100 kD fragment of hI-con1 were provided by Laureate Pharma Inc. 201 E. College Ave, Princeton, N.J., 08540. The fragment was maintained frozen at −70° C. until use: Lot PURIC1 080402 (SEC Fr 15), two vials each containing 200 μL at 2.0 mg/mL, 1.0 mg/mL, 0.5 mg/mL and 0.25 mg/mL in formulation buffer (15 mM HEPES, 150 mM NaCl, 5 mM CaCl₂), 25 mM Arginine, 0.01% Tween 80, pH 7.4).

### Control Article

The formulation buffer (15 mM HEPES, 150 mM NaCl, 5 mM CaCl₂, 25 mM Arginine, 0.01% Tween 80, pH 7.4) was used as the vehicle control.

### Test Animals

Two studies were conducted, each with groups of five (one group per test article) Yucatan miniature pigs (Sus scrofa), 10-12 weeks old, each weighting approximately 20 kilograms were bought from Professional Veterinary Research (Brownstown, Ind., USA).

### Husbandry

Each pig was maintained in a separate cage within a communal environment that housed four pigs. The lighting was computer controlled and set for a 6 am to 6 pm cycle. The temperature average was 70-72° F., with a variation of +/−1 degree. Humidity was kept between 30 and 70%, with average humidity equal to 33%. The animals were evaluated by the large animal husbandry supervisor and a licensed veterinary technician on arrival and a licensed veterinary technician once weekly until they were euthanized. A veterinarian evaluated the animals to determine if there were any abnormalities or concerns. The animals were quarantined for about 1 week prior to experiments.

### Feed and Water

Daily feed and water were provided to the miniature pigs. They were bedded on hay that served as a feed supplement. The feed was Purina #5084, Laboratory Porcine Grower Diet, Manufactured by Purina Mills, LLC, 555 Maryville University Drive, Suite 500, St. Louis, Mo. 63141, and fed at 2% body weight per day. The water was 0.5 micron filtered tap water. It was not routinely analyzed for contaminants except by the water company and reports are reviewed annually.

### Justification of Species

hI-con1 has limited cross-species activity and the pig is one of the few laboratory animal species in which it is active. The vitreous cavity of the pig is approximately 3 mL, allowing intravitreal injection of reasonable volumes of test article. The pig eye has retinal vascular similarities to humans in addition to several cone-dominant regions of the retina that are similar to the human macula.

### Methods

### Laser-Induced Choroidal Neovascularization

Under general anesthesia, the pupils of the animals were dilated with 1% tropicamide and 2.5% phenylephrine. An indirect ophthalmoscope with a double-frequency YAG laser (532 nm) was used to deliver 74 spots per eye using a 2.2 D lens and the following laser parameters: laser power 1000-1500 mW, duration 0.1 seconds, and repetition rate 500 msec. The laser treatment was designed to yield a microrupture of the Bruch's membrane, generating CNV at 60-70% of the laser spots within two weeks (Bora et al., 2003, incorporated by reference herein in its entirety for all purposes).

### Study Design

The study design is summarized in Table 3 below.

TABLE 3
Study design.

Dose injected (μg)
in each eye

| | | hI-con1 100 kD |
|---|---|---|
| Pig Number | hI-con1 | fragment |
| 1 | 0 | 0 |
| 2 | 25 | 25 |
| 3 | 50 | 50 |
| 4 | 100 | 100 |
| 5 | 200 | 200 |

Choroidal neovascularization was induced on Day 0 in both eyes of two groups of 5 pigs. On Day 10, 100 μL of solutions of hI-con1 (Study 1) or its 100 kD fragment (Study 2) at 0.25, 0.5, 1.0 or 2.0 mg/mL were administered by intravitreal injection into both eyes of the pigs as shown in Table 3. On Day 10, 100 μL of formulation buffer was administered by intravitreal injection into both eyes of the control pigs.

### Test and Control Articles Administration

The animals were anesthetized with a mixture of ketamine hydrochloride (40 mg/kg) and xylazine hydrochloride (10 mg/kg). Injections were administered using a strict sterile technique, which involved scrubbing the lids with a 5% povidone-iodine solution and covering the field with a sterile eye drape. A sterile lid speculum was used to maintain exposure of the injection site. All injections were performed 2 mm from the limbus through the pars plana, using a 30-gauge needle on a 1 mL tuberculin syringe. After injection, a drop of 2% cyclopentolate and antibiotic ointment was placed in the eye. The animals were examined daily for signs of conjunctival injection, increased intraocular pressure, anterior uveitis vitritis, or endophthalmitis, and were sacrificed on Day 14.

### Terminal Procedures

On Day 14, the pigs were anesthetized with an 8:1 mixture of ketamine and xylazine and perfused through the ear vein with 10 mL PBS containing 3 mg/mL fluorescein-labeled dextran with an average molecular weight 2×10⁶ (Sigma, St. Louis, Mo., USA). The eyes were enucleated and four stab incisions were made at the pars plana followed by fixation in 4% paraformaldehyde for 12 hours at 4° C. The cornea and the lens were removed, and the neurosensory retina was dissected from the eyecup and four radial cuts were made from the edge of the eyecup to the equator. The choroid-retinal pigment epithelium (RPE) complex was separated from the sclera and flatmounted on a glass slide in Aquamount with the inner surface (RPE) facing upwards. Flat mounts were stained with a monoclonal antibody against elastin (Sigma) and a Cy3-conjugated secondary antibody (Sigma) and examined with a confocal microscope (Zeiss LSM510, Thornwood, N.Y., USA). The vasculature, filled with dextranconjugated fluorescein, stained green and the elastin in the Bruch's membrane stained red. The level of the Bruch's membrane was determined by confocal microscopy using the intense red signal within a series of z-stack images collected at and around the laser spot. The presence of CNV was indicated by the branching linear green signals above the plane of Bruch's membrane. Absence of CNV was defined under very stringent criteria as the total absence of green fluorescence in the vessels in the spot (see Tezel et al., 2007. Ocular Immunol Inflamm 15, pp. 3-10.

### Statistical Analysis

The percentage of laser spots with CNV at different doses of hI-con1 or its 100 kD fragment was compared pair wise by a chi-square test. The results were plotted against the hI-con1 dose to derive the best-fit curve, which was used to calculate the dose of hI-con1 that reduces the fraction of laser spots with CNV by 50% (ED50). A confidence level of p<0.05 was considered to be statistically significant.

### Results

Effects of Intravitreal Treatment with hI-con1 on CNV

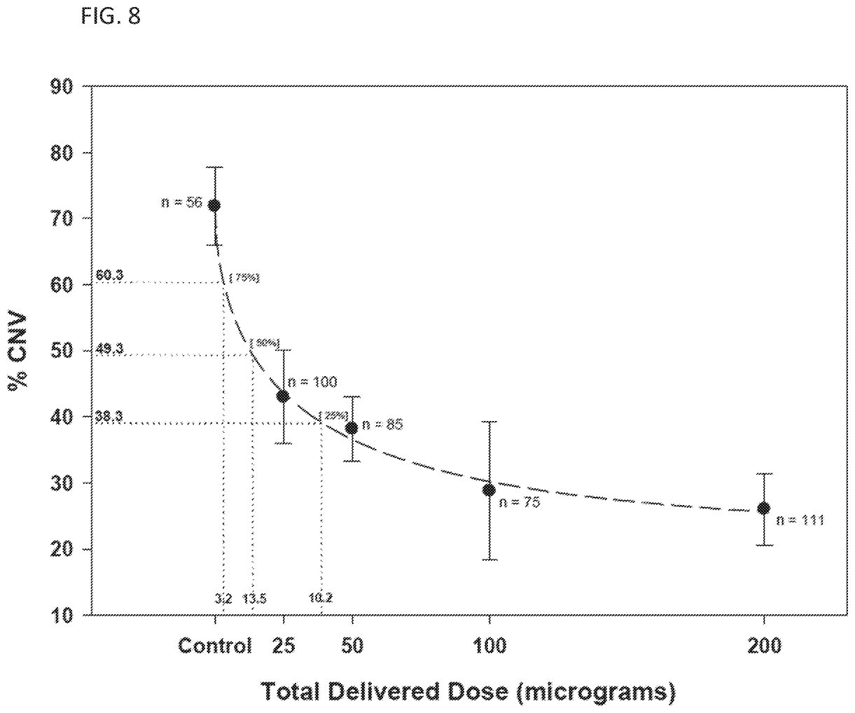

Choroidal neovascularization developed in 71.9±5.8% of the laser spots in control eyes. A single intravitreal injection of hI-con1 on Day 10 in pig eyes (n=2 at each dose) significantly reduced subretinal CNV on Day 14 at all doses tested, i.e., 25-200 μg, Table 4; FIG. 8). The inhibitory effect of hI-con1 fit well to a 5-parameter Sigmoidal Weibull curve. The dose causing a 50% decrease in the yield of CNV (ED50) was 13.5 μg.

TABLE 4
Effects of intravitreal treatment with hI-con1 on CNV incidence
in laser-induced CNV pig model.

| | | | | % spots with | |
|---|---|---|---|---|---|
| | Laser Spots | | | CNV | |
| Pig | Examined | | hI-con1 dose | (Average ± | P value (vs. |
| Number | Left | Right | (μg) | SD) | control) |
| 1 | 25 | 31 | 0 | 71.9 ± 5.8 | |
| 2 | 50 | 50 | 25 | 43.0 ± 7.1 | 0.001 |
| 3 | 36 | 49 | 50 | 38.2 ± 4.9 | <0.001 |
| 4 | 47 | 28 | 100 | 28.8 ± 10.4 | <0.001 |
| 5 | 54 | 57 | 200 | 26.0 ± 5.4 | <0.001 |

Effects of Intravitreal Treatment with 100 kD fragment of hI-con1 on CNV

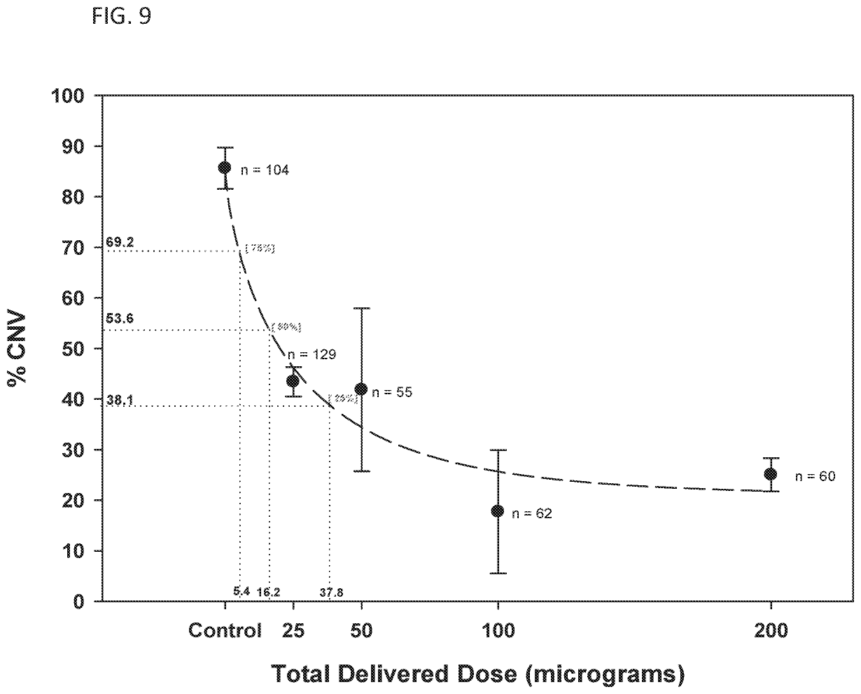

Choroidal neovascularization developed in 85.6±4.1% of the laser spots in control eyes. A single intravitreal injection of the 100 kDa fragment of hI-con1 on Day 10 in pig eyes (n=2 at each dose) significantly reduced subretinal CNV on Day 14 at all doses tested, i.e., 25-200 μg, Table 4, FIG. 9). The inhibitory effect of hI-con1 fit well to a 5-parameter Sigmoidal Weibull curve. The dose causing a 50% decrease in the yield of CNV (ED50) was 16.2 μg.

TABLE 5
Effects of intravitreal treatment with 100 kD fragment of hI-con1
on CNV incidence in laser-induced CNV pig model.

| | | | | % spots with | |
|---|---|---|---|---|---|
| | Laser Spots | | | CNV | |
| Pig | Examined | | hI-con1 dose | (Average ± | P value (vs. |
| Number | Left | Right | (μg) | SD) | control) |
| 1 | 52 | 52 | 0 | 85.6 ± 4.1 | |
| 2 | 73 | 56 | 25 | 43.4 ± 2.9 | <0.001 |
| 3 | 26 | 29 | 50 | 41.8 ± 16.1 | <0.001 |
| 4 | 30 | 32 | 100 | 17.7 ± 12.2 | <0.001 |
| 5 | 14 | 46 | 200 | 25.0 ± 3.3 | <0.001 |

Intravitreal injection of hI-con1 and its 100 kD fragment at doses from 25-200 μg caused significant regression of pre-existing laser-induced CNV 4 days after the injections were administered. The response of the lesions to the injections was clearly dose-related with ED₅₀ doses of 13.5 and 16.2 μg, respectively. These results indicate that the specific activity of the 100 kD fragment of hI-con1 is similar to that of the intact molecule. Doses greater than 100 μg had very little additional decrease in CNV; thus, the efficacious dose in this model is ≤100 μg.

### Example 3—Tissue Cross-Reactivity Study of hI-con1 with Normal Human Tissues

In this study, the binding of hI-con1 to normal human tissues was assessed using standard immunohistochemistry (IHC) techniques, in a standard tissue cross-reactivity (TCR) study. The study was performed utilizing a single batch of biotinylated hI-con1 for IHC staining of normal, as well as positive and negative control human tissues. A positive staining result is indicative of potential toxicities associated with administration of hI-con1 to humans in vivo.

In this model, tissue staining was observed only in the positive control colon carcinoma tumor. All other normal human tissues showed no immunoreactivity. These findings indicate that hI-con1 binding is specific to abnormal tissue, with no binding to normal tissues observed.

### Example 4—Evaluation of the Binding of hI-con1 to Lipidated Tissue Factors

To allow for cross species comparison, a Biacore study of the kinetics of binding of hI-con1 and hFVIIa to human lapidated TF (hTF) and rabbit lipidated TF (rTF) was conducted.

As described in detail below, hI-con1 and hFVIIa both bound with high and approximately equal affinity to lapidated hTF.

### Materials and Methods

Lipidated rabbit tissue factor (rTF; Product #4520L; Lot #051017) purchased from American Diagnostica. Lipidated human tissue factor (hTF; Lot FIL105HO1) supplied by Marin Biological Laboratories, 378 Bel Marin Keys, Novato, Calif. 94949.

hI-con1; 1 ml; 100 μg/ml; MW 157 kDa

Human FVIIa; Lot # A09050525 (Fitzgerald); 1.01 mg/ml; 40 μL/vial; MW 50 kDa

Equipment: Biacore 3000; CM5 Sensor chip

The GE procedure for proteoliposome immobilization (amine coupling) protocol was used to coat PS/PC/rTF on flow cell 2, and PS/PC/hTF on flow cell 3. Flow cells were equilibrated with running buffer (15 mM Hepes, 150 mM NaCl, 5 mM CaCl₂, 25 mM Arginine, 0.01% Tween 80, pH7.4) at a flow rate of 5 μL/min. Kinetic analyses were performed at 37° C. by flowing consecutively increasing concentrations of each analyte (0-10 nM) in the running buffer (15 mM HEPES, 150 mM NaCl, 5 mM CaCl₂), 25 mM Arginine, 0.01% Tween 80, pH 7.4) over the sensor chip for 5 min followed by a 10 min dissociation period at a flow rate of 30 μL/min in parallel.

Analyte binding to the lipidated TF was determined by subtracting the RU values noted in the reference flow cell 1 from flow cell 2 and 3. Binding of analytes to the TFs was monitored in real time to obtain on (ka) and off (kd) rates. The equilibrium dissociation constant (KD) was calculated from the observed ka and kd.

The chips were regenerated with 3 min pulses of 10 mM EDTA in HEPES buffer (20 mM HEPES, 150 mM NaCl, pH 7.4).

Capture of rTF to the chip—Flow cell 2 was coated with rabbit TF (>Resonance Units [RU] 10,000) by amine coupling. Flow cell 3 was coated with human TF (>RU 8,000) by amine coupling.

### Determination of the Amount of Test Ligands (RL) to be Captured on the Chip

In this experiment, the desired level of R_{Max} for the measurement of ligand-analyte interaction was based on the value determined by a previous experiment where rTF captured at 10,000 Resonance Units (“RU”) gave binding of hI-con1 with R_{Max} of 15 RU and hTF captured at 8,000 RU gave binding of hI-con1 with R_{Max} of 10 RU. The amount of the analyte to be captured on the chip depended on the molecular weights of the interacting proteins.

It is determined by the following formula:

R_{Max}=MW_{A}/MW_{L}·R_{L}

MW_{A} is the molecular weight of the analyte (157 kDa for hI-con1, 50 kDa for hFVIIa, and 150 kDa for IgG1).

MW_{L} is the molecular weight of the ligand, in this assay it is expected to be very large (multiple of 35 kDa).

### Flow Rate of the Antibody Solution

The flow rate used for capturing the ligand was 10 μL/min.

For kinetics analysis, the flow rate of 30 μL/min. was used.

### Kinetic Analysis

Based on the saturation concentration of the analyte, binding analysis was performed using saturating analyte concentrations of 0-500 nM for rabbit TF and 0-50 nM for human TF. Chi squared (χ²) analysis was carried out between the actual sensorgram and the calculated on- and off-rates to determine the accuracy of the analysis.

χ² value up to 2 is considered significant (accurate) and below 1 is highly significant (highly accurate).

### Results

The Biacore assay results are provided in Table 6 below.

TABLE 6
Biacore assay results

| | | | | | | | | | Chi |
|---|---|---|---|---|---|---|---|---|---|
| | | Ka | Dk | Rmax | Conc. of | | KA | K_{D} | squared |
| Ligand | Analyte | (1/Ms) | (1/s) | (RU) | analyte | | (1/M) | (M) | (χ²) |
| Rabbit TF | hI-con1 | 5.6 × 10⁴ | 3.0 × 10⁻³ | 4.92 | 0-500 | nM | 1.9 × 10⁷ | 5.3 × 10⁻⁸ | 5.6 × 104 |
| Rabbit TF | hFVIIa | 3.4 × 10⁴ | 1.4 × 10⁻³ | 4.9 | 0-500 | nM | 2.5 × 10⁷ | 4.0 × 10⁻⁸ | 0.06 |
| Human TF | hI-con1 | 6.2 × 10⁵ | 5.5 × 10⁻⁴ | 71.5 | 0-50 | nM | 1.1 × 10⁹ | 8.9 × 10⁻¹⁰ | 3.34 |
| Human TF | hfVIIa | 5.1 × 10⁵ | 9.3 × 10⁻⁴ | 47 | 0-2.0 | nM | 5.5 × 10⁸ | 1.8 × 10⁻⁹ | 0.13 |

As shown in Table 7 below, hI-con1 and hFVIIa both bound with high, and approximately equal, affinity to lipidated hTF. Both ligands also bound to lipidated rTF with approximately 10-fold lower affinities.

TABLE 7
Relative affinities for the binding of hI-con1 and hFVIIa to
hTF and rTF

Dissociation Constant (K_{D})
Analyte

| Ligand | hI-con1 | hFVIIa |
|---|---|---|
| Human Tissue Factor | 8.92 × 10⁻¹⁰M | 1.81 × 10⁻⁹M |
| Rabbit Tissue Factor | 5.32 × 10⁻⁸M | 3.95 × 10⁻⁸M |

### Example 5—Randomized, Double-Masked, Multicenter, Active-Controlled Study Evaluating ICON-1 in Patients with CNV Secondary to Age-Related Macular Degeneration

In this study, the safety and efficacy of intravitreal injections of ICON-1, administered as monotherapy or in combination with ranibizumab (LUCENTIS) compared to ranibizumab (RZB) monotherapy in patients with choroidal neovascularization (CNV) secondary to age-related macular degeneration (AMD) was assessed.

Additionally, the biological activity and pharmacodynamics effect of ICON-1, administered as monotherapy or in combination with ranibizumab (LUCENTIS) compared to ranibizumab monotherapy was assessed.

The study presented in this example is a randomized, double-masked, multicenter, active-controlled study. Patients enrolled in this study were naïve to treatment for CNV. Patients were randomly assigned to one of the following three treatment arms in the selected study eye in a 1:1:1 ratio:
ICON-1 monotherapy (0.3 mg)+sham injection
ranibizumab monotherapy (0.5 mg)+sham injection
ICON-1 (0.3 mg)+ranibizumab (0.5 mg) combination therapy

Randomization was stratified by best-corrected visual acuity (BCVA) letter score in the study eye at baseline (≤54 letters versus≥55 letters) and by study site.

Patients received up to two intravitreal injections at each injection visit. In order to maintain the study mask among the treatment arms, a sham injection was employed in patients receiving monotherapy. There were masked and unmasked study personnel. Intravitreal (IVT) injections were administered by unmasked injecting physicians. Masked evaluating physicians or designated masked site staff members performed all study assessments except for post-injection assessments.

Patients were administered intravitreal injections in the study eye once every four weeks at months 0, 1 and 2. As of Month 3 (at Months 3, 4 and 5) patients were retreated according to their assigned treatment arm, based on their individual observed treatment response. The masked investigator used the following retreatment criteria (based on the category of individual patient response) to determine if treatment was required at these visits:
Loss of ≥5 letters of BCVA due to AMD compared to the previous scheduled visit.
Independent of BCVA change, any anatomical evidenced of increased CNV activity (e.g., new or increased fluid and/or leakage, hemorrhage) compared to the previous scheduled visit.
No BCVA change compared to Baseline (Visit 2), but there is anatomical evidence of persistent CNV activity (e.g., same persistent fluid and CST compared to Baseline.

Rescue treatment with 0.5 mg of ranibizumab was administered to the study eye as an add-on therapy at any time during the 6-month treatment and follow-up period if either of the following conditions occurred:
Loss of ≥15 letters of BCVA due to AMD compared to Baseline (Visit 2).
Loss of ≥10 letters from baseline (Visit 2) of BCVA due to AMD that is confirmed at two consecutive visits. Patients with a loss of ≥10 letters compared to baseline are requested to return within 7 days or as soon as possible for additional follow up at an unscheduled visit.

The masked physician made a determination if rescue treatment was needed according to the above criteria.

If rescue treatment was administered to the study eye during a scheduled injection visit, to ensure that the study masking is maintained, the unmasked physician administers rescue treatment and the patient's scheduled study treatment/re-treatment was as follows.
ICON-1 monotherapy arm: ICON-1 (0.3 mg)+rescue therapy (0.5 mg ranibizumab).
ranibizumab monotherapy arm: ranibizumab (0.5 mg)+sham injection.
combination therapy: ICON-1 (0.3 mg)+ranibizumab (0.5 mg).

If rescue treatment was administered to the study eye at an unscheduled visit, the unmasked physician administered rescue treatment as requested.

If rescue treatment was administered to the study eye, the patient continued with the study visit schedule for the next visit in accordance with the protocol and continued receiving study treatment according to the assigned randomization arm.

Safety was evaluated by tracking of adverse events, clinical laboratory tests (serum chemistry, hematology and coagulation), vital signs measurements, abbreviated physical examinations, slit-lamp biomicroscopy, intraocular pressure (IOP) and dilated ophthalmoscopy. Pharmacodynamic and biological activity were measured by means of BCVA by ETDRS visual acuity chart, spectral-domain optical coherence tomography (sdOCT), color fundus photography (CFP), fundus fluorescein angiography (FA), fundus autofluorescence (FAF), contrast sensitivity, and microperimetry. Pharmacokinetic (PK) and immunogenicity was evaluated by means of measuring plasma concentrations of ICON-1 and anti-drug antibodies.

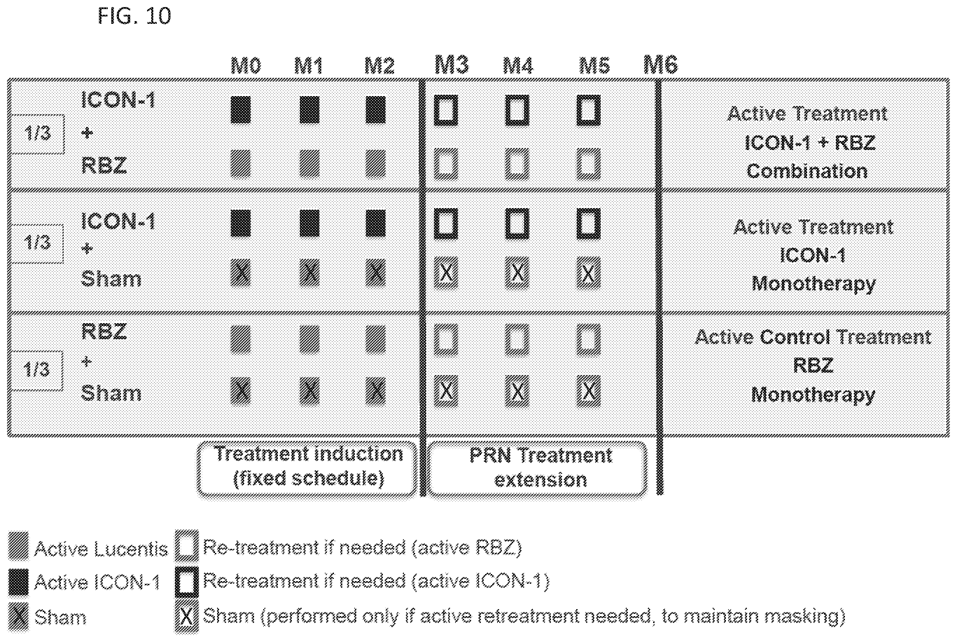

A total of 88 patients were enrolled and randomized in the study: 30 patients each in the ICON-1+RZB combination therapy arm and ICON-1 monotherapy arm, and 28 patients in the RZB monotherapy arm. See FIG. 10 for patient distribution across the three treatment arms.

### Results

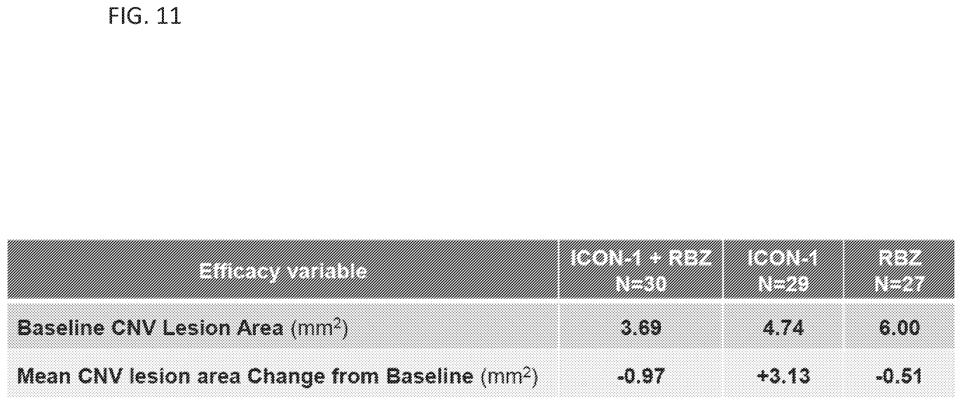

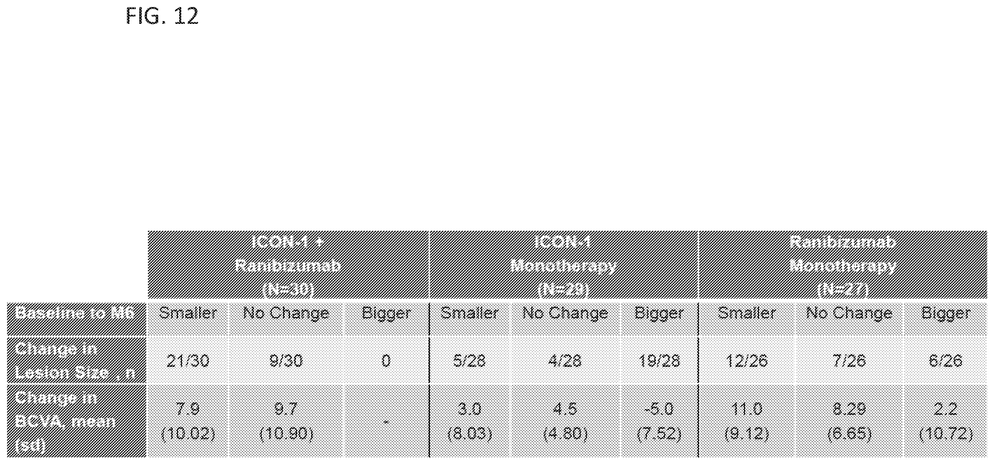

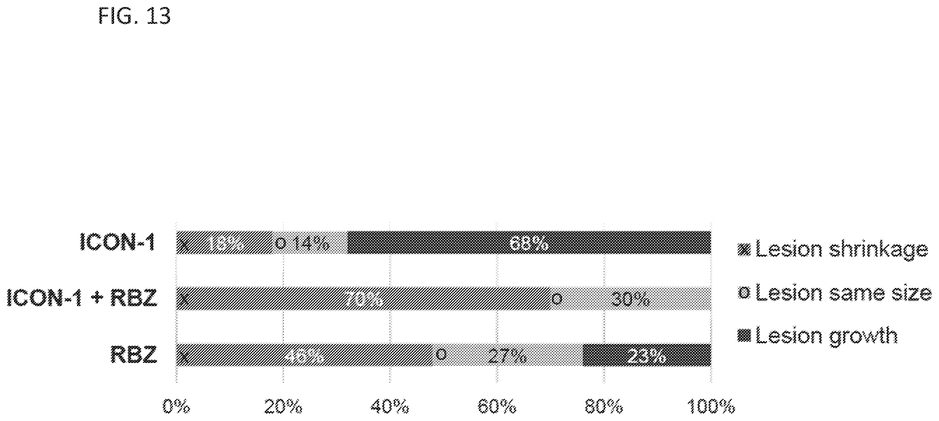

At baseline, the mean total area of CNV lesion was relatively low in all treatment arms. The lowest mean CNV lesion area at baseline was in the ICON-1+RZB combination therapy arm (3.69 mm²), while it was 4.74 mm² for patients in the ICON-1 monotherapy arm and 6.00 mm² for patients in the RZB monotherapy arm. The mean reduction in the CNV lesion area was higher in the ICON-1+RZB combination arm at month 6, with the reduction of more than −0.97 mm² at month 3 that was maintained at month 6. See FIG. 11 and FIG. 12 for tabulated data pertaining to the baseline CNV lesion area and the mean CNV lesion area change from baseline. FIG. 13 provides a visual representation of the proportion of patients in the three treatment arms as they correspond to shrinkage, growth, or no change in lesion size.

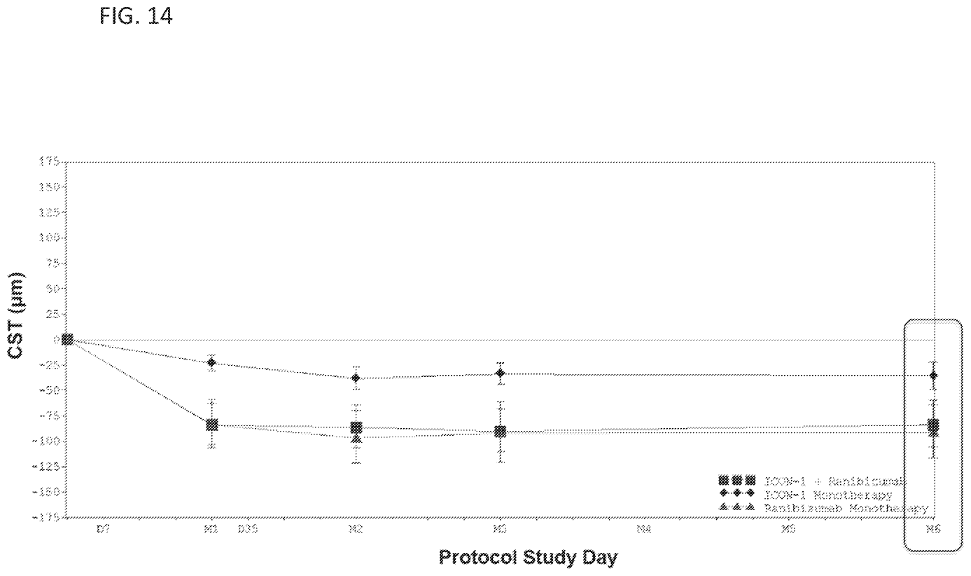

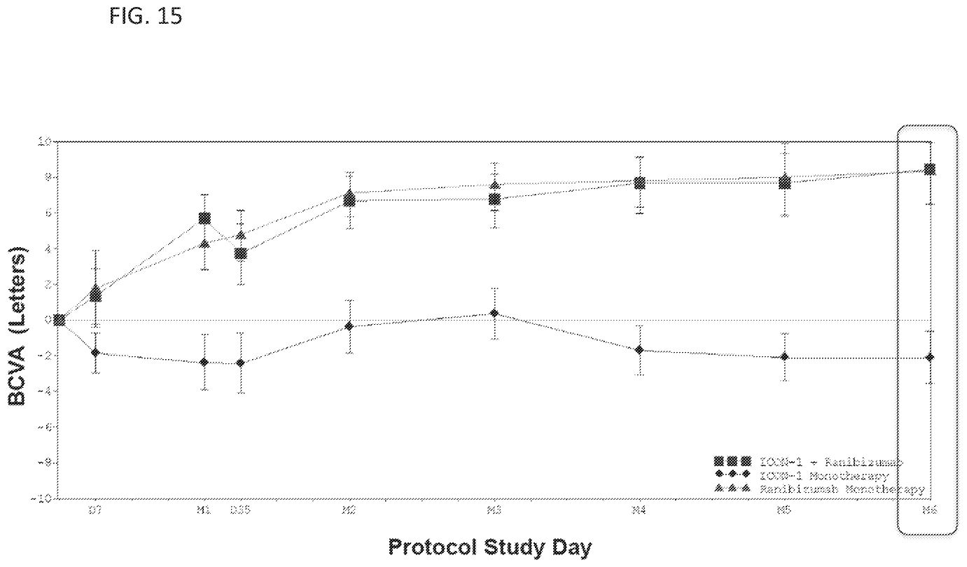

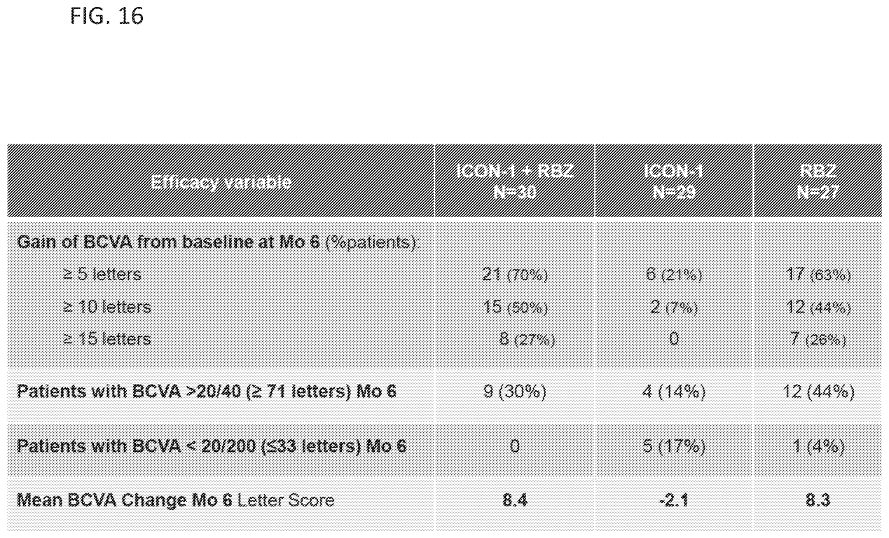

Reduction of the CNV lesion area was greatest in patients receiving ICON-1+RZB combination therapy at month 6. Reduction of CST was observed at month 3 and was maintained from months 3 to 6 in all treatment arms (See FIG. 14). Reduction in CST mirrored and supported the outcomes for BCVA over time as signals of biological activity (See FIG. 15 and FIG. 16).

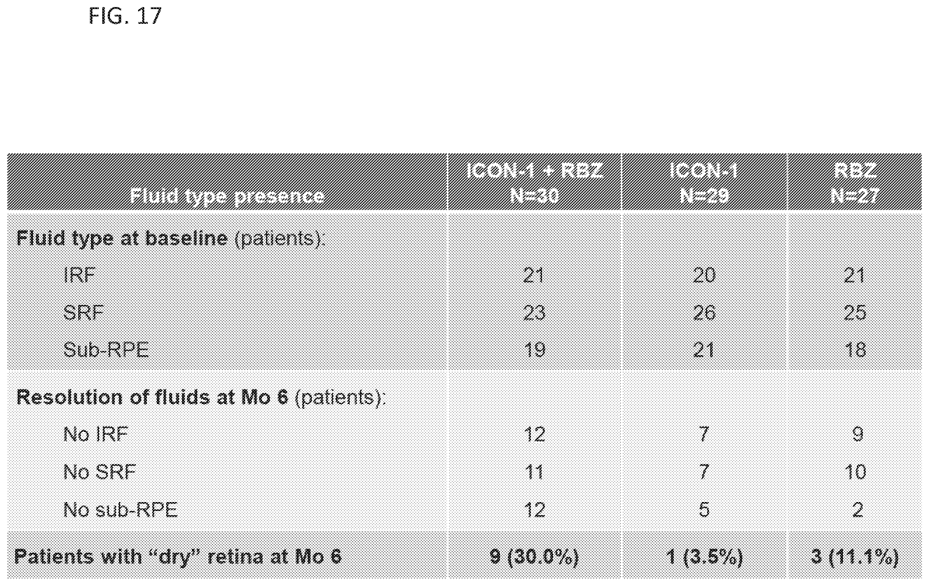

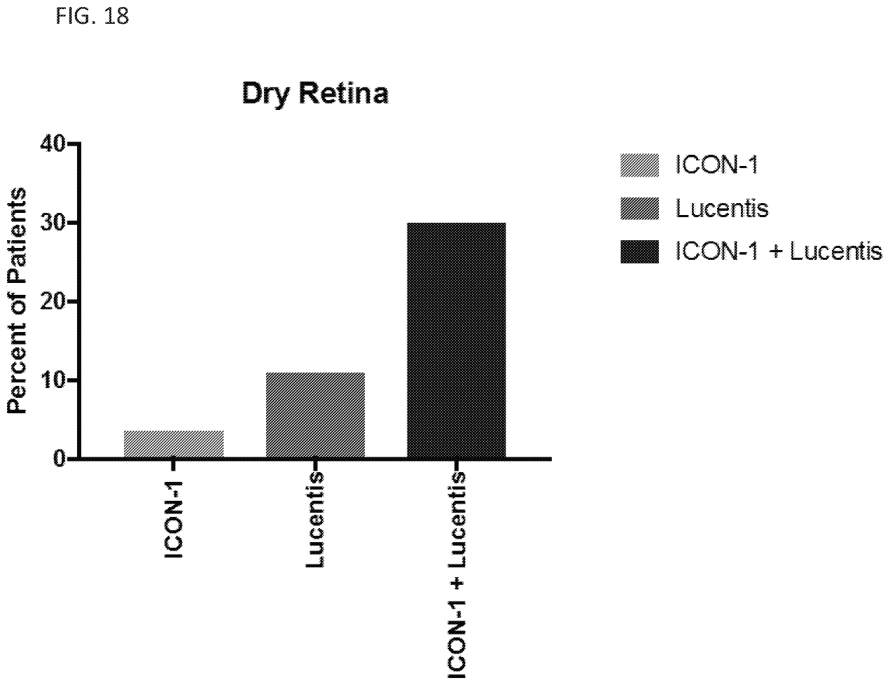

At baseline, all patients across the 3 treatment arms had fluid/exudative presence on the sdOCT, with the presence of subretinal fluid (SRF), intraretinal fluid (IRF) and/or subretinal pigment epithelium fluid (Sub-RPE). At month 6, the absence of any fluids was observed in a higher proportion of patients in the ICON-1+RZB combination therapy arm (30.0%) compared to both the ICON-1 monotherapy (3.4%) and RZB monotherapy (11.1%) arms. In the ICON-1+RZB combination therapy arm, a higher proportion of patients had no fluid present at month 6 (30%) (See FIG. 17 and FIG. 18).

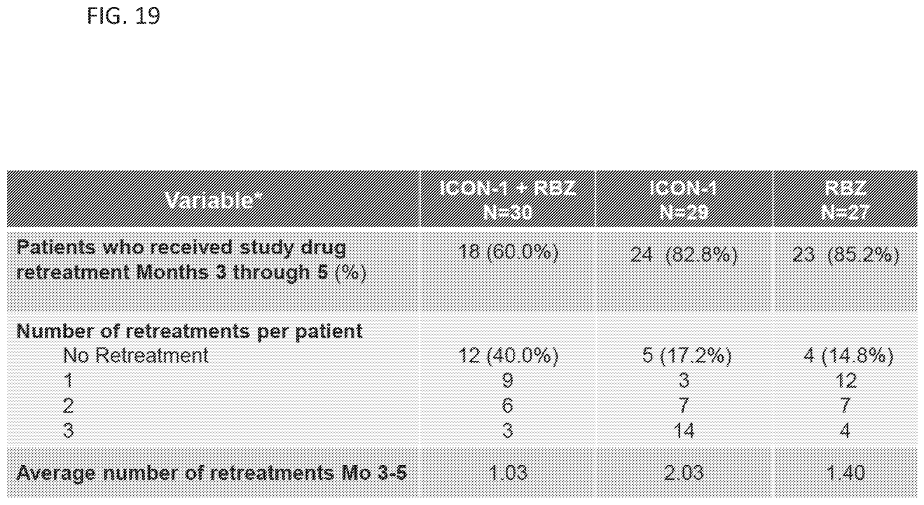

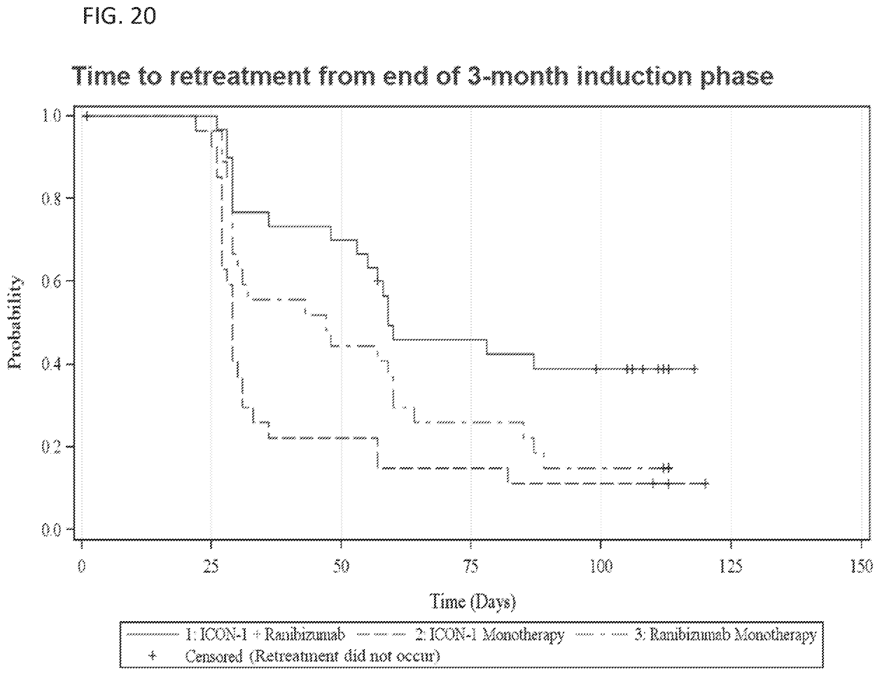

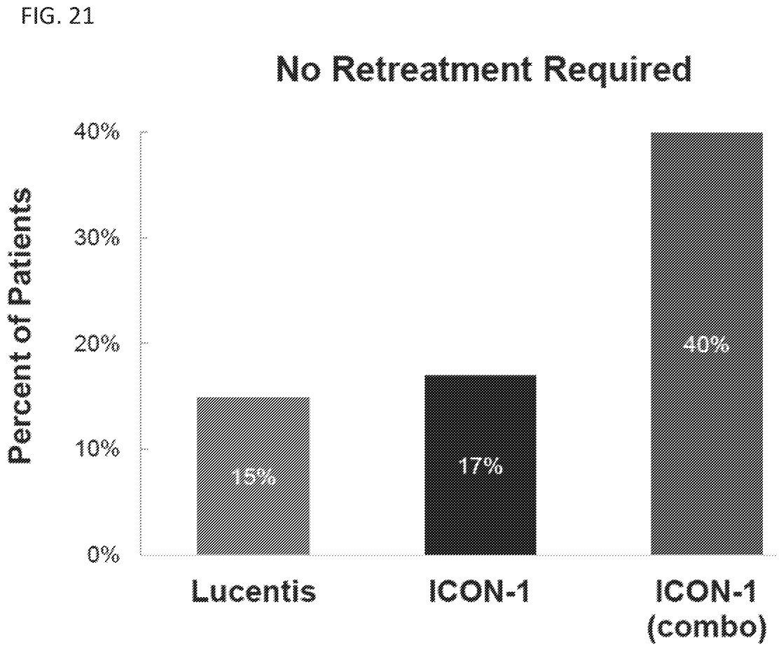

For the patients in the ICON-1+RZB combination therapy arm, there were longer treatment-free intervals from months 3 through 5, and more patients received no retreatment (See FIG. 19). The average number of retreatments per patient was 1.0 in the ICON-1+RZB combination therapy arm, 1.4 in the RZB monotherapy arm and 2.0 in the ICON-1 monotherapy arm. The mean time from treatment end to first retreatment was longer in patients in the ICON-1+RZB combination therapy arm (62.8 days) than in the RZB monotherapy arm (51.7 days) and in the ICON-1 monotherapy arm (38.4 days) (See FIG. 20). More patients receiving ICON-1+RZB combination therapy did not require retreatment (40%) compared with patients receiving RZB monotherapy (14.8%), with a decreased frequency of retreatment and longer treatment-free intervals (See FIG. 21).

The signals of biological activity in both BCVA and CNV lesion changes were achieved, with the lower need for retreatment in patients receiving ICON-1+RZB combination therapy compared to patients receiving RZB monotherapy, suggestive of a synergistic biological effect of CNV modification. In patients receiving ICON-1 monotherapy, the stable BCVA over 6 months and the reduction in exudation and fluid are supporting and suggestive of a biological signal.

### Example 6—Synergism of ICON-1 and Anti-VEGF Antibodies in Patients with Choroidal Neovascularization, Ocular Neovascularization, and Tumor Neovascularization

In this study, the synergistic effect of treatment with hI-con1 and anti-VEGF antibodies is evaluated in patients with CNV, ocular neovascularization, and tumor neovascularization.

The study duration lasts for a period of twelve months, which includes a screening visit to determine in patient meet the study parameters, a baseline randomization visit at month 0, followed by monthly visits from months 1 through 12.

The baseline randomization visit identifies the CNV lesion area and exudation, and measures BCVA.

The study comprises three different groups: a control group that receives anti-VEGF alone, a group that receives doses of anti-VEGF and ICON-1 (0.3 mg), and a group that receives doses of anti-VEGF and ICON-1 (0.6 mg).

Patients in each group receive treatments at months 1, 2, and 3, while months 4-12 are follow up visits in which treatment is administered only if needed, i.e. if CNV activity is observed, based on pre-defined criteria for treatment.

Patients in groups that receive both anti-VEGF and ICON-1 are expected to exhibit an improved best-corrected visual acuity (BCVA) outcome at month 3 and month 12 from the baseline determination, as compared to those receiving only the anti-VEGF treatment. Patients in groups that receive both anti-VEGF and hI-con1 are further expected to exhibit a reduction in mean CNV lesion area at month 3 and month 12 from the baseline determination, as compared to those receiving only the anti-VEGF treatment.

Patients receiving both anti-VEGF and hI-con1 are expected to exhibit at least a decrease in CNV activity such that there is no increase in the area of the lesion and there is no increase in the CNV-associated exudation. Patients receiving both anti-VEGF and hI-con1 are expected to exhibit a reduction of in the area of the lesion and a reduction in CNV-associated exudation. Patients receiving both anti-VEGF and hI-con1 are likely to exhibit regression of the lesion and CNV-associated exudation.

While the study lasts for a period of 12 months, the patients receiving both anti-VEGF and ICON-1 are expected to exhibit a decrease in CNV activity, a reduction of the area of the lesions and a reduction of CNV-associated exudation, and regression of the lesion and CNV-associated exudation earlier in the study than those receiving only anti-VEGF. Furthermore, the durability of treatment in patients receiving both anti-VEGF and hI-con1 is expected to be greater than in patients receiving only anti-VEGF.

### Example 7—Dose-Dependent Response of ICON-1 and Synergism of ICON-1 and VEGF Inhibitor in Laser-Induced CNV

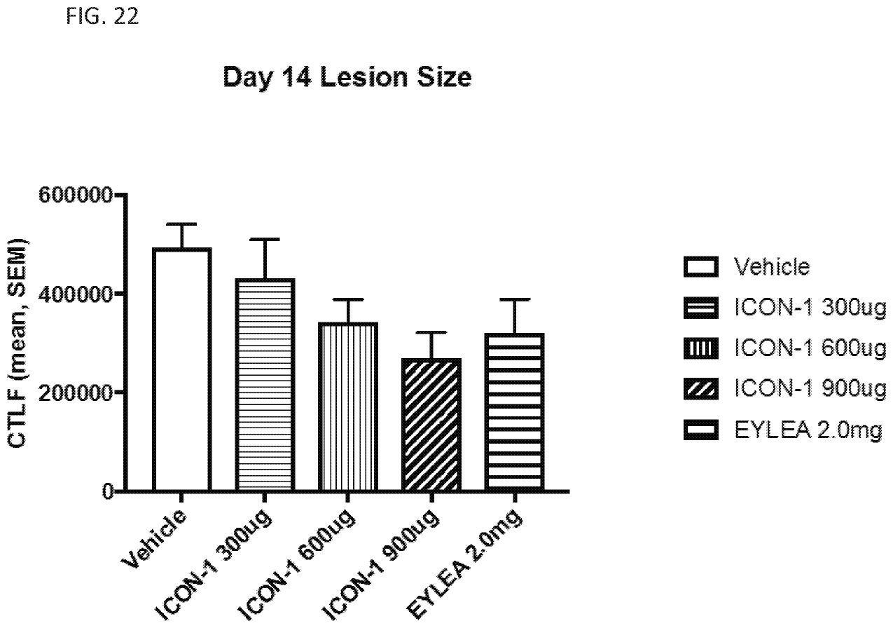

Dose-dependent responses of ICON-1 were determined in ten to twelve week old pigs (Swine/Hampshire Cross) which underwent bilateral laser induction to create ˜6 single laser spots in each eye. Each group consisted of 4 animals, for a total of 48 laser spots per group. For efficacy assessments, ICON-1 at 300 μg, 600 μg, and 900 μg was administered intravitreally on day 7 post-laser treatment. Eylea (aflibercept), a VEGF inhibitor, was administered on day 0 at a dose of 2 mg. Fluorescein Angiography (FA) was used to determine total lesion fluorescence on day 7 as a baseline and day 14 (See FIG. 22). ICON-1 administration was well tolerated with no dose related ocular toxicities and no systemic effects. A dose-dependent reduction in lesion fluorescence, measured by FA, was observed in ICON-1 treated animals at day 14. The largest decrease in lesion fluorescence was the 900 μg group compared to the vehicle-treated group (See FIG. 22). The FA measurements were expressed as Corrected total lesion fluorescence (CTFL); CTFL=integrated density−(area of selected cell×mean fluorescence of background readings).

The Synergism of ICON-1 and a VEGF inhibitor were determined in in ten to twelve week old pigs (Swine/Hampshire Cross) which underwent bilateral laser induction to crease ˜6 single laser spots in each eye. Each group consisted of 4 animals, for a total of 48 laser spots per group. For efficacy assessments, ICON-1 at 600 μg was administered intravitreally on day 7 post-laser treatment. Eylea (aflibercept), a VEGF inhibitor, was administered on day 0 at a dose of 2 mg. Fluorescein Angiography (FA) was used to determine total lesion fluorescence on day 7 as a baseline and day 14 (See FIG. 23). ICON-1 administration was well tolerated with no dose related ocular toxicities and no systemic effects. Greater reduction in lesion fluorescence was observed in the ICON-1+Eylea combination treated animals at day 14, compared to the vehicle treated group and to the ICON-1 along and Eylea (aflibercept) alone groups (See FIG. 23). The FA measurements were expressed as Corrected total lesion fluorescence (CTFL); CTFL=integrated density−(area of selected cell×mean fluorescence of background readings)

### Example 8—Pharmacological Study Evaluating the Efficacy of ICON-1.5 in a Model of Laser-Induced Choroidal Neovascularization (CNV)

This study evaluates the efficacy of intravitreal injections of one-armed FVII-Fc immunoconjugates, administered as monotherapy or in combination with anti-VEGF agents such as ranibizumab (LUCENTIS) or aflibercept (EYLEA) compared to anti-VEGF monotherapy in a rabbit model of laser induced choroidal neovascularization (CNV). Four animals per group, and six groups. The groups consist of (1) vehicle, (2) 300 μg, (3) 600 μg, (4) 900 μg, (5) aflibercept 2.0 mg, and (6) aflibercept 2.0 mg+ICON-1 (ICON-1.5 in parallel study) at 600 μg.

Rabbits are lasered in both eyes (OU) on Day 0 (DO). Test articles and vehicle are dosed bilaterally via intravitreal (IVT) injection on D7. Ranibizumab (LUCENTIS) or aflibercept (EYLEA) are dosed on day 0 immediately after laser (DO). In the combination group (group 7), anti-VEGF agents are injected on DO and one-armed FVII-Fc immunoconjugates on D7.

Ocular Examination: Mydriasis for ocular examination is done using topical 1% tropicamide HCL (one drop in each eye 15 minutes prior to examination). Complete ocular examination (modified Hackett and McDonald) using a slit lamp biomicroscope and indirect ophthalmoscope to evaluate ocular surface morphology, anterior segment and posterior segment inflammation, cataract formation, and retinal changes are conducted by a veterinary ophthalmologist at baseline and D14.

Fluorescein angiography (FA): FA is done in both eyes of all animals on D7, D10, and D14 after laser. Mydriasis for FA is done using topical 1% Tropicamide HCL (one drop in each eye 15 minutes prior to examination). Full FA is performed for 1-5 minutes after intravenous sodium fluorescein injection (12 mg kg⁻¹). A reader analyzes the masked images obtained. The area of maximal fluorescein leakage is measured using Image J for each lesion.

All eyes are collected for in situ hybridization (ISH) and flat-mount analysis of choroidal vascularity detected by fluorescein isothiocyanate-dextran staining.

### Example 9—Pharmacological Study Evaluating the Efficacy of ICON-1.5 in a Swine Model of Laser-Induced Choroidal Neovascularization (CNV)

This study evaluates the efficacy of intravitreal injections of one-armed FVII-Fc immunoconjugates, administered as monotherapy or in combination with anti-VEGF agents such as ranibizumab (LUCENTIS) or aflibercept (EYLEA) compared to anti-VEGF monotherapy in a swine model of laser induced choroidal neovascularization (CNV). Four animals per group, and six groups. The groups consist of (1) vehicle, (2) 300 μg, (3) 600 μg, (4) 900 μg, (5) aflibercept 2.0 mg, and (6) aflibercept 2.0 mg+ICON-1 (ICON-1.5 in parallel study) at 600 μg.

Pigs are lasered in both eyes (OU) on Day 0 (DO). Test articles and vehicle are dosed bilaterally via intravitreal (IVT) injection on D7. Ranibizumab (LUCENTIS) or aflibercept (EYLEA) are dosed on day 0 immediately after laser (DO). In the combination group (group 7), anti-VEGF agents are injected on DO and one-armed FVII-Fc immunoconjugates on D7.

Ocular Examination: Mydriasis for ocular examination is done using topical 1% tropicamide HCL (one drop in each eye 15 minutes prior to examination). Complete ocular examination (modified Hackett and McDonald) using a slit lamp biomicroscope and indirect ophthalmoscope to evaluate ocular surface morphology, anterior segment and posterior segment inflammation, cataract formation, and retinal changes are conducted by a veterinary ophthalmologist at baseline and D14.

Fluorescein angiography (FA): FA is done in both eyes of all animals on D7, D10, and D14 after laser. Mydriasis for FA is done using topical 1% Tropicamide HCL (one drop in each eye 15 minutes prior to examination). Full FA is performed for 1-5 minutes after intravenous sodium fluorescein injection (12 mg kg-1). A reader analyzes the masked images obtained. The area of maximal fluorescein leakage is measured using Image J for each lesion.

All eyes are collected for ISH and flatmount analysis.

While the described invention has been described with reference to the specific embodiments thereof it should be understood by those skilled in the art that various changes may be made and equivalents may be substituted without departing from the true spirit and scope of the invention. In addition, many modifications may be made to adopt a particular situation, material, composition of matter, process, process step or steps, to the objective spirit and scope of the described invention. All such modifications are intended to be within the scope of the claims appended hereto.

Patents, patent applications, patent application publications, journal articles and protocols referenced herein are incorporated by reference in their entireties, for all purposes.

1. A method for preventing, inhibiting, or reversing wet age-related macular degeneration (AMD) in an eye of a patient in need thereof, comprising, administering to the patient in multiple dosing sessions:
(a) an effective amount of an immunoconjugate dimer, wherein the monomer subunits of the dimer each comprises a mutated human factor VIIa (fVIIa) protein conjugated to the human immunoglobulin G1 (IgG1) Fc domain; and
(b) a VEGF inhibitor;
wherein the administration results in an improved best-corrected visual acuity (BCVA) outcome, improved reduction in mean choroidal neovascularization (CNV) lesion area, improved reduction in CNV exudation, or improved durability of treatment compared to patients having been administered the VEGF inhibitor alone.

2. A method for preventing, inhibiting, or reversing ocular neovascularization in an eye of a patient in need thereof, comprising, administering to the patient in multiple dosing sessions, a composition comprising:
(a) an effective amount of an immunoconjugate dimer, wherein the monomer subunits of the dimer each comprises a mutated human factor VIIa (fVIIa) protein conjugated to the human immunoglobulin G1 (IgG1) Fc domain; and
(b) a VEGF inhibitor;
wherein the administration results in an improved best-corrected visual acuity (BCVA) outcome, improved reduction in mean choroidal neovascularization (CNV) lesion area, improved reduction in CNV exudation, or improved durability of treatment compared to patients having been administered the VEGF inhibitor alone.

3. A method for reversing tumor neovascularization in an eye of a patient in need thereof, comprising administering to the patient in multiple dosing sessions a composition comprising:
(a) an effective amount of an immunoconjugate dimer, wherein the monomer subunits of the dimer each comprises a mutated human factor VIIa (fVIIa) protein conjugated to the human immunoglobulin G1 (IgG1) Fc domain; and
(b) a VEGF inhibitor;
wherein the administration results in an improved best-corrected visual acuity (BCVA) outcome, improved reduction in mean choroidal neovascularization (CNV) lesion area, improved reduction in CNV exudation, or improved durability of treatment compared to patients having been administered the VEGF inhibitor alone.

4. The method of any one of claims 1-3 or 65-67, wherein the VEGF inhibitor comprises an anti-VEGF antibody.

5. The method of any one of claims 1-3 or 65-67, wherein the immunoconjugate dimer is a homodimer.

6. The method of any one of claims 1-3 or 65-67, wherein the immunoconjugate dimer is a heterodimer.

7. The method of any one of claims 1-3 or 65-67, wherein the reduction in CNV exudation is a reduction in intraretinal fluid, subretinal fluid, or subretinal pigment epithelium fluid.

8. The method of any one of claims 1-3 or 65-67, wherein the reduction in CNV exudation is a reduction in intraretinal fluid, subretinal fluid, and subretinal pigment epithelium fluid.

9. The method of any one of claims 1-3 or 65-67, wherein at least one of the monomer subunits of the immunoconjugate comprises a mutated human fVIIa domain comprising a single point mutation at Lys341 or Ser 344.

10. The method of claim 9, wherein the single point mutation is to an Ala residue.

11. The method of claim 10, wherein the single point mutation is Lys341 to Ala341.

12. The method of claim 10, wherein the single point mutation is Ser344 to Ala344.

13. The method of any one of claims 2, 3, 66, or 67, wherein the ocular neovascularization is associated with proliferative diabetic retinopathy, wet age-related macular degeneration (AMD), retinopathy of prematurity (ROP), or neovascular glaucoma.

14. The method of any one of claims 2, 3, 66, or 67, wherein the ocular neovascularization is secondary to proliferative diabetic retinopathy, wet age-related macular degeneration (AMD), retinopathy of prematurity (ROP), or neovascular glaucoma.

15. The method of any one of claims 1-3 or 65-67, wherein the ocular neovascularization is choroidal neovascularization.

16. The method of claim 15, wherein the patient has been previously diagnosed with wet age-related macular degeneration (AMD) in the eye.

17. The method of claim 15, wherein the choroidal neovascularization is secondary to wet AMD.

18. The method of claim 16 or 17, wherein the eye of the patient has not been previously treated for choroidal neovascularization or wet AMD.

19. The method of claim 16 or 17, wherein the patient has previously been treated for choroidal vascularization with anti-vascular endothelial growth factor (VEGF) therapy, laser therapy, or surgery.

20. The method of any one of claims 1-3 or 65-67, wherein administering comprises intravitreal injection at each dosing session.

21. The method of any one of claims 1-3 or 65-67, wherein administering comprises suprachoroidal injection at each dosing session.

22. The method of any one of claims 1-3 or 65-67, wherein the multiple dosing sessions comprise two or more, three or more, four or more, five or more, six or more, seven or more, eight or more, nine or more, ten or more, eleven or more, twelve or more, thirteen or more, fourteen or more, fifteen or more, sixteen or more, seventeen or more, eighteen or more, nineteen or more, or twenty or more dosing sessions.

23. The method of any one of claims 1-22, wherein each dosing session is spaced apart by from about 20 days to about 50 days, or from about 20 days to about 40 days, or from about 20 days to about 30 days.

24. The method of claim 23, wherein the multiple dosing sessions comprise 12 to 24 dosing sessions.

25. The method of any one of claims 1-3 or 65-67, wherein administering comprises intravitreal injection into the eye of the patient once every 28 days, once every 30 days, or once every 35 days.

26. The method of any one of claims 1-3 or 65-67, wherein the immunoconjugate comprises the amino acid sequence of SEQ ID NO: 2 or 3.

27. The method of claim 26, wherein the immunoconjugate comprises the amino acid sequence of SEQ ID NO: 2.

28. The method of claim 26, wherein the immunoconjugate comprises the amino acid sequence of SEQ ID NO: 3.

29. The method of claim 26, wherein the immunoconjugate is encoded by a polynucleotide sequence comprising SEQ ID NO:4.

30. The method of claim 26, wherein the immunoconjugate is encoded by a polynucleotide sequence comprising SEQ ID NO: 5.

31. The method of any one of claims 1-3 or 65-67, wherein administering comprises intravenous administration.

32. The method of claim 3, wherein administering comprises intratumoral injection.

33. The method of any one of claims 1-3 or 65-67, wherein the improved reduction in CNV lesion area or CNV exudation is reduced by at least about 5%, at least about 10%, at least about 20%, at least about 30%, at least about 40%, or at least about 50%.

34. The method of any one of claims 1-3 or 65-67, wherein the improved durability of treatment is increased by at least about 5%, at least about 10%, at least about 20%, at least about 30%, at least about 40%, or at least about 50%.

35. The method of any one of claims 1-3 or 65-67, wherein subsequent to the multiple dosing sessions, the retinal thickness of the eye of the patient is reduced in the eye of the patient, as compared to the retinal thickness of the eye of patients having been administered the VEGF inhibitor alone.

36. The method of claim 35, wherein the retinal thickness is reduced by at least about 50 μm, at least about 100 μm, at least about 150 μm, at least about 175 μm, at least about 200 μm, at least about 225 μm, or at least about 250 μm.

37. The method of claim 35, wherein the retinal thickness is reduced by at least about 5%, at least about 10%, at least about 20%, at least about 30%, at least about 40%, or at least about 50%.

38. The method of any one of claims 35-37, wherein the decreased retinal thickness is decreased central retinal subfield thickness (CST), decreased center point thickness (CPT), or decreased central foveal thickness (CFT).

39. The method of any one of claims 1-3 or 65-67, further comprising measuring the intraocular pressure (IOP) in the eye of the patient prior to each dosing session.

40. The method of any one of claims 1-3 or 65-67, further comprising measuring the IOP in the eye of the patient about 20 minutes, about 30 minutes, about 40 minutes, about 50 minutes, or about 1 hour after each dosing session.

41. The method of any one of claims 1-3 or 65-67, comprising measuring the IOP in the eye of the patient about 20 minutes, about 30 minutes, about 40 minutes, about 50 minutes, or about 1 hour prior to each dosing session.

42. The method of any one of claims 39-41, wherein the IOP is measured via tonometry.

43. The method of any one of claims 1-3 or 65-67, wherein the VEGF inhibitor is present in the same composition as the immunoconjugate.

44. The method of any one of claims 1-3 or 65-67, wherein the VEGF inhibitor is present in a different composition than the immunoconjugate.

45. The method of any one of claims 1-3 or 65-67, wherein the VEGF inhibitor is aflibercept.

46. The method of claim 4, wherein the anti-VEGF antibody is ranibizumab.

47. The method of claim 45, wherein the dosage of ranibizumab is from about 0.2 mg to about 1 mg.

48. The method of claim 46 or 47, wherein the dosage of ranibizumab is 0.3 mg, 0.4 mg, 0.5 mg, 0.6 mg, or 0.7 mg.

49. The method of any one of claims 1-48, wherein ranibizumab is administered to the eye of the patient via an intravitreal injection.

50. The method of claim 45, wherein the VEGF inhibitor is administered at a dosage from about 0.2 mg to about 0.7 mg.

51. The method of claim 49, wherein the VEGF inhibitor is administered at a dosage of about 0.6 mg.

52. The method of claim 49, wherein the VEGF inhibitor is administered at a dosage of 0.6 mg.

53. The method of claim 49, wherein the VEGF inhibitor is administered at a dosage of about 0.3 mg.

54. The method of claim 49, wherein the VEGF inhibitor is administered at a dosage of 0.3 mg.

55. The method of any one of claims 1-3 or 65-67, wherein the multiple dosing sessions comprise administration once per month.

56. The method of claim 55, wherein the multiple dosing sessions comprise administration once per month for the first three months, followed by monthly treatments in months 4-12 only when CNV activity is observed.

57. The method of claim 55, wherein the multiple dosing sessions comprise administration once per month for the first three months, followed by monthly treatments in months 4-6 only when CNV activity is observed.

58. The method of any one of claims 1-48, wherein the composition comprising the effective amount of the VEGF inhibitor is administered to the eye of the patient via an intravitreal injection.

59. The method of claim 49, wherein the composition comprising the effective amount of the VEGF inhibitor is administered at each of the multiple dosing sessions.

60. The method of any one of claims 1-3 or 65-67, wherein each dosing session comprises the administration of between about 200 μg and about 600 μg of the immunoconjugate dimer.

61. The method of claim 60, wherein the administration is about 300 μg of the immunoconjugate dimer.

62. The method of claim 60, wherein the administration is about 600 μg of the immunoconjugate dimer.

63. The method of any one of claims 1-3 or 65-67, wherein the outcome is measured at least 6 months after beginning treatment.

64. The method of any one of claims 1-63, wherein the patient is a human.

65. A method for preventing, inhibiting, or reversing wet age-related macular degeneration (AMD) in an eye of a patient in need thereof, comprising, administering to the patient in multiple dosing sessions:
(a) an effective amount of an immunoconjugate comprising two dimerized immunoglobulin (Ig) Fc monomers and a mutated factor VII protein, wherein the mutated factor VII protein is fused to only one of the Fc monomers; and
(b) a VEGF inhibitor;
wherein the administration results in an improved best-corrected visual acuity (BCVA) outcome, improved reduction in mean choroidal neovascularization (CNV) lesion area, improved reduction in CNV exudation, or improved durability of treatment compared to patients having been administered the VEGF inhibitor alone.

66. A method for preventing, inhibiting, or reversing ocular neovascularization in an eye of a patient in need thereof, comprising, administering to the patient in multiple dosing sessions:
(a) an effective amount of an immunoconjugate comprising two dimerized immunoglobulin (Ig) Fc monomers and a mutated factor VII protein, wherein the mutated factor VII protein is fused to only one of the Fc monomers; and
(b) a VEGF inhibitor;
wherein the administration results in an improved best-corrected visual acuity (BCVA) outcome, improved reduction in mean choroidal neovascularization (CNV) lesion area, improved reduction in CNV exudation, or improved durability of treatment compared to patients having been administered the VEGF inhibitor alone.

67. A method for preventing, inhibiting, or reversing tumor neovascularization in an eye of a patient in need thereof, comprising, administering to the patient in multiple dosing sessions:
(c) an effective amount of an immunoconjugate comprising two dimerized immunoglobulin (Ig) Fc monomers and a mutated factor VII protein, wherein the mutated factor VII protein is fused to only one of the Fc monomers; and
(d) a VEGF inhibitor;
wherein the administration results in an improved best-corrected visual acuity (BCVA) outcome, improved reduction in mean choroidal neovascularization (CNV) lesion area, improved reduction in CNV exudation, or improved durability of treatment compared to patients having been administered the VEGF inhibitor alone.

68. The method of any one of claims 65-67, wherein the mutated factor VII protein exhibits a decreased coagulation response in a mammalian host, as compared to a wild-type factor VII protein.


SEQUENCE LISTING (5 sequences)

SEQ ID NO: 1 - DNA, 2072 bp, Artificial Sequence
  Human factor VII active site mutant immunoconjugate mRNA, complete cds
aagctttgcagagatttcatcatggtctcccaggccctcaggctcctctgccttctgcttgggcttcagggctgcctggctgcagtcttcgtaacccaggaggaagcccacggcgtcctgcaccggcgccggcgcgccaacgcgttcctggaggagctgcggccgggctccctggagagggagtgcaaggaggagcagtgctccttcgaggaggcccgggagatcttcaaggacgcggagaggacgaagctgttctggatttcttacagtgatggtgaccagtgtgcctcaagtccatgccagaatgggggctcctgcaaggaccagctccagtcctatatctgcttctgcctccctgccttcgagggccggaactgtgagacgcacaaggatgaccagctgatctgtgtgaacgagaacggcggctgtgagcagtactgcagtgaccacacgggcaccaagcgctcctgtcggtgccacgaggggtactctctgctggcagacggggtgtcctgcacacccacagttgaatatccatgtggaaaaatacctattctagaaaaaagaaatgccagcaagccccaagggcgaattgtggggggcaaggtgtgccccaaaggggagtgtccatggcaggtcctgttgttggtgaatggagctcagttgtgtggggggaccctgatcaacaccatctgggtggtctccgcggcccactgtttcgacaaaatcaagaactggaggaacctgatcgcggtgctcggggagcacgacctcagcgagcacgacggggatgagcagagccggcgggtggcgcaggtcatcatccccagcacgtacgtcccgggcaccaccaaccacgacatcgcgctgctccgcctgcaccagcccgtggtcctcactgaccatgtggtgcccctctgcctgcccgaacggacgttctctgagaggacgctggccttcgtgcgcttctcattggtcagcggctggggccagctgctggaccgtggcgccacggccctggagctcatggtcctcaacgtgccccggctgatgacccaggactgcctgcagcagtcacggaaggtgggagactccccaaatatcacggagtacatgttctgtgccggctactcggatggcagcaaggactcctgcgcgggggacagtggaggcccacatgccacccactaccggggcacgtggtacctgacgggcatcgtcagctggggccagggctgcgcaaccgtgggccactttggggtgtacaccagggtctcccagtacatcgagtggctgcaaaagctcatgcgctcagagccacgcccaggagtcctcctgcgagccccatttcccggatccgcagagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgataagcggccgc

SEQ ID NO: 2 - protein, 641 aa, Artificial Sequence
  Human factor VII mutant immunoconjugate AA sequence
  MOD_RES (6)..(6): May be glutamic acid or a post translationally modified gamma carboxy-glutamic acid (GLA)
  MOD_RES (7)..(7): May be glutamic acid or a post translationally modified gamma carboxy-glutamic acid (GLA)
  MOD_RES (14)..(14): May be glutamic acid or a post translationally modified gamma carboxy-glutamic acid (GLA)
  MOD_RES (16)..(16): May be glutamic acid or a post translationally modified gamma carboxy-glutamic acid (GLA)
  MOD_RES (19)..(19): May be glutamic acid or a post translationally modified gamma carboxy-glutamic acid (GLA)
  MOD_RES (20)..(20): May be glutamic acid or a post translationally modified gamma carboxy-glutamic acid (GLA)
  MOD_RES (25)..(25): May be glutamic acid or a post translationally modified gamma carboxy-glutamic acid (GLA)
  MOD_RES (26)..(26): May be glutamic acid or a post translationally modified gamma carboxy-glutamic acid (GLA)
  MOD_RES (29)..(29): May be glutamic acid or a post translationally modified gamma carboxy-glutamic acid (GLA)
  MOD_RES (34)..(34): May be glutamic acid or a post translationally modified gamma carboxy-glutamic acid (GLA)
  CARBOHYD (52)..(52): May be site of O-glycosylation
  CARBOHYD (60)..(60): May be site of O-glycosylation
  CARBOHYD (145)..(145): May be site of N-glycosylation
  CARBOHYD (322)..(322): May be site of N-glycosylation
  CARBOHYD (491)..(491): May be site of N-glycosylation
ANAFLEELRPGSLERECKEEQCSFEEAREIFKDAERTKLFWISYSDGDQCASSPCQNGGSCKDQLQSYICFCLPAFEGRNCETHKDDQLICVNENGGCEQYCSDHTGTKRSCRCHEGYSLLADGVSCTPTVEYPCGKIPILEKRNASKPQGRIVGGKVCPKGECPWQVLLLVNGAQLCGGTLINTIWVVSAAHCFDKIKNWRNLIAVLGEHDLSEHDGDEQSRRVAQVIIPSTYVPGTTNHDIALLRLHQPVVLTDHVVPLCLPERTFSERTLAFVRFSLVSGWGQLLDRGATALELMVLNVPRLMTQDCLQQSRKVGDSPNITEYMFCAGYSDGSKDSCAGDSGGPHATHYRGTWYLTGIVSWGQGCATVGHFGVYTRVSQYIEWLQKLMRSEPRPGVLLRAPFPGSAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 641 aa, Artificial Sequence
  Human factor VII mutant immunoconjugate AA sequence
  MOD_RES (6)..(6): May be glutamic acid or a post translationally modified gamma carboxy-glutamic acid (GLA)
  MOD_RES (7)..(7): May be glutamic acid or a post translationally modified gamma carboxy-glutamic acid (GLA)
  MOD_RES (14)..(14): May be glutamic acid or a post translationally modified gamma carboxy-glutamic acid (GLA)
  MOD_RES (16)..(16): May be glutamic acid or a post translationally modified gamma carboxy-glutamic acid (GLA)
  MOD_RES (19)..(19): May be glutamic acid or a post translationally modified gamma carboxy-glutamic acid (GLA)
  MOD_RES (20)..(20): May be glutamic acid or a post translationally modified gamma carboxy-glutamic acid (GLA)
  MOD_RES (25)..(25): May be glutamic acid or a post translationally modified gamma carboxy-glutamic acid (GLA)
  MOD_RES (26)..(26): May be glutamic acid or a post translationally modified gamma carboxy-glutamic acid (GLA)
  MOD_RES (29)..(29): May be glutamic acid or a post translationally modified gamma carboxy-glutamic acid (GLA)
  MOD_RES (34)..(34): May be glutamic acid or a post translationally modified gamma carboxy-glutamic acid (GLA)
  CARBOHYD (52)..(52): May be site of O-glycosylation
  CARBOHYD (60)..(60): May be site of O-glycosylation
  CARBOHYD (145)..(145): May be site of N-glycosylation
  CARBOHYD (322)..(322): May be site of N-glycosylation
  CARBOHYD (491)..(491): May be site of N-glycosylation
ANAFLEELRPGSLERECKEEQCSFEEAREIFKDAERTKLFWISYSDGDQCASSPCQNGGSCKDQLQSYICFCLPAFEGRNCETHKDDQLICVNENGGCEQYCSDHTGTKRSCRCHEGYSLLADGVSCTPTVEYPCGKIPILEKRNASKPQGRIVGGKVCPKGECPWQVLLLVNGAQLCGGTLINTIWVVSAAHCFDKIKNWRNLIAVLGEHDLSEHDGDEQSRRVAQVIIPSTYVPGTTNHDIALLRLHQPVVLTDHVVPLCLPERTFSERTLAFVRFSLVSGWGQLLDRGATALELMVLNVPRLMTQDCLQQSRKVGDSPNITEYMFCAGYSDGSKDSCKGDAGGPHATHYRGTWYLTGIVSWGQGCATVGHFGVYTRVSQYIEWLQKLMRSEPRPGVLLRAPFPGSAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - DNA, 2057 bp, Artificial Sequence
  Coding sequence for human factor VII mutant immunoconjugate AA sequence
ggatccaccatggtgtcacaagccctccgcctgctctgcctccttctcggacttcaaggatgtctggccgcagtgttcgtgactcaagaagaggcccacggggtgctgcaccggaggcgccgcgcgaacgcctttctggaggagctgcggccaggctcgctggaaagagagtgcaaagaggagcaatgctccttcgaggaggccagagaaattttcaaggacgccgaacggactaagctgttttggatctcatacagcgacggcgaccagtgtgccagcagcccgtgccagaacggcggttcgtgcaaggaccagctgcagtcctacatttgtttctgcctgccggcgttcgaaggaagaaactgtgaaacccacaaggatgaccagctgatctgcgtgaacgagaacggaggctgcgaacagtactgctccgaccacacggggactaagaggagctgcagatgccatgagggctatagcctgctggccgacggggtgtcctgcactccgactgtggaatacccgtgcggaaagatccccatcctggagaagcggaacgcatccaagccccaaggacgaattgtcggcggaaaggtctgccctaagggagagtgtccttggcaagtgctcctcttggtgaacggagcgcagctttgcggcggaactctgatcaacaccatctgggtggtgtcggcagctcactgctttgataagattaagaactggcggaacctgatcgcggtgcttggcgaacatgacctgtccgaacacgacggggacgagcagtcccggagagtggcccaggtcatcattccatccacctacgtgccggggaccacaaaccacgacatcgccttgctgcggctgcaccagccggtggtccttaccgatcacgtcgtgccgctctgcctgcccgagcgcacctttagcgagcgcaccctggccttcgtgcgcttctccctggtgtccggctggggacagctgctggacagaggcgccaccgccctggaactgatggtgctgaacgtgccacggctcatgacccaagattgtctgcagcagtcgcgcaaagtcggggattcaccgaacatcaccgagtacatgttctgcgccggttactccgatggatcgaaggactcgtgcgctggagactccggtggaccccatgctacccattatcgcggtacttggtacctgactggtattgtgtcctggggacagggctgcgccaccgtgggacatttcggcgtgtacacaagggtgtcacagtacatcgaatggttgcagaagctcatgcgcagcgaacctcgcccgggagtgctcctgagggcccctttccccggaagcgctgagcctaagagctgcgacaagacccacacttgtccgccatgccccgcgcccgaactgctgggtggcccatccgtgttcctgttcccgcctaagcccaaggacaccctgatgatttcgcggacccccgaagtgacttgcgtggtggtggacgtcagccacgaagatccggaagtcaagttcaattggtacgtggatggcgtcgaagtccataacgccaagaccaagccccgcgaggagcagtacaattccacttaccgggtggtgtccgtgttgaccgtgttgcaccaggactggctgaacggaaaggaatacaagtgcaaagtgtcgaataaggccctcccggccccgatcgagaaaacgatctccaaggccaagggccagcctcgggagccccaggtctacaccttgccgccgtcccgggatgagctgaccaagaaccaggtgtcactcacctgtctcgtcaaggggttctacccttccgacatcgccgtggaatgggagtcgaacgggcagccggaaaacaattacaagaccactcctcctgtcctggattccgacgggtcattcttcctgtactcaaagctgaccgtggacaagagcagatggcaacagggcaacgtgttcagctgctccgtgatgcacgaggccctgcacaaccactacacccagaagtccctgtccctctctcccggaaaatgagcggccgc

SEQ ID NO: 5 - DNA, 2057 bp, Artificial Sequence
  Coding sequence for human factor VII mutant immunoconjugate AA sequence
tctagaccaccatggtgtcacaagccctccgcctgctctgcctccttctcggacttcaaggatgtctggccgcagtgttcgtgactcaagaagaggcccacggggtgctgcaccggaggcgccgcgcgaacgcctttctggaggagctgcggccaggctcgctggaaagagagtgcaaagaggagcaatgctccttcgaggaggccagagaaattttcaaggacgccgaacggactaagctgttttggatctcatacagcgacggcgaccagtgtgccagcagcccgtgccagaacggcggttcgtgcaaggaccagctgcagtcctacatttgtttctgcctgccggcgttcgaaggaagaaactgtgaaacccacaaggatgaccagctgatctgcgtgaacgagaacggaggctgcgaacagtactgctccgaccacacggggactaagaggagctgcagatgccatgagggctatagcctgctggccgacggggtgtcctgcactccgactgtggaatacccgtgcggaaagatccccatcctggagaagcggaacgcatccaagccccaaggacgaattgtcggcggaaaggtctgccctaagggagagtgtccttggcaagtgctcctcttggtgaacggagcgcagctttgcggcggaactctgatcaacaccatctgggtggtgtcggcagctcactgctttgataagattaagaactggcggaacctgatcgcggtgcttggcgaacatgacctgtccgaacacgacggggacgagcagtcccggagagtggcccaggtcatcattccatccacctacgtgccggggaccacaaaccacgacatcgccttgctgcggctgcaccagccggtggtccttaccgatcacgtcgtgccgctctgcctgcccgagcgcacctttagcgagcgcaccctggccttcgtgcgcttctccctggtgtccggctggggacagctgctggacagaggcgccaccgccctggaactgatggtgctgaacgtgccacggctcatgacccaagattgtctgcagcagtcgcgcaaagtcggggattcaccgaacatcaccgagtacatgttctgcgccggttactccgatggatcgaaggactcgtgcgctggagactccggtggaccccatgctacccattatcgcggtacttggtacctgactggtattgtgtcctggggacagggctgcgccaccgtgggacatttcggcgtgtacacaagggtgtcacagtacatcgaatggttgcagaagctcatgcgcagcgaacctcgcccgggagtgctcctgagggcccctttccccggaagcgctgagcctaagagctgcgacaagacccacacttgtccgccatgccccgcgcccgaactgctgggtggcccatccgtgttcctgttcccgcctaagcccaaggacaccctgatgatttcgcggacccccgaagtgacttgcgtggtggtggacgtcagccacgaagatccggaagtcaagttcaattggtacgtggatggcgtcgaagtccataacgccaagaccaagccccgcgaggagcagtacaattccacttaccgggtggtgtccgtgttgaccgtgttgcaccaggactggctgaacggaaaggaatacaagtgcaaagtgtcgaataaggccctcccggccccgatcgagaaaacgatctccaaggccaagggccagcctcgggagccccaggtctacaccttgccgccgtcccgggatgagctgaccaagaaccaggtgtcactcacctgtctcgtcaaggggttctacccttccgacatcgccgtggaatgggagtcgaacgggcagccggaaaacaattacaagaccactcctcctgtcctggattccgacgggtcattcttcctgtactcaaagctgaccgtggacaagagcagatggcaacagggcaacgtgttcagctgctccgtgatgcacgaggccctgcacaaccactacacccagaagtccctgtccctctctcccggaaaatgaaccggt